(19) United States
(12) Patent Application Publication
(10) Pub. No.: US 2025/0325721 A1
(43) Pub. Date: Oct. 23, 2025

(54) SYSTEM AND DEVICE FOR REDUCING MICROBIAL BURDEN ON A SURFACE

(71) Applicant: Sterifre Medical, Inc., Kirkland, WA (US)

(72) Inventors: Czeslaw Golkowski, Seattle, WA (US); Mark Golkowski, Ithaca, NY (US); Josh Yasbek, Lyle, WA (US); John Geile, Vancouver, WA (US); Tristan Dudik, Vancouver, WA (US); Robert Callaway, Vancouver, WA (US); Daniel Weeks, Portland, OR (US); Luis Elenes, Terrebonne, OR (US); Wataru Nishizawa, Maple Valley, WA (US)

(21) Appl. No.: 18/713,579

(22) PCT Filed: Nov. 23, 2022
(86) PCT No.: PCT/US2022/080404

§ 371 Date: May 24, 2024

Related U.S. Application Data
(60) Provisional application No. 63/283,084, filed on Nov. 24, 2021.

(51) Int. Cl. A61L 2/20; A61L 2/18; A61L 2/22; A61L 2/24; B01D 46/00; B01D 46/62

(52) U.S. Cl. CPC A61L 2/202; A61L 2/186; A61L 2/22; A61L 2/24; B01D 46/0095; B01D 46/62; A61L 2202/11; A61L 2202/121; A61L 2202/122; A61L 2202/123; A61L 2202/14; A61L 2202/15; A61L 2202/24

(57) ABSTRACT

A system for reducing the viability of microorganisms on a surface is provided herein. The system includes a housing with a front panel and a rear panel, an inlet filter and an exhaust filter, a chamber for receiving an item to be disinfected/sterilized, a cartridge engagement for receiving a disinfectant/sterilant cartridge, an ozone generation system comprising a duct and an ozone generator, an evaporator including an inlet, outlet and a reservoir, a nebulizer, the nebulizer fluidly connected to the reservoir of the evaporator, a ducting with a blower and four pathways for receiving and delivering airflow, a vent positioned on the rear panel of the housing, wherein airflow exits the housing through the vent, and an ambient ozone sensor to detect a presence of ozone at a preset threshold value within the airflow, wherein airflow exiting the housing through the vent is to first flow past the ambient ozone sensor.

### BACKGROUND

### Technical Field

Several embodiments of the present disclosure relate generally to the art of generating atmospheres having sterilizing, disinfecting, sanitizing, decontaminating, and/or therapeutic aspects, and more particularly to sterilization, disinfection, sanitization, and/or decontamination of therapeutic devices, as well as related systems and methods.

### Description of the Related Art

Sterilization, disinfection, sanitization, and decontamination methods are used in a broad range of applications. A variety of methods is used, including steam, chemicals, fumigants, radiation, among others. Drawbacks to these methods exist and are addressed by the systems and methods disclosed herein.

### SUMMARY

As disclosed herein, a variety of items or surfaces may require processing in order to reduce the bioburden and decrease risk of infections. For example, critical items (such as surgical instruments, which contact sterile tissue), semi-critical items (such as endoscopes, which contact mucous membranes), and noncritical items (such as stethoscopes, which contact only intact skin) require various types of treatment, for example sterilization, high-level disinfection, and low-level disinfection, respectively. The present disclosure provides for various systems and methods for disinfecting/sanitizing various items (e.g., medical devices or electronics) and surfaces (e.g., workspaces, patient rooms, organic material, including but not limited to patient wounds).

Various systems and methods are provided for herein in order to accomplish disinfection of one or more items, surfaces etc. Additionally, in several embodiments the systems and methods are configured to allow low- or high-level disinfection. In still additional embodiments, the systems and methods are configured to allow sterilization.

For example, provided for herein in several embodiments is a system for reducing the viability of microorganisms on a surface. In some embodiments, the disclosed system includes a housing including a front panel and a rear panel. In some embodiments, the system includes a filter holder positioned within the front panel, the filter holder including a first opening and a second opening for receiving an inlet filter and an exhaust filter. In some embodiments, the system includes a chamber configured to receive an item to be disinfected, sterilized, or sanitized. In some embodiments, the system includes a cartridge engagement mechanism configured to receive a removable cartridge containing a volume of disinfectant/sterilant. In some embodiments, the system includes an ozone generation system with a duct comprising an inlet and an outlet and an ozone generator positioned along the length of the duct. In some embodiments, the system includes an evaporator with an inlet, an outlet, and a reservoir. In some embodiments, the system includes a nebulizer including an inlet, an outlet, a chamber, a cap. In some embodiments, the nebulizer can convert hydrogen peroxide into a vapor, and is fluidly connected to the reservoir of the evaporator. In some embodiments, the system includes a ducting including a blower, a first pathway configured to receive an airflow from the blower, a second pathway configured to deliver the airflow to the exhaust filter, a third pathway configured to receive the airflow from the inlet filter, and a fourth pathway configured to deliver the airflow to the ozone generation system. In some embodiments, the system includes a vent positioned on the rear panel of the housing, wherein airflow exits the housing through the vent. In some embodiments, the system includes an ambient ozone sensor configured to detect a presence of ozone at a preset threshold value within the airflow, wherein airflow exiting the housing through the vent is configured to first flow past the ambient ozone sensor.

In other embodiments, the system is configured to stop operating if the ambient ozone sensor detects the presence of ozone above the preset threshold value.

In other embodiments, the chamber comprises a base comprising a plurality of inlet openings on a first side of the base and a plurality of outlet openings on a second side of the base. In other embodiments, the plurality of inlet openings in the chamber is configured to receive airflow from the outlet of the evaporator. In other embodiments, the plurality of outlet openings in the chamber is configured to direct airflow to the blower of the ducting. In other embodiments, the chamber includes an inlet panel on a first side of the chamber and an outlet panel on a second side of the chamber. In other embodiments, the inlet panel, and the outlet panel of the chamber each comprise a plurality of openings to allow airflow through the inlet panel and the outlet panel. In other embodiments, the inlet panel, and the outlet panel of the chamber each comprise a plurality of hooks, wherein at least one of the plurality of hooks on the inlet panel and at least one of the corresponding plurality of hooks on the outlet panel are configured to retain at least one wire rack. In other embodiments, at least one wire rack is removable and repositionable by a user. In other embodiments, the outlet panel further includes a filter positioned behind the outlet panel and a filter support to retain the filter to the outlet panel.

In other embodiments, the front panel includes a first door configured to seal the chamber. In other embodiments, the front panel includes a second door configured to allow the user access to the cartridge containing the disinfectant or sterilant. In other embodiments, the front panel includes a third door configured to allow the user access to the filter holder containing the inlet filter and the exhaust filter.

In other embodiments, the inlet filter comprises an activated carbon filter or a high efficiency particulate air (HEPA) filter or both. In other embodiments, the outlet filter comprises an activated carbon filter or a high efficiency particulate air (HEPA) filter or both. In other embodiments, the airflow moves out of the outlet filter and into the housing. In other embodiments, the inlet filter includes an actuator that can be actuated if the inlet filter is not properly positioned or malfunctioning, wherein the exhaust filter comprises an actuator that can be actuated if the outlet filter is not properly positioned of malfunctioning, and wherein the system generates an error message to indicate to a user that at least one of the inlet filter and the exhaust filter is malfunctioning or improperly placed.

In other embodiments, the ozone generator further includes a pair of bars with a pair of electrode centers extending through a pair of glass tubes, wherein each of the pair of electrode centers comprise aluminum. In other embodiments, the pair of bars extend parallel to the length of the duct of the ozone generation system. In other embodiments, the ozone generation system further includes a power supply, wherein the power supply is configured to provide a constant supply of voltage to the ozone generator. In other embodiments, the system generates an error message when the power supply is unable to provide a constant supply of voltage to the ozone generator. In other embodiments, the airflow is moved parallel to the pair of bars of the ozone generator. In other embodiments, the duct of the ozone generation system includes a plurality of fins that are configured to serve as guide vanes to direct airflow and to reduce pressure drops along a length of the duct of the ozone generation system.

In other embodiments, the ozone generation system further includes an ozone sensor wherein the ozone sensor is configured to detect the amount of ozone in the airflow through the ozone generation system and can adjust a duty cycle of the ozone generator to keep the concentration of ozone in the airflow within a preset range. In other embodiments, the ozone sensor can determine the density of air and adjust a duty cycle of the ozone generator.

In other embodiments the reservoir includes a collection point that forms the lowest point of the reservoir. In other embodiments, the collection point can catch excess disinfectant/sterilant. In other embodiments, the excess disinfectant/sterilant of the collection point is configured to flow into the nebulizer. In other embodiments, the reservoir includes an outlet fluidly connected to the inlet of the nebulizer and an inlet fluidly connected to the outlet of the nebulizer.

In other embodiments, the nebulizer includes a predetermined level of disinfectant/sterilant in the chamber. In other embodiments, the system includes a reservoir pump configured to pump disinfectant/sterilant from the collection point of the reservoir to the chamber of the nebulizer. In other embodiments, the system includes a cartridge pump configured to pump disinfectant/sterilant from the cartridge to the chamber. In other embodiments, the cap includes a curved surface that allows excess disinfectant/sterilant to flow back to the chamber of the nebulizer. In other embodiments, the nebulizer includes a pizeocrystal configured to vibrate at a predetermined range to generate a mist of disinfectant/sterilant. In other embodiments, airflow is configured to flow through the inlet of the of the nebulizer, past a portion of the chamber of the nebulizer, and out of the outlet of the nebulizer.

In other embodiments, a first portion of airflow flows into the reservoir of the evaporator and a second portion of airflow flows into the nebulizer. In other embodiments, the first portion is greater than the second portion. In other embodiments, the first portion comprises between 95% to 99.5% of the airflow flowing into the evaporator, and wherein the second portion comprises between 0.5% to 5.0% of the airflow flowing into the evaporator.

In other embodiments, the evaporator includes a central hub, a first pathway, a second pathway, a third pathway, and a valve. In other embodiments, the first pathway can deliver airflow from the inlet to the central hub. In other embodiments, the second pathway can deliver airflow from the central hub, into the reservoir, and out of the outlet. In other embodiments, the third pathway can deliver airflow from the central hub to the outlet. In other embodiments, the valve can be retained within the central hub, wherein the valve is configured to move between a first position and a second position. In other embodiments, the valve rotates 60 degrees between the first position and the second position. In other embodiments, the first position of the valve can allow airflow from the inlet to the reservoir, and out of the outlet. In other embodiments, the second position of the valve is configured to allow airflow from the inlet to the outlet and bypasses the reservoir. In other embodiments, the outlet can direct airflow into the chamber. In other embodiments, the evaporator includes a pad for capturing droplets of disinfectant/sterilant.

In other embodiments, the ducting can include a valve configured to move between a first position and a second position. In other embodiments, the valve can rotate 90 degrees between the first position and the second position. In other embodiments, the first position of the valve can allow airflow from the blower to the ozone generation system. In other embodiments, the second position of the valve can allow airflow from the blower to the exhaust filter and allow airflow from the intake filter In other embodiments, the ducting includes a muffler positioned in the first pathway adjacent to the blower. In other embodiments, the muffler includes muffler foam. In other embodiments, the ducting includes a plurality of fins that can serve as guide vanes to direct airflow and to reduce pressure drops along a length of the duct of the ozone generation system.

In some embodiments, disclosed is a system for reducing the viability of microorganisms on a surface. In some embodiments, the system includes a housing including a front panel and a rear panel. In some embodiments, the system includes an inlet filter and an exhaust filter. In some embodiments, the system includes a chamber includes a base, an inlet panel on a first side of the chamber, an outlet panel on a second side of the chamber, a plurality of inlet openings on a first side of the base, and a plurality of outlet openings on a second side of the base configured to receive an item to be disinfected, sterilized, or sterilized. In some embodiments, the system includes a cartridge containing a volume of disinfectant/sterilant. In some embodiments, the system includes an ozone generation system. In some embodiments, the system includes an evaporator comprising an inlet, an outlet, and a reservoir. In some embodiments, the system includes a nebulizer configured to convert hydrogen peroxide into a vapor, and wherein the nebulizer is fluidly connected to the reservoir of the evaporator. In some embodiments, the system includes a vent positioned on the rear panel of the housing, wherein airflow exits the housing through the vent. In some embodiments, the system includes an ambient ozone sensor configured to detect a presence of ozone at a preset threshold value within the airflow, wherein airflow exiting the housing through the vent is configured to first flow past the ambient ozone sensor.

In other embodiments, the plurality of inlet openings of the chamber can receive airflow from the outlet of the evaporator. In other embodiments, the plurality of outlet openings of the chamber can direct airflow to a blower. In other embodiments, the inlet panel and the outlet panel of the chamber each include a plurality of openings to allow airflow through the inlet panel and the outlet panel. In other embodiments, the inlet panel and the outlet panel of the chamber each comprise a plurality of hooks, wherein at least one of the plurality of hooks on the inlet panel and at least one of the corresponding plurality of hooks on the outlet panel are configured to retain at least one wire rack. In other embodiments, the at least one wire rack of the chamber is removable and repositionable by a user. In other embodiments, the outlet panel of the chamber further comprises a filter positioned behind the outlet panel and a filter support configured to retain the filter to the outlet panel.

In some embodiments, disclosed is a system for reducing the viability of microorganisms on a surface. In some embodiments, the system includes a housing comprising a front panel and a rear panel. In some embodiments, the system includes an inlet filter and an exhaust filter. In some embodiments, the system includes a chamber configured to receive an item to be disinfected, sterilized, or sanitized. In some embodiments, the system includes a cartridge containing a volume of disinfectant/sterilant. In some embodiments, the system includes an ozone generation system including a duct comprising an inlet and an outlet. In some embodiments, the ozone generation system includes an ozone generator positioned along the length of the duct. In some embodiments, the ozone generator includes a pair of bars including a pair of electrode centers extending through a pair of glass tubes, wherein each of the pair of electrode centers comprise aluminum. In some embodiments, the ozone generator includes a power supply configured to provide a constant supply of voltage to the ozone generator. In some embodiments, the system includes an evaporator comprising an inlet, an outlet, and a reservoir. In some embodiments, the system includes a nebulizer configured to convert hydrogen peroxide into a vapor, and wherein the nebulizer is fluidly connected to the reservoir of the evaporator. In some embodiments, the system includes a blower. In some embodiments, the system includes a vent positioned on the rear panel of the housing, wherein airflow exits the housing through the vent. In some embodiments the system includes an ambient ozone sensor configured to detect a presence of ozone at a preset threshold value within the airflow, wherein airflow exiting the housing through the vent is configured to first flow past the ambient ozone sensor.

In other embodiments, the pair of bars of the ozone generator extend parallel to the length of the duct of the ozone generation system. In other embodiments, the system is configured to generate an error message when the power supply of the ozone generator is unable to provide a constant supply of voltage to the ozone generator. In other embodiments, the airflow through the ozone generator is moved parallel to the pair of bars of the ozone generator. In other embodiments, the duct of the ozone generation system includes a plurality of fins that are configured to serve as guide vanes to direct airflow and to reduce pressure drops along a length of the duct of the ozone generation system. In other embodiments, the ozone generation system includes an ozone sensor configured to detect the amount of ozone in the airflow through the ozone generation system and is configured to adjust a duty cycle of the ozone generator to keep the concentration of ozone in the airflow within a preset range. In other embodiments, the ozone sensor is configured to determine the density of air and to adjust a duty cycle of the ozone generator.

In some embodiments, disclosed is a system for reducing the viability of microorganisms on a surface. In some embodiments, the system includes a housing comprising a front panel and a rear panel. In some embodiments, the system includes an inlet filter and an exhaust filter. In some embodiments, the system includes a chamber is configured to receive an item to be disinfected, sterilized, or sanitized. In some embodiments, the system includes a cartridge containing a volume of disinfectant/sterilant. In some embodiments, the system includes an ozone generation system. In some embodiments, the system includes an evaporator comprising an inlet and an outlet. In some embodiments, the evaporate includes a reservoir comprising a collection point forming the lowest point of the reservoir and configured to retain excess disinfectant/sterilant. In some embodiments, the ozone generation system of the system includes a ducting including a central hub, a first pathway configured to deliver airflow from the inlet to the central hub, a second pathway configured to deliver airflow from the central hub, into the reservoir, and out of the outlet, a third pathway configured to deliver airflow from the central hub to the outlet. In some embodiments, the ozone generator includes a valve positioned within the central hub, wherein the valve is configured to move between a first position and a second position. In some embodiments the system includes a nebulizer configured to convert hydrogen peroxide into a vapor, and wherein the nebulizer is fluidly connected to the reservoir of the evaporator. In some embodiments, the system includes a blower. In some embodiments, the system includes a vent positioned on the rear panel of the housing, wherein airflow exits the housing through the vent. In some embodiments, the system includes an ambient ozone sensor to detect a presence of ozone at a preset threshold value within the airflow, wherein airflow exiting the housing through the vent is configured to first flow past the ambient ozone sensor.

In other embodiments, the excess disinfectant/sterilant of the collection point of the reservoir is configured to flow into the nebulizer. In other embodiments, the reservoir of the evaporator comprises an outlet fluidly connected to an inlet of the nebulizer and an inlet fluidly connected to an outlet of the nebulizer. In other embodiments, the evaporator further comprises a pad for capturing droplets of disinfectant/sterilant. In other embodiments, of the air flowing into the evaporator, a first portion of airflow entering the evaporator flows into the reservoir of the evaporator and a second portion of airflow entering the evaporator flows into the nebulizer. In some embodiments, the first portion is greater than the second portion. In some embodiments, the first portion comprises between 95% to 99.5% of the airflow flowing into the evaporator, and wherein the second portion comprises between 0.5% to 5.0% of the airflow flowing into the evaporator. In other embodiments, the valve of the evaporator rotates 60 degrees between the first position and the second position. In some embodiments, the first position of the valve of the evaporator is configured to allow airflow from the inlet to the reservoir, and out of the outlet. In some embodiments, the second position of the valve of the evaporator is configured to allow airflow from the inlet to the outlet and bypasses the reservoir. In other embodiments, the outlet is configured to direct airflow into the chamber.

In some embodiments, disclosed is a system for reducing the viability of microorganisms on a surface. In some embodiments, disclosed is a housing comprising a front panel and a rear panel. In some embodiments, the system includes a housing comprising a front panel and a rear panel. In some embodiments, the systems include amber configured to receive an item to be disinfected, sterilized, or sanitized. In some embodiments, the system includes. a cartridge containing a volume of disinfectant/sterilant. In some embodiments, the system includes an ozone generation system. In some embodiments, disclosed is an evaporator comprising an inlet, an outlet, and a reservoir. In some embodiments, the system includes a nebulizer including an inlet, determined level of disinfectant/sterilant in the chamber. In some embodiments, disclosed is a cap, a piezocrystal configured to vibrate at a predetermined range to generate a mist of disinfectant/sterilant. In some embodiments, the nebulizer of the system is fluidly connected to the reservoir of the evaporator. In some embodiments, the nebulizer of the system is fluidly connected to the reservoir of the evaporator. In some embodiments, the system includes a blower. In some embodiments, the system includes a vent positioned on the rear panel of the housing, wherein airflow exits the housing through the vent. In some embodiments, the system includes an ambient ozone sensor configured to detect a presence of ozone at a preset threshold value within the airflow, wherein airflow exiting the housing through the vent first flows past the ambient ozone sensor.

In other embodiments, the nebulizer is fluidly connected to a reservoir pump configured to pump disinfectant/sterilant from the collection point of the reservoir to the chamber of the nebulizer. In other embodiments, the nebulizer is fluidly connected to a cartridge pump configured to pump disinfectant/sterilant from the cartridge to the chamber. In other embodiments, the cap of the nebulizer includes a curved surface that allows excess disinfectant/sterilant to flow back to the chamber of the nebulizer.

In other embodiments, airflow through the nebulizer flows through the inlet of the of the nebulizer, past a portion of the chamber of the nebulizer, and out of the outlet of the nebulizer. In some embodiments, a first portion of airflow flowing through the evaporator flows into the reservoir of the evaporator and a second portion of airflow flows into the nebulizer. In some embodiments, the first portion is greater than the second portion. In some embodiments, the first portion comprises between 95% to 99.5% of the airflow flowing into the evaporator, and wherein the second portion comprises between 0.5% to 5.0% of the airflow flowing into the evaporator.

In some embodiments, a method for reducing viable microbial burden on a surface is disclosed, the method comprising placing at least one item into a chamber of a system for reducing microorganism viability, wherein the system comprises a nebulizer configured to convert hydrogen peroxide solution into a vapor, a cartridge configured to contain the hydrogen peroxide solution, at least one peristaltic pump, an ozone generator, a blower, an inlet and an outlet. In some embodiments, the method includes activating a conditioning phase to circulate ozone from the ozone generator in the system, wherein the ozone is configured to convert H₂O molecules to OH radicals so as to reduce residual moisture in the system. In some embodiments, the method includes activating a disinfection phase wherein the hydrogen peroxide solution is nebulized into a spray and is circulated through the system. In some embodiments, the method includes activating a post-disinfection conditioning phase to circulate ozone from the ozone generator in the system, wherein the ozone is configured to neutralize any remaining H₂O₂ in the system. In some embodiments, the method includes activating a system clearing phase to pull air into the system through the inlet, circulate the air through the nebulizer and the chamber, and exhaust the air out of the outlet.

In some embodiments, the method includes a disinfection phase that operates at an ambient temperature between about 20° C. to 25° C. In some embodiments, the system of the disclosed method operates with an ambient relative humidity between about 20% and 60%. In some embodiments, the method includes conditioning phase that with a duration of at least 2.5 minutes. In some embodiments, the method includes a disinfection phase with a duration of at least 4.5 minutes. In some embodiments, the method includes a post-disinfection phase with a duration of at least 2 minutes. In some embodiments, the method includes a system clearing phase with a duration of at least 1 minute. In some embodiments, the system of the method does not include a heater configured to dry the system. In some embodiments, the system of the disclosed method does not include a humidifier or a dehumidifier. In some embodiments, the system of the disclosed method does not include a desiccator. In some embodiments, the fluid flow during the conditioning phase of the disclosed method circulates fluid flow that bypasses the nebulizer. In some embodiments, fluid flow during the disinfection phase of the disclosed method circulates fluid flow through the nebulizer. In some embodiments, fluid flow during the post-disinfection conditioning phase of the disclosed method circulates fluid flow through the nebulizer. In some embodiments, the fluid flow during the clearing phase of the disclosed method circulates fluid flow that bypasses the nebulizer.

In some embodiments, disclosed is a method for reducing viable microbial burden on a surface. In some embodiments the method includes placing at least one item into a chamber configured to contain the at least one item. In some embodiments, the method includes activating a conditioning phase. In some embodiments, the conditioning phase can include activating a fan to circulate air in a closed loop to circulate the chamber, activating an ozone generator to generate ozone, activating the fan to circulate air, including the ozone, in the closed loop between the ozone generator and the chamber. In some embodiments, the method can include activating a disinfection phase. In some embodiments, the disinfection phase can include pumping disinfectant with a peristaltic pump from a reservoir to a nebulizer, converting disinfectant into a vapor with the nebulizer, activating the fan to circulate air, including the vapor, in the closed loop between the nebulizer and the chamber, and activating the fan to circulate air, including the ozone, in the closed loop between the ozone generator and the chamber. In some embodiments, the method includes activating a post-disinfection conditioning phase. In some embodiments, the post-disinfection conditioning phase includes activating an ozone generator to generate ozone and activating the fan to circulate air, including the ozone, in the closed loop between the ozone generator, the nebulizer, and the chamber. In some embodiments, the method includes activating a system clearing phase. In some embodiments, the system clearing phase includes activating a valve to allow air to flow into the system through an inlet, activating a valve to allow air to flow out of the system through an outlet, and activating the fan to introduce the air through the inlet, into the chamber, and exhaust through the outlet.

In some embodiments, the method includes a temperature conditioning step. In some embodiments, the temperature conditioning step occurs during the purge phase. In some embodiments, the temperature conditioning step occurs during a second or any subsequent purge phase. In some embodiments, the temperature conditioning step can include activating a heating element to increase the temperature of the walls. In some embodiments, the heating element can comprise ductile heating wires. In some embodiments, the heating element can heat a carrier, like air, and a fan to circulate the air throughout the system. In some embodiments, the temperature conditioning increases the temperature of the walls sufficient to remove, for example, hydrogen peroxide from the inner chamber walls and other parts of the system.

In some embodiments, the method is performed in about 10 minutes. In some embodiments, the conditioning phase of the method is about 150 seconds in duration. In some embodiments, the post-disinfection conditioning phase of the method is about 2 minutes in duration. In some embodiments, the sterilization or disinfection phase of the method is about 4 minutes and 30 seconds to about 5 minutes in duration. In some embodiments, the system clearing phase of the method is about 60 seconds. In some embodiments, the system of the method includes an inlet that comprises a HEPA filter. In some embodiments the system clearing phase of the method further comprises closing a valve to allow the fan to push air through the outlet. In some embodiments, the system of the method includes an outlet that comprises an activated carbon filter and a high efficiency particulate air (HEPA) filter. In some embodiments, the method includes disinfectant at a concentration of between about 30% to 60%. In some embodiments, the method includes disinfectant at a concentration of about 50%. In some embodiments, the method includes disinfectant that is hydrogen peroxide. In some embodiments, the method includes hydrogen peroxide at a concentration of about 50%. In some embodiments, the method includes a reservoir with a replaceable cartridge. In some embodiments, the method is operated at a temperature between about 20° C. to 25° C. In some embodiments, the method is operated at a relative humidity between about 20% and 60%. In some embodiments, the method is operated at or below an ambient pressure. In some embodiments, the elevated temperature during the purge phase is performed in about 60 seconds.

In some embodiments, disclosed is an automated method for sterilizing or disinfecting at least one item. In some embodiments, the method includes receiving at least one item to be sterilized or disinfected into an interior volume of a chamber for sterilization or disinfection. In some embodiments, the chamber for sterilization or disinfection is part of a system comprising an inlet, an outlet port, an ozone generator, a sterilant generator, and a plurality of conduits configured to fluidly connect each of the inlet, sterilant generator, ozone generator, and the chamber. In some embodiments, the system includes at least one fan, configured to provide gaseous flow through the system. In some embodiments, the system includes a controller and a plurality of valves in respective conduits. In some embodiments, the method includes activating a conditioning phase by the controller, wherein the conditioning phase is configured to dry a surface of the at least one item in the chamber and internal flow conduits, wherein the controller activates the fan to move air, and wherein the valves are positioned by the controller to provide closed loop flow of air moved by the fan. In some embodiments, the method includes activating an disinfection phase by the controller, wherein the exposure phase is configured to disinfect the at least one item, wherein the controller causes the disinfectant generator to begin generating disinfectant, wherein the disinfectant comprises a mist of hydrogen peroxide generated from a solution of hydrogen peroxide in the disinfectant generator at a concentration of about 50%, wherein the valves are positioned by the controller to provide closed loop flow through the nebulizer so that disinfectant is delivered to the chamber for a pre-determined time to disinfect the at least one item. In some embodiments, the method includes activating a post-disinfection conditioning phase by the controller, wherein the post-disinfection phase introduces ozone generated by the ozone generator into the chamber containing residual hydrogen peroxide disinfectant to neutralize the disinfectant. In some embodiments, the method includes activating a system clearing phase by the controller, wherein the purge phase includes positioning the valves by the controller to allow open flow and to allow air to be pulled in through the inlet and force the gaseous water vapor and oxygen from the chamber and out the outlet, wherein each of the inlet and outlet comprise a respective filter.

In some embodiments, the automated method includes a controller that activates the fan to move air through the ozone generator to produce ozone. In some embodiments, the automated method includes a disinfectant comprising a vapor of hydrogen peroxide. In some embodiments, the automated method operates at a pre-programmed relative ambient humidity between about 20% to 60%. In some embodiments, the conditioning phase of the automated method is activated for about 180 seconds. In some embodiments, the disinfection phase of the automated method is activated for about 4 minutes and 30 seconds. In some embodiments, the post-disinfection conditioning phase of the automated method is activated for about 120 seconds. In some embodiments, the system clearing phase of the automated method is activated for about 60 seconds. In some embodiments, the system of the automated method is configured to receive a cartridge. In some embodiments, the automated method operates between an ambient temperature between about 20° C. to 25° C. In some embodiments, the sterilant of the automated method is delivered by a peristaltic pump. In some embodiments, in the automated method, at least one of the filters of the inlet and outlet is a HEPA filter. In some embodiments, in the automated method, at least one of the filters of the inlet and outlet is a charcoal filter.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1A illustrates a perspective view of an embodiment of a device for disinfection, sterilization, and/or sanitization.

FIG. 1B illustrates a rear view of the device of FIG. 1

FIG. 2A illustrates a front view of the device of FIG. 1 with a front panel of the housing removed.

FIG. 2B illustrates a front view of the device of FIG. 1 with the front panel of the housing, the filters, and cartridge removed.

FIGS. 3A-3C illustrate a plurality of views of a chamber of the device of FIG. 1.

FIG. 4A illustrates a perspective view of an inlet plate for the chamber of FIGS. 3A-3C.

FIG. 4B illustrates an exploded view of an outlet plate for the chamber of FIGS. 3A-3C.

FIG. 5A illustrates a front view of an embodiment of a filter holder positioned in the device of FIG. 1.

FIGS. 5B-5C illustrates a front and perspective view of the filter holder.

FIGS. 6A-6C illustrate a plurality of views of an embodiment of a filter for the device of FIG. 1.

FIGS. 7A-7C illustrate a plurality of views of an embodiment of a cartridge engagement mechanism.

FIGS. 7D-7F illustrate a plurality of views of an embodiment of an upper portion of the cartridge engagement mechanism illustrated in FIGS. 7A-7C.

FIG. 7G illustrates a front view of a cartridge in the cartridge engagement mechanism of FIGS. 7A-7C, wherein the handle of the cartridge engagement mechanism is in a first position.

FIG. 7H illustrates a front view of a cartridge in the cartridge engagement mechanism of FIGS. 7A-7C, wherein the handle of the cartridge engagement mechanism is in a second position.

FIG. 8A illustrates a perspective view of an embodiment of the cartridge.

FIGS. 8B-8C illustrate side and front views of the embodiment of the cartridge of FIG. 8A.

FIG. 8D illustrates a cross-sectional view of the closure of the cartridge of FIG. 8A.

FIG. 8E illustrates a cross-sectional view of the cartridge of FIG. 8A.

FIG. 8F illustrates a cross-sectional view of the closure of FIG. 8F, wherein the spout is in a first open position.

FIG. 8G illustrates a cross-sectional view of the closure of FIG. 8G, wherein the spout is in a second closed position.

FIGS. 9A-9B illustrate a perspective and side view of an embodiment of a closure of the cartridge of FIG. 8A.

FIGS. 10A-10D illustrate a plurality of views of another embodiment of a closure of the cartridge of FIG. 8A.

FIG. 11A illustrates a perspective view of the device of FIG. 1 with a rear panel of the housing removed.

FIGS. 11B-11C illustrate two side perspective views of the device of FIG. 1 with the rear panel of the housing removed.

FIG. 11D illustrates a perspective view of an embodiment of a main driver PCB with an attached leak rate sensor.

FIG. 12 illustrates a schematic view of fluid flow through the housing of the device of FIG. 1 after the fluid flow exits the components of FIGS. 14A-14B associated with fluid flow.

FIGS. 13A-13B illustrate a front and rear view of the components of the device of FIG. 1 associated with fluid flow.

FIG. 13C illustrates a front perspective view of the components of FIGS. 14A-14B associated with fluid flow.

FIG. 14 illustrates a perspective view of the components of FIGS. 14A-14C with the chamber removed.

FIGS. 15A-15B illustrates a rear and front view of an embodiment of an ozone generation system.

FIG. 15C illustrates a rear view of the ozone generation system of FIGS. 15A-15B with the ozone duct cap removed to show the ozone generator.

FIGS. 15D-15E illustrate a rear view and perspective view of an embodiment of the ozone duct 710 of the ozone generation system of FIGS. 15A-15C with the ozone duct cap removed.

FIGS. 16A-16B illustrate rear and perspective views of the ozone generator and the associated power supply of the ozone generation system of FIGS. 15A-15B.

FIGS. 16C-16E illustrate front, perspective, and cross-sectional views of the ozone generator of the ozone generation system of FIGS. 15A-15B.

FIG. 16F illustrates an exploded view of the ozone generator of the ozone generation system of FIGS. 15A-15B.

FIG. 17 illustrates a fluid flow through a rear view of the ozone generation system of FIGS. 15A-15B.

FIG. 18A illustrates a perspective view of an embodiment of ducting in the device of FIG. 1.

FIGS. 18B-18D illustrate a rear, bottom, and top view of the ducting in the device of FIG. 1 with the blower adaptor removed.

FIGS. 18E-18F illustrate a top view of the ducting of FIGS. 19A-19C with the bottom portion of the ducting removed.

FIG. 19A illustrates an embodiment of the ducting in FIG. 19E wherein a valve of the ducting is in a first position.

FIG. 19B illustrates an embodiment of the ducting in FIG. 19E wherein a valve of the ducting is in a second position.

FIG. 20A illustrates an embodiment of an evaporator and a fluidly connected nebulizer.

FIG. 20B illustrates a cross-sectional view of the evaporator and fluidly connected nebulizer of FIG. 21A.

FIGS. 21A-21B illustrates a front and rear view of the evaporator of FIG. 21A.

FIGS. 21C-21D illustrates a top and bottom view of the evaporator of FIG. 21A.

FIGS. 21E-21F illustrate a front and rear perspective views of the evaporator of FIG. 21A.

FIG. 21G illustrates a top view of the evaporator of FIG. 21A with the cap removed.

FIG. 22A illustrates an embodiment of a top view of the evaporator of FIG. 21 wherein a valve of the evaporator is in a first position.

FIG. 22B illustrates an embodiment of a top view of the evaporator of FIG. 21 wherein the valve of the evaporator is in a second position.

FIGS. 23A-23C illustrate perspective and cross-sectional views of the nebulizer of FIG. 21.

FIG. 23D illustrates a schematic view of fluid flow through a cross-sectional view of the nebulizer of FIG. 21A.

FIGS. 24A-24B illustrates a schematic of cavitation behavior within the nebulizer of FIGS. 23A-23B.

FIGS. 24C-24D illustrate various cone heights of the nebulizer of FIGS. 24C-24D.

FIG. 25 illustrates an enlarged view of a level sensor on the nebulizer of FIGS. 23A-23C.

FIG. 26A illustrates a schematic of an embodiment of a system for reducing microorganisms on a surface.

FIG. 26B illustrates a flow chart of an embodiment of a method for reducing microorganisms on a surface.

FIG. 26C illustrates an embodiment of the schematic of FIG. 26A during a first Phase of the flow chart of FIG. 10, wherein a chamber of the system for reducing microorganisms on a surface is being conditioned.

FIG. 26D illustrates an embodiment of the schematic of FIG. 26A during a second Phase of the flow chart of FIG. 26B, wherein an item placed in the chamber of the system for reducing microorganisms on a surface is being disinfect, sterilized, or sanitized.

FIG. 26E illustrates an embodiment of the schematic of FIG. 26A during a third Phase of the flow chart of FIG. 26B, wherein the chamber of the system for sterilization, disinfection, and sanitization is conditioned after the item placed in the chamber has been disinfected, sterilized, or sanitized.

FIG. 26F illustrates an embodiment of the schematic of FIG. 26A during a fourth Phase of the flow chart of FIG. 26B, wherein the system is cleared.

### DETAILED DESCRIPTION

### General

Sterilization, disinfection, sanitization, and decontamination methods are used in a broad range of applications, and have used an equally broad range of sterilization, disinfection, sanitization, and decontamination agents. The term “sterilization” generally refers to the inactivation of bio-contamination, especially on inanimate objects. The term “disinfection” generally refers to the inactivation of organisms considered pathogenic. Although the term “sterilization” may be used in describing certain embodiments herein, it would be appreciated that, unless otherwise indicated, such embodiments can also be used for disinfection (e.g., high-level disinfection, low-level disinfection, etc.), sanitization, and/or other types of decontamination, e.g., as provided with their regulatory definitions.

Pulsed or silent electric discharge in air or other gases produces non-thermal plasma. Non-thermal plasma processing involves producing plasma in which the majority of the electrical energy goes into the excitation of electrons. These plasmas are characterized by electrons with kinetic energies much higher than those of the ions or molecules. The electrons in these plasmas are short-lived under atmospheric pressure; instead, they undergo collisions with the preponderant gas molecules. The electron impact on gas molecules causes dissociation and ionization of these molecules, which creates a mix of reactive species, in the form of free radicals, reactive oxygen and nitrogen species, ions, and secondary electrons. These reactive species cause unique and diverse chemical reactions to occur, even at relatively low temperatures. These chemical reactions are utilized in low temperature decontamination and sterilization technologies. While there are certain non-thermal plasma devices for wound treatment (or disinfection, sterilization, etc. of devices and objects), prior to the embodiments disclosed herein, all suffered from various therapeutic and practical limitations. First, all of these devices require interaction between the plasma and the wound (or object); that is, since the electric discharge takes place directly on the tissue, the treated tissue itself plays the role of an electrode. This makes the application of non-thermal plasma exquisitely sensitive to small movements or changes in geometry. This adds significant complexity to the treatment and requires the provider to have specialized training to maintain the proper tolerances. Other limitations include the inability to cover large surface areas in a short period of time and equipment that has a large environmental footprint and requires a high upfront cost. Additionally, current commercialized non-thermal plasma devices have a requirement for significant provider training and time to administer treatment including one on one provider to patient care.

As discussed in detail herein, vaporized hydrogen peroxide (VHP) can be used for sterilization. Certain methods of sterilization with VHP include open loop systems, in which the VHP is applied to the items to be sterilized and then exhausted, and closed loop systems, where sterilizing vapors are recirculated.

In closed loop systems, a carrier gas, such as air, is dried and heated prior to flowing past a vaporizer. A hydrogen peroxide aqueous solution is introduced into the vaporizer and which enables this solution to be vaporized. The resulting vapor is then combined with the carrier gas and introduced into a sterilization chamber of varying size, shape, and material. A blower exhausts the carrier gas from the sterilization chamber and recirculates the carrier gas to the vaporizer where additional VHP is added. Between the sterilization chamber and the vaporizer, the recirculating carrier gas passes through a catalytic destroyer (where any remaining VHP is eliminated from the carrier gas), a dryer, a filter, and a heater.

United States Patent Application Publication No: US 2005/0129571 A1 by Centanni discloses a closed loop sterilization system. The purpose of using the closed loop is to prevent decrease of the free radical concentration in the circulating effluent. Centanni teaches that there should be a VHP (vapor hydrogen peroxide) destroyer employed in the loop. Centanni teaches that the ozone is mixed with the hydrogen peroxide vapor or microdroplets and the vapor or microdroplets are produced by injecting hydrogen peroxide water solution on a hot plate and thus evaporating it.

As discussed in greater detail herein the present application provides for various systems and related methods for sterilizing, disinfecting, sanitizing, and/or decontaminating a variety of items, ranging from surgical equipment or other medical devices to electronic equipment, as well as services, rooms, and other items including, but not limited to soft goods, foods, and related manufacturing equipment. A general overview will be provided, with additional detail related to each of the components of such systems provided below. As mentioned above, the term “sterilization” shall be appreciated to not only encompass the removal of all or substantially all microorganisms and or other pathogens from an object or surface but shall also encompass (unless otherwise specified) disinfection, sanitizing, and decontamination.

### Overview

The present application discusses concepts relating to removing and/or reducing the presence of viable microorganisms on a surface. This discussion is intended to cover concepts of sterilization, disinfection, sanitization, and decontamination. Decontamination is generally defined as killing some bacteria and fungi while deactivating viruses. Disinfection and sanitization are two levels of decontamination; “disinfection” refers to killing nearly 100% of germs on surfaces or objects while “sanitization” refers to lowering the number of microorganisms to a safe level by either cleaning or disinfecting. Sterilization, on the other hand, refers to the killing of all microorganisms, viruses, and bacterial spores. Each of these concepts refer to a different level of removing and/or reducing the viability of microorganisms on a surface. Unless specified otherwise, reference to a system or method for removing and/or reducing the presence of microorganisms on a surface is intended to encompass all level of reducing microbial burden/viability (e.g., disinfection, sanitization, decontamination, and sterilization).

The prevention of acquired infections, whether in a commercial, home, or healthcare setting, is an important concern. This can be particularly difficult during a viral outbreak when frequently used items must be regularly disinfected, sterilized, and/or sanitized to prevent spread the spread of the virus. Existing methods of disinfection, sterilization, and/or sanitization are inadequate or burdensome. For example, disinfectant wipes can be ineffective if contact time is insufficiently long, or proper protocols are not followed. As well, seams and irregular surfaces can be difficult to reach using manual methods of disinfection, sterilization, and or sanitization. UV systems may be capital intensive, may not be EPA or FDA registered, and may have difficulty treating resilient organisms such as spores. Harsh chemical can damage devices and the exposure of individuals to chemical disinfectants can cause health risks. Lastly, the use of disposable methods of disinfection, sterilization, and/or sanitization can cause a significant environmental impact.

Disclosed are devices, systems, and methods for reducing microorganisms on a surface. As will be discussed in more detail below, the device for reducing microorganisms on a surface can be a fully automated system that is intended to disinfect hard non-porous surfaces for reusable non-critical medical devices and general-use items used in healthcare facilities. The disclosed device provides for rapid and effective broad-spectrum disinfection of items used in various settings (e.g., patient care settings) that offer consistent disinfection for patients, healthcare workers, and equipment used in those settings. Although discussions of the use of the disclosed device may be focused predominantly on healthcare settings, the disclosed device can be intended for home, commercial, or field use.

In some embodiments, the disclosed device is configured to operate at ambient temperature and ambient pressure conditions in a continuous closed loop flow throughout the disinfection, sterilization, and/or sanitization process.

The device for reducing the viability of microorganisms on a surface includes a chamber for receiving the items for reducing the viability of microorganisms on a surface. In some examples, the device can include a chamber with a plurality of removable shelves on which items for disinfection can be placed.

The device for reducing the viability of microorganisms on a surface can include a 50% hydrogen peroxide as the active ingredient for reducing the viability of microorganisms on a surface. In some embodiments, the 50% hydrogen peroxide is packaged in cartridges that can be removed and replaced from the system when the hydrogen peroxide solution is consumed. In some examples, the hydrogen peroxide can be introduced into the system for reducing microorganisms on a surface using a nebulizer that is configured to convert the hydrogen peroxide solution into a micro-spray that inactivates the microorganisms. In some examples, the system can include an ozone generator that produces ozone to condition the system chamber prior to and after the disinfection, sterilization, and/or sanitization process.

The device for reducing the viability of microorganisms on a surface can be configured such that once the disinfection, sterilization, and/or sanitization is completed, fresh air is automatically introduced into the system through a HEPA filter to flush out the system chamber before the disinfected, sterilized, and/or sanitized items are removed. After the disinfection, sterilization, and/or sanitization process is completed, the air that exits the system chamber can be exhausted through a HEPA and an activated carbon filter to ensure substantially only or only clean air leaves the system.

In some embodiments, the device for reducing the viability of microorganisms on a surface can be a fully integrated system that includes hardware, electronics, and software to operate and monitor the system. The system can be programmed to automatically disinfect, sterilize, and/or sanitize the items placed in the device with the push of a button by the user.

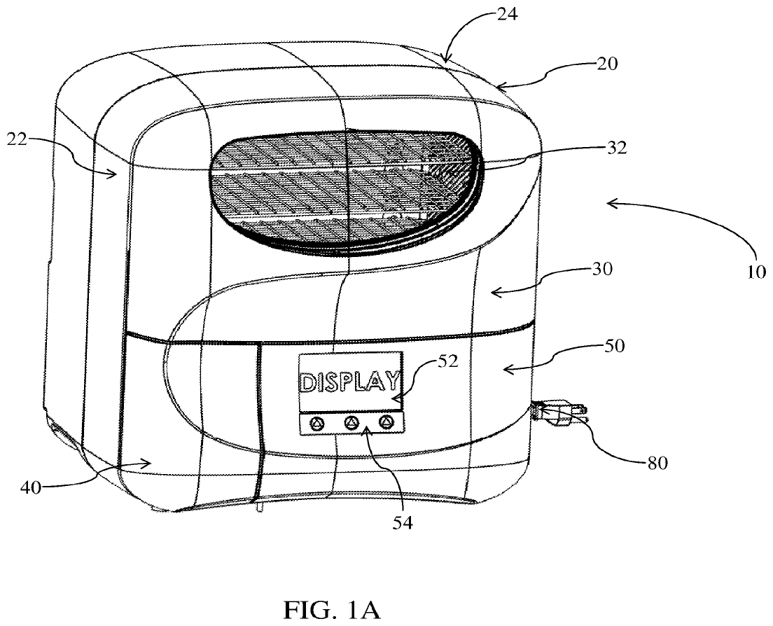

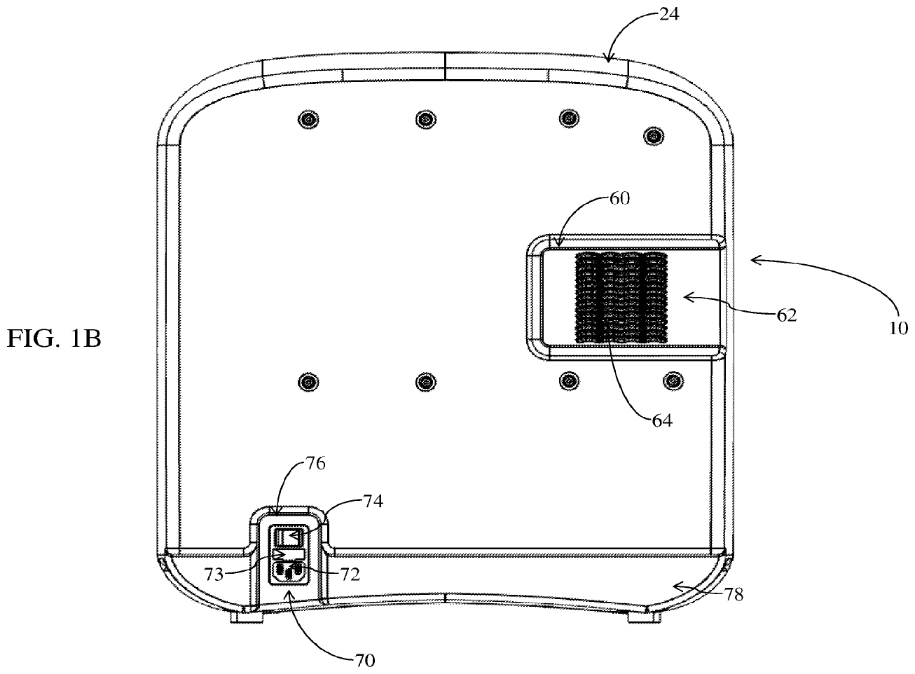

FIGS. 1A-1B illustrate perspective views of the front and rear of the device for disinfection, sterilization, and/or sanitization 10. In some embodiments, the device for disinfection, sterilization, and/or sanitization 10 includes a housing 20 with a front panel 22 and a rear panel 24. In some examples, the front panel 22 of the housing 20 can include a chamber door 30, a cartridge door 40, and a service door 50. As will be discussed in more detail below, the chamber door 30 can be used to open to allow access to the chamber 100. When closed, the chamber door 30 can seal the chamber 100. In some examples, the cartridge engagement mechanism 300 can include a window 32 that allows items placed in the chamber 100 to be visible to the user. In some embodiments, the cartridge door 40 can open to allow access to the cartridge engagement mechanism 300 and the cartridge 400. When the cartridge door 40 is disengaged, the user can access the cartridge engagement mechanism 300 to remove and/or replace the cartridge 400 in the cartridge engagement mechanism 300. In some examples, the service door 50 can disengage to allow access to the plurality of filters 230.

In some embodiments, the service door 50 can include a user display 52 and a plurality of buttons 54. In some embodiments, the user display 52 can provide information regarding the device for disinfection, sterilization, and/or sanitization 10 to the user. In some examples, the plurality of buttons 54 can allow the user to interact with the system of the device for disinfection, sterilization, and/or sanitization 10. For example, the user can begin, alter, and/or end the disinfection, sterilization, and/or sanitization process. In other examples, the user can diagnose problems with the device for disinfection, sterilization, and/or sanitization 10 and the user display 52 can provide the user information to fix any errors identified. In other examples, the user display 52 can provide information regarding the device and or the systems for disinfection, sterilization, and/or sterilization. In some embodiments, the user display 52 can be configured to provide information to the user on the status of the disinfection, sterilization, and/or sanitization of the items in the chamber 100.

FIG. 1B illustrates the rear panel 24 that can include an exhaust 60. In some embodiments, the exhaust 60 forms a recess 62 in the wall of the rear panel 24. As shown in FIG. 1B, a vent 64 of the exhaust 60 can be positioned on the rear panel 24 such that, if the device for disinfection, sterilization, and/or sanitization 10 is placed against a surface, the vent 64 can be set away from the wall. In some examples, the rear panel 24 can include a recess 76 for a power entry module 70 and a power cord recess 78. As shown in FIG. 1B, the power entry module 70 is further recessed in the rear panel 24 than the power cord recess 78. The arrangement of the recess 76 of the power entry module 70 and the power cord recess 78 can allow a power cord 80 to be engaged with the power entry module 70 and wrap around the device while allowing the rear panel 24 to be positioned flush against a wall. In some embodiments, the power cord recess 78 can extend across the entire length of the rear panel 24 to allow the power cord 80 to be arranged along either side of the device 10. In some examples, the power entry module 70 can include a power jack 72, a power switch 74, and a fuse 73. As mentioned above, the position of the power entry module 70 in the recess 76 allows the power switch 74, fuse 73, and power jack 72 to be set away from a wall if the device 10 is set against a wall.

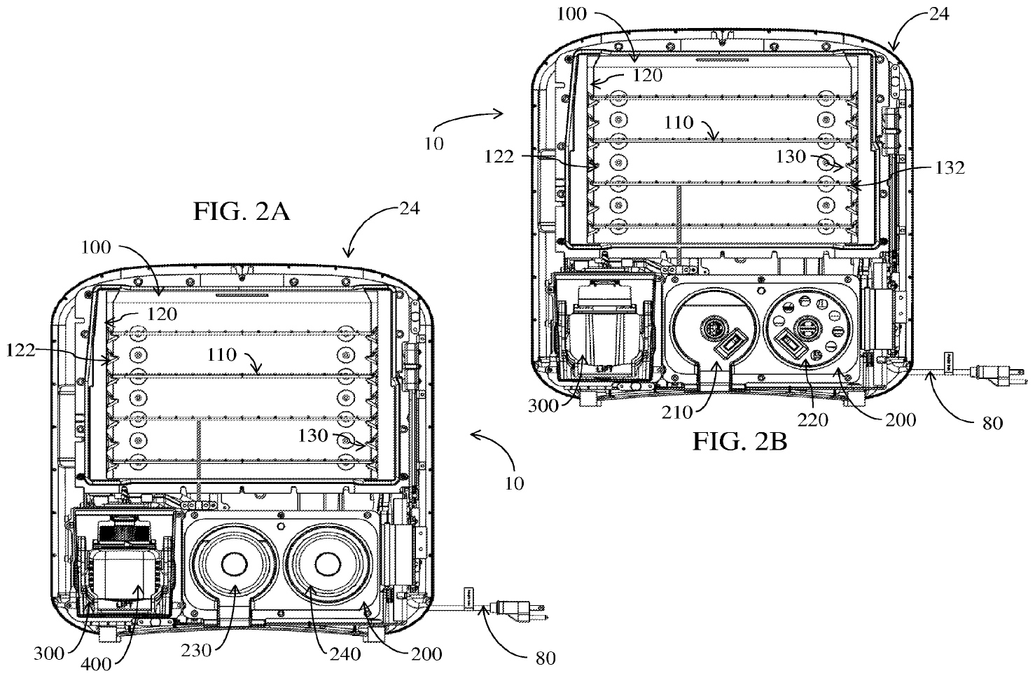

FIGS. 2A-2B illustrate a front view of the device 10 with the front panel 22 removed. With the front panel 22 removed, the chamber 100, the cartridge engagement mechanism 300, and the filter holder 200 are visible. FIG. 2A illustrates the device 10 with the cartridge 400 positioned in the cartridge engagement mechanism 300 and the filters 230, 240 positioned in the filter holder 200. FIG. 2B illustrates the device 10 with the cartridge 400 removed from the cartridge engagement mechanism 300 and the filters 230, 240 removed from the filter holder 200.

As will be discussed in more detail below, the device 10 can include a plurality of sensors such that the system can include a number of self-diagnostic features. As will be discussed, if any of the critical components are non-functioning, the user display 52 can provide an error message that will indicate to the user that something in the system is malfunctioning. In some embodiments, the user display 52 can help the user diagnose the problem with the system and to find ways to address the error.

### Chamber

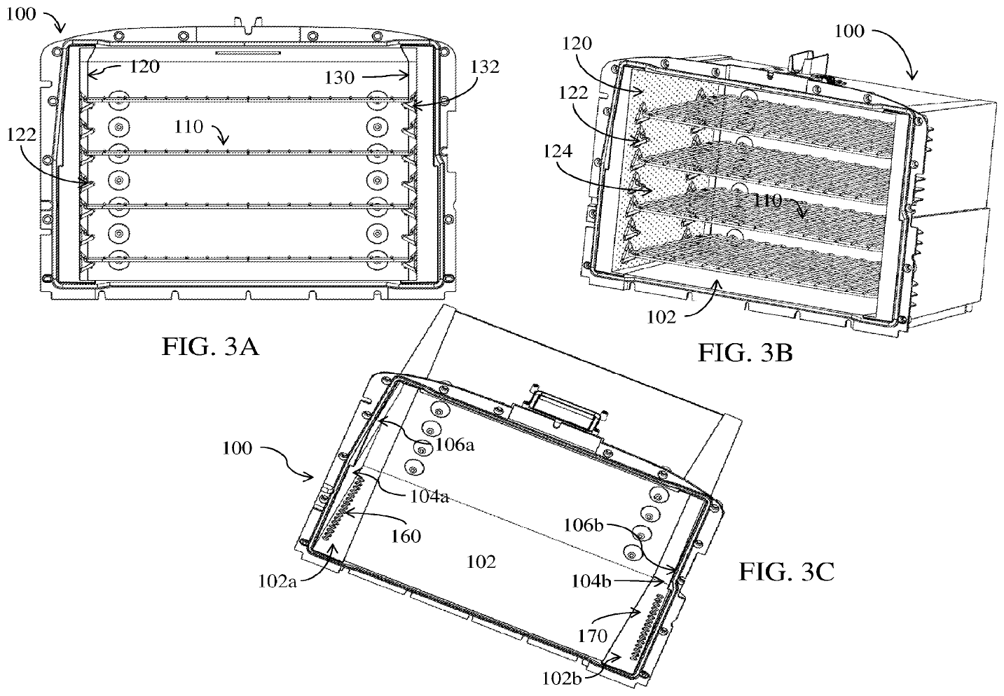

FIGS. 3A-3C illustrate a plurality of views of the chamber 100. As shown in FIGS. 3A-3B, the chamber 100 can include an inlet plate 120 and an outlet plate 130. In some embodiments, the inlet plate 120 can include a plurality of securement hooks 122 and the outlet plate 130 can include a plurality of securement hooks 132. As shown in FIGS. 3A-3B, each of the plurality of securement hooks 122 and the securement hooks 132 can secure one or a plurality of wire racks 110. In some examples, each of the inlet plate 120 and the outlet plate 130 include a plurality of openings 124 and openings 134 that are configured to provide for fluid flow to enter and exit the chamber 100.

FIG. 3C illustrates the chamber 100 with the inlet plate 120, the outlet plate 130, and the plurality of wire racks 110 removed. As shown, the chamber 100 can include a base 102, a first wall 104a, and a second wall 104b. In some embodiments, the base 102 can include a first recess 102a on a first side of the base 102 and a second recess 102b on a second side of the base 102. In some embodiments, the first recess 102a can form a recess in the first side of the base 102 and include a plurality of inlet openings 160. The inlet openings 160 can allow fluid flow into the chamber 100. In some examples, the first wall 104a includes a first angled portion 106a that is configured to engage the inlet plate 120. In some examples, the shape of the first wall 104a is structured to create a minimal pressure drop behind the first wall 104a (e.g., between the wall of the chamber 100 and the first wall 104a). This can help to provide a uniform, or substantially uniform airflow through the chamber 100. In some embodiments, the shape and position of the first angled portion 106a can help to direct the fluid flow from the inlet openings 160 and uniformly through the openings 124 of the inlet plate 120. In some examples, the second recess 102b is similar to the first recess 102a and can form a recess in the second side of the base 102. In some embodiments, the first recess 102a can include a plurality of outlet openings 170. The outlet openings 170 can allow fluid flow to exit the chamber 100. In some embodiments, the second wall 104b includes a second angled portion 106b that is configured to engage the outlet plate 130. In some examples, like the first angled portion 106a, the shape and position of the second angled portion 106b can help to direct the fluid flow out of the chamber 100 through the outlet plate 130 and out of the outlet openings 170. In some examples, the shape of the second wall 104b is structured to create a minimal pressure drop behind the second wall 104b (e.g., between the wall of the chamber 100 and the 140b). This can help to provide uniform airflow through the chamber 100.

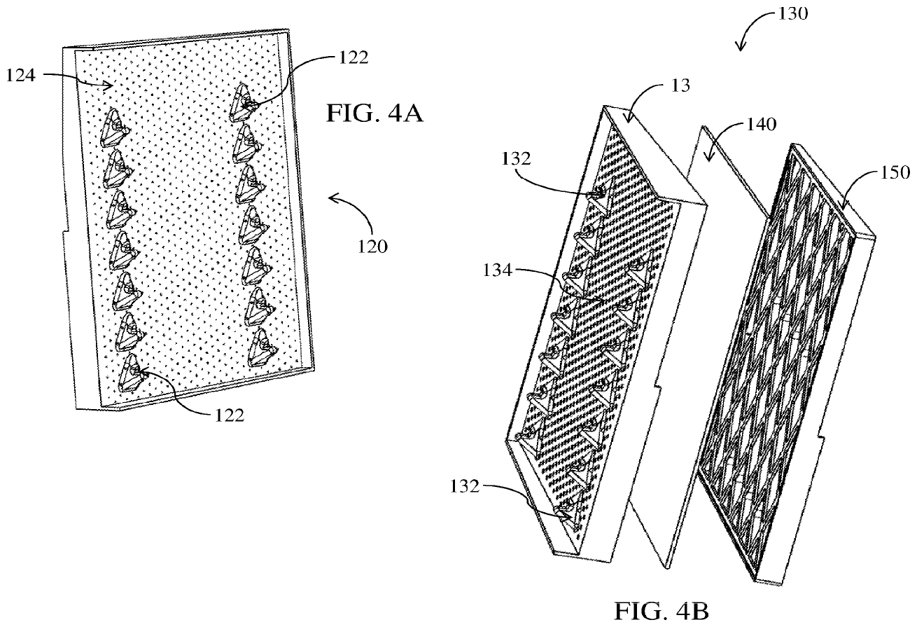

FIGS. 4A and 4B illustrate perspective views of the inlet plate 120 and the outlet plate 130. As discussed above, the inlet plate 120 can include a plurality of securement hooks 122 for securing a first side of the plurality of wire racks 110. In some embodiments, the inlet plate 120 can include a plurality of openings 124 to allow the fluid flow to pass uniformly through the inlet plate 120 and into the chamber 100. In some embodiments, the inlet plate 120 can include more than 1,000 openings, more than 1,100 openings, more than 1,200 openings, more than 1,300 openings, more than 1,400 openings, more than 1,500 openings, between 1,000-1,100 openings, between 1,100-1,200 openings, between 1,200-1,300 openings, between 1,300-1,400 openings, between 1,400-1,500 openings, and any value in between those ranges listed, including endpoints. In some embodiments, each of the openings 124 can have a diameter of approximately 0.50 mm, of approximately 0.60 mm, of approximately 0.70 mm, of approximately 0.80 mm, of approximately 0.90 mm, of approximately 1.0 mm, of approximately 1.10 mm, of approximately 1.20 mm, of approximately 1.30 mm, of approximately 1.40 mm, of approximately 1.50 mm, of between 0.50-0.60 mm, of between 0.60-0.70 mm, of between 0.70-0.80 mm, of between 0.80-0.90 mm, of between 0.90-1.0 mm, of between 1.0-1.10 mm, of between 1.10-1.20 mm, of between 1.20-1.30 mm, of between 1.30-1.40 mm, of between 1.40-1.50 mm, and any value in between those ranges listed, including endpoints. The outlet plate 130 is configured to allow fluid flow to pass through and exit the chamber 100 through the outlet openings 170. As shown in FIG. 4B, the body 131 of the outlet plate 130 can be similar to the inlet plate 120. The body 131 can include a plurality of securement hooks 132 for securing a second side of the wire racks 110. In some embodiments, the body 131 of the outlet plate 130 can include a plurality of openings 134 to allow the fluid flow to pass uniformly through the outlet plate 130 and out of the chamber 100. In some embodiments, the inlet plate 120 can include more than 1,000 openings, more than 1,100 openings, more than 1,200 openings, more than 1,300 openings, more than 1,400 openings, more than 1,500 openings, more than 1,600 openings, more than 1,700 openings, more than 1,800 openings, more than 1,900 openings, more than 2,000 openings, between 1,000-1,100 openings, between 1,100-1,200 openings, between 1,200-1,300 openings, between 1,300-1,400 openings, between 1,400-1,500 openings, between 1,500-1,600 openings, between 1,600-1,700 openings, between 1,700-1,800 openings, between 1,800-1,900 openings, between 1,900-2,000 openings, and any value in between those ranges listed, including endpoints. In some embodiments, each of the openings 134 can have a diameter of approximately 1.0 mm, of approximately 1.10 mm, of approximately 1.20 mm, of approximately 1.30 mm, of approximately 1.40 mm, of approximately 1.50 mm, of approximately 1.60 mm, of approximately 1.70 mm, of approximately 1.80 mm, of approximately 1.90 mm, of approximately 2.0 mm, of approximately 2.10 mm, of approximately 2.20 mm, of approximately 2.30 mm, of approximately 2.40 mm, of approximately 2.50 mm, of between 1.0-1.10 mm, of between 1.10-1.20 mm, of between 1.20-1.30 mm, of between 1.30-1.40 mm, of between 1.40-1.50 mm, of between 1.50-1.60 mm, of between 1.60-1.70 mm, of between 1.70-1.80 mm, of between 1.80-1.90 mm, of between 1.90-2.0 mm, of between 2.0-2.10 mm, of between 2.10-2.20 mm, of between 2.20-2.30 mm, of between 2.30-2.40 mm, of between 2.40-2.50 mm, and any value in between those ranges listed, including endpoints. In some embodiments, the outlet plate 130 can include a body 131, a filter 140, and a filter support 150 for securing the filter 140. In some examples, the filter 140 can be configured to prevent impurities from circulating through the rest of the system of the device 10. In some embodiments, the filter 140 can filter out large dust particles that can contaminate the other components in the system 15 (e.g., the ozone generator 740) and reduce the efficiency of the system 15. The filter 140 of the outlet plate 130 can be secured in place by the filter support 150

As discussed above, the airflow through the chamber 100 is uniform. This is possible for a number of reasons. In some examples, the size of the openings 124 in the inlet plate 120 and the size of the openings 134 in the outlet plate 130 are configured to restrict airflow slightly which can force air to flow more equally through all of the openings 124 and openings 134. In some embodiments, the size of the openings 124 and openings 134 are of a size that provide more restriction than pressure than the dynamic pressure of the airflow in the space behind the inlet plate 120 and the outlet plate 130. In some examples, the large number of openings 124 and openings 134 on the inlet plate 120 and outlet plate 130 respectively can help to create an even distribution of air flow through each of the inlet plate 120 and the outlet plate 130 from top to bottom and front to back. In some embodiments, the plurality of securement hooks 122 on the inlet plate 120 and the plurality of securement hooks 132 on the outlet plate 130 are positioned so as not to interrupt the pattern formed by the openings 124 and the openings 134 on the inlet plate 120 and the outlet plate 130 respectively. In some embodiments, the plurality of inlet openings 160 at the first recess 102a and the plurality of outlet openings 170 at the second recess 102b can also aid in the even distribution of airflow from the front to the back of the chamber 100. In some embodiments, the filter 140 can help to ensure a uniform airflow through the chamber 100 by restricting airflow.

In some embodiments, the airflow setup within the system provides a device 10 at a slight vacuum. In the event that any leaks exist in the system, fluid flows into the airway flow system and any excess air is forced through the exhaust filter. In some examples, the device 10 measures the amount of air that is exiting as a measure of health of the system. In some embodiments, the device 10 provides for laminar airflow through the system. The geometry of each of the panels (e.g., the inlet plate 120 and the outlet plate 130), the feeds, and the perforation (e.g., the openings 124 of the inlet plate 120 and the openings 134 of the outlet plate 130) ensure that there is very uniform through the chamber 100.

In some embodiments, the rate and passage of air can be approximately 10 air exchanges/minute, approximately 12 air exchanges/minute, approximately 14 air exchanges/minute, approximately 16 air exchanges/minute, approximately 18 air exchanges/minute, approximately 20 air exchanges/minute, approximately 22 air exchanges/minute, approximately 24 air exchanges/minute, approximately 26 air exchanges/minute, approximately 28 air exchanges/minute, approximately 30 air exchanges/minute, between approximately 10 to 15 air exchanges/minute, between approximately 15 to 20 air exchanges/minute, between approximately 15 to 20 air exchanges/minute, between approximately 20 to 25 air exchanges/minute, between approximately 25 to 30 air exchanges/minute, and any value in between those ranges listed, including endpoints.

In some examples, the rate and passage of air can be about 200 L/min., about 210 L/min., about 215 L/min., about 220 L/min., about 225 L/min., about 230 L/min., about 235 L/min., about 240 L/min., about 245 L/min., about 250 L/min., about 255 L/min., about 260 L/min., about 265 L/min., about 270 L/min., about 275 L/min., about 280 L/min., about 285 L/min., about 290 L/min., about 295 L/min., about 300 L/min., about 305 L/min., about 310 L/min., about 315 L/min., about 320 L/min., about 325 L/min., about 330 L/min., about 335 L/min., about 340 L/min., about 345 L/min., about 350 L/min., about 355 L/min., about 360 L/min., about 365 L/min., about 370 L/min., about 375 L/min., about 380 L/min., about 385 L/min., about 390 L/min., about 395 L/min., about 400 L/min., between about 200 to 210 L/min., between about 210 to 220 L/min., between about 220 to 230 L/min., between about 230 to 240 L/min., between about 240 to 250 L/min., between about 250 to 260 L/min., between about 260 to 270 L/min., between about 270 to 280 L/min., between about 280 to 290 L/min., between about 290 to 300 L/min., between about 300 to 310 L/min., between about 310 to 320 L/min., between about 320 to 330 L/min., between about 330 to 340 L/min., between about 340 to 350 L/min., between about 350 to 360 L/min., between about 360 to 370 L/min., between about 370 to 380 L/min., between 380 to 390 L/min., between about 390 to 400 L/min., and any value in between those ranges listed, including endpoints.

### Filters

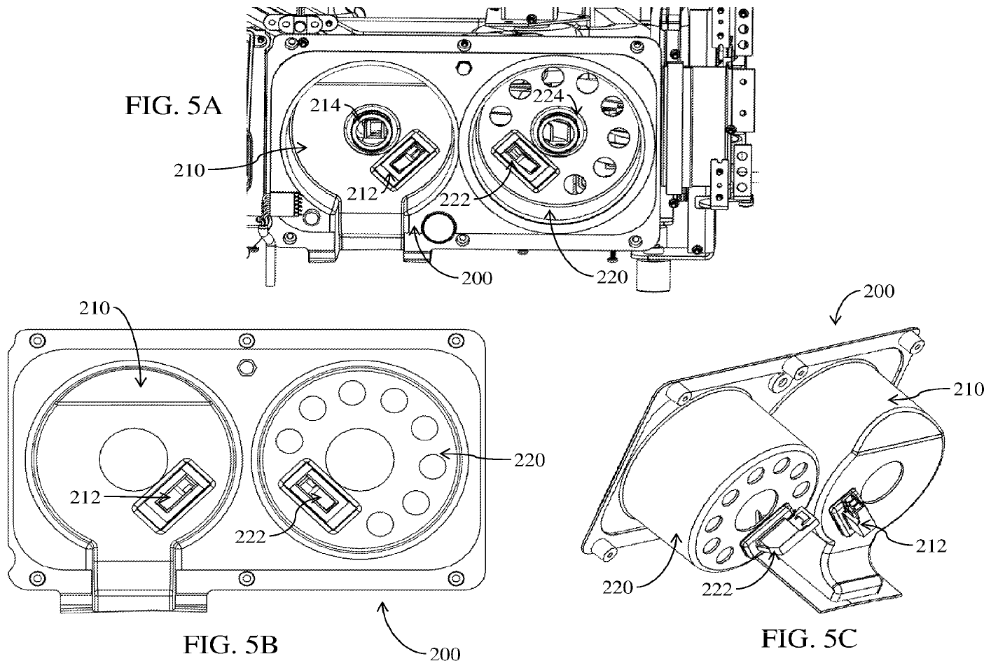

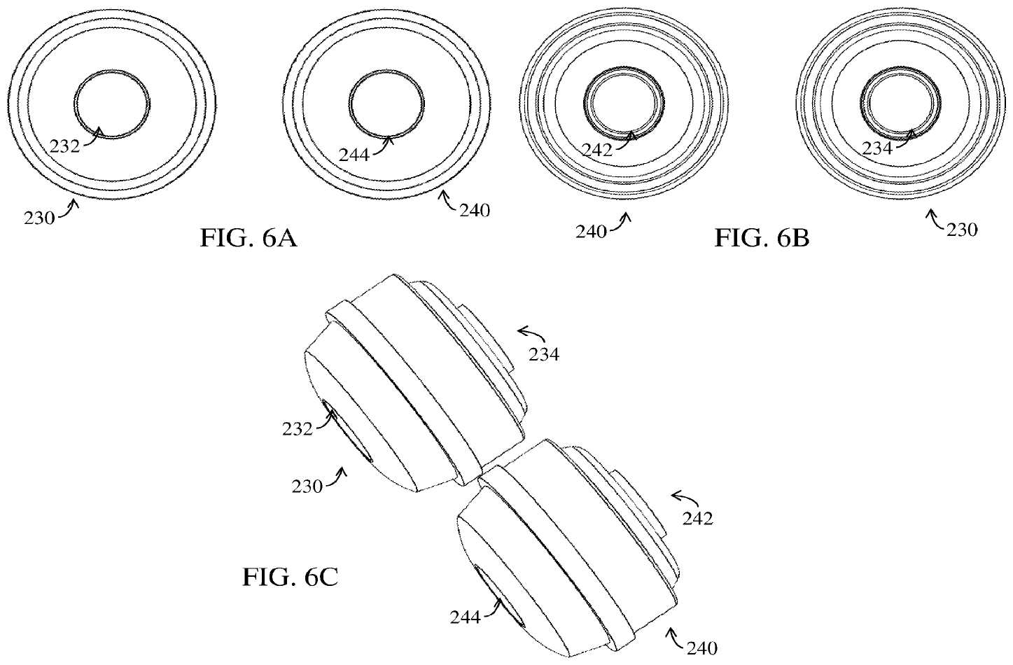

FIGS. 5A-5C illustrate an embodiment of a filter holder 200 positioned within the front portion of the device 10. FIGS. 6A-6C illustrate a plurality of views of the inlet filter 230 and the outlet filter 240.

Turning first to the filter holder 200, in some embodiments, the filter holder 200 includes a recess 210 for retaining the inlet filter 230 and a recess 220 for retaining the outlet filter 240. The filter holder 200 can be configured to provide an enclosure for each of the filters 230, 240 so as to separate the filters 230, 240 from the rest of the system 15 within the device 10. This can allow a user to access the filter holder 200 and change each of the filter 230 and the filter 240 while sealing off the rest of the system 15. In some examples, the filter holder 200 captures air from outside the device 10 and routes it to the inlet filter 230. In some embodiments, the recess 210 and the recess 220 are circular to retain the cylindrical filters 230, 240, however the recess 210 and the recess 220 can include any size or shape necessary to retain the appropriate filters. Each of the recess 210 and the recess 220 can include a respective adaptor 214 and adaptor 224 for securing the associated filter 230 and filter 240. Each of the recess 210 and the recess 220 can include an actuator 212 and actuator 222 that are connected to a sensor (not shown). In some embodiments, the actuator 212 and the actuator 222 are engaged when the respective filter (e.g., filter 230 and filter 240) are properly positioned. If the filter is not properly positioned, the associated actuator 212 and actuator 222 will not be engaged and the sensor will receive a signal that the filter is not properly engaged. The sensor will notify the system and will provide the user with an indicator via the user display 52. In some embodiments, the user will be unable to proceed with disinfection and/or sterilization before addressing the improperly placed/missing inlet and/or outlet filter 230, 240. The proper positioning of the filter—whether the inlet filter 230 or the filter 240—can be important to ensure the proper functioning of the device and also to prevent unsafe levels of disinfectant/sterilant from leaving the device 10.

FIGS. 6A-6C illustrate an embodiment of filters that can be secured in the filter holder 200 of the device 10. A variety of filter types can be used, depending on the embodiment. In some embodiments, the inlet filter 230 and the outlet filter 240 can be a HEPA filter and/or a carbon filter. In some embodiments the HEPA filter is configured to only allow things through less than 0.3 μm particle size. In some embodiments, ionic filters, carbon filters, UV filters, cellulose filters, silica-based filters, or the like are used, either alone or in combination. In some embodiments, the inlet filter 230 can be configured to filter environmental air and allow it to pass into the system. In some examples, the inlet filter 230 and the outlet filter 240 are the same. In other examples, the inlet filter 230 and the outlet filter 240 can be different types of filters. The inlet filter 230 can include an inlet 232 and an outlet 234. Similarly, the outlet filter 240 can include an inlet 242 and an outlet 244.

The inlet filter 230 draws in air from the surrounding area. When the disinfecting/sterilization process ends within the device 10, the device 10 conducts a purging process to clear out all the chemicals from the chamber 100 and throughout the system. During this process, room air is drawn in through the inlet filter 230 and flows throughout the system. The air is then blown through the inlet 242 of the filter 240 and out of the outlet 244 to ensure that only clean air comes out of the system of the device 10.

However, as will be discussed in more detail below, air blown out of the filter 240 is not directly discharged out of the device 10. Instead, it is pushed past a separate ambient ozone sensor before it is exhausted. As detailed further below, this provides a secondary check to prevent the device 10 from continuing to operate when unsafe levels of disinfectant/sterilant could potentially leave the device 10. For example, if there is a failure in the exhaust filter 240, the ambient ozone sensor will be able to detect unsafe levels of disinfectant/sterilant (e.g., ozone) in the air flow and provide the user with an error message. In some embodiments, when the ambient ozone sensor detects unsafe levels of disinfectant/sterilant (e.g., ozone), the device 10 will be unable to continue operation until the device 10 is repaired.

### Cartridge Engagement Mechanism

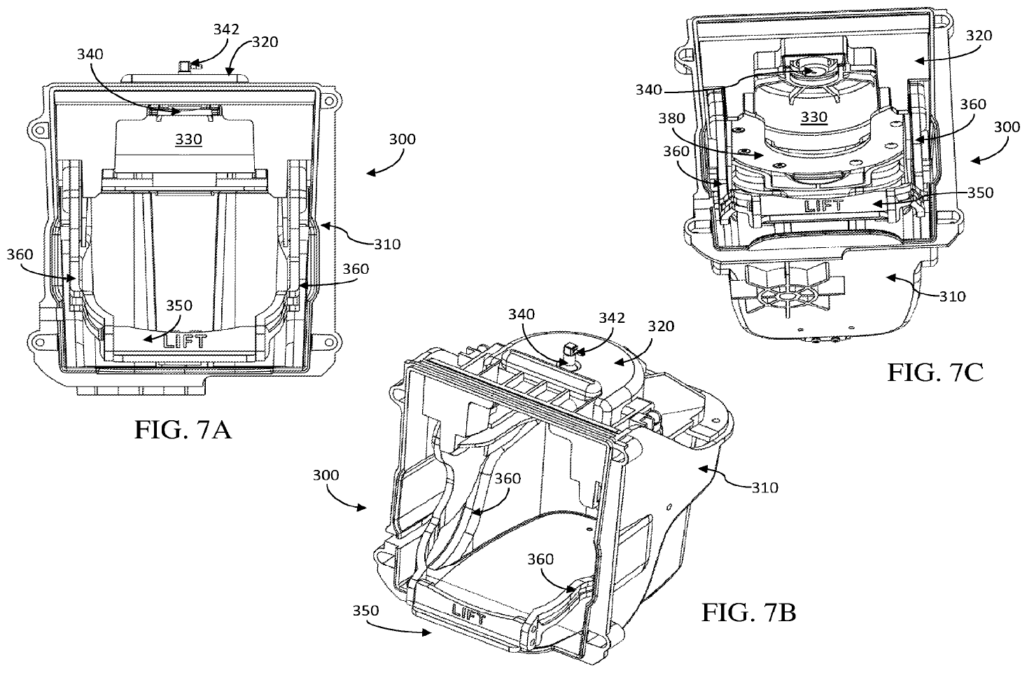

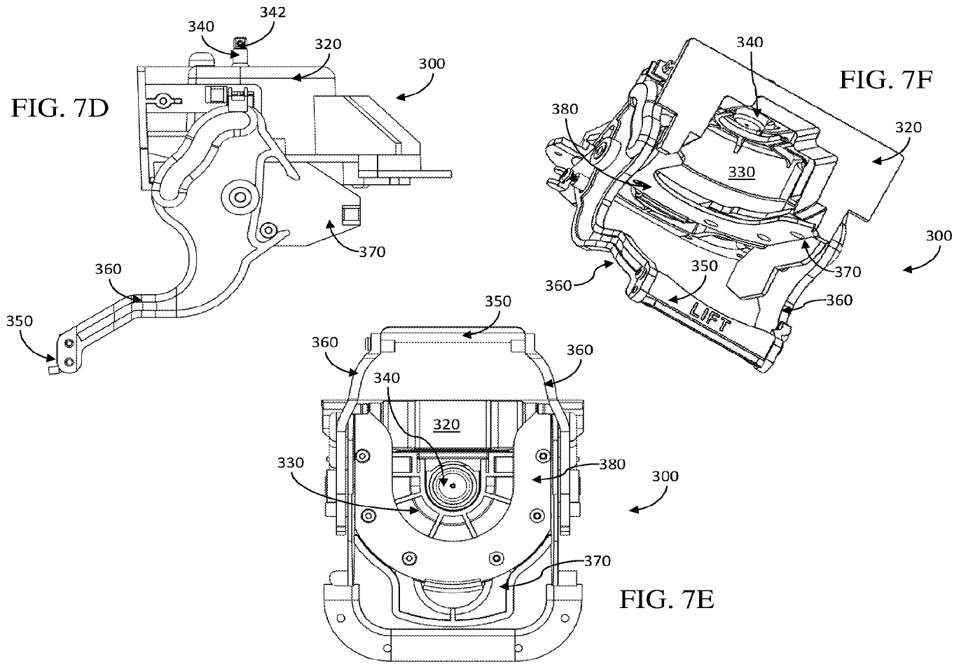

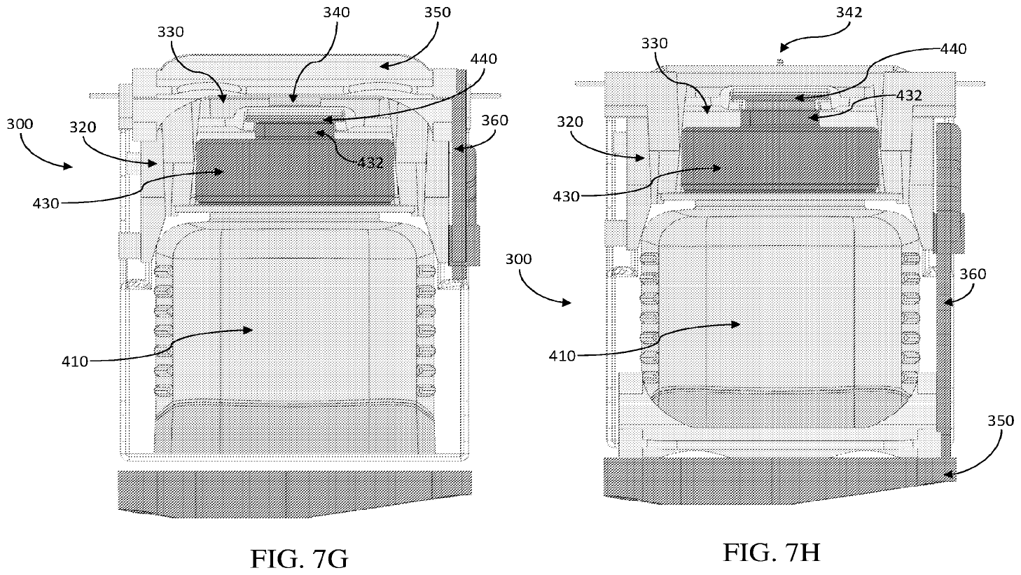

FIGS. 7A-7C illustrates an embodiment of a cartridge engagement mechanism 300 for securing a cartridge 400 in the device 10. As shown in FIGS. 2A-2B, the cartridge engagement mechanism 300 is positioned within the front portion of the device 10 and is positioned behind the cartridge door 40. FIGS. 7D-7E illustrates the cartridge engagement mechanism 300 in FIGS. 7A-7C with the lower frame 310 removed.

As shown, in some embodiments, the cartridge engagement mechanism 300 includes a lower frame 310 and an upper frame 320. The upper frame 320 can include a handle 350 and a plurality of arms 360 that are secured to the bracket and arm pivot 370. As will be discussed in more detail below, the handle 350 and the arms 360 can be moved from a first position to a second position to engage and disengage a spout of the cartridge 400. As shown in FIGS. 7E-7F, the underside of the upper frame 320 includes a spout lifter 330 that engages the spout 440 of the cartridge 400 with the flow seal 340 of the upper frame 320 to allow disinfectant and/or sterilant to be sucked out of the cartridge 400. In some embodiments, the flow seal 340 is fluidly connected to the flow fitting 342 that extends from the top of the upper frame 320. As will be discussed in more detail below, the flow seal 340 can be fluidly connected to the spout 440 such that fluid can flow out of the cartridge 400. In some examples, the upper frame 320 includes a shoulder 380 that can secure a transfer collar 454 of the cartridge 400 to prevent the cartridge 400 from moving as the spout 440 is moved from an open to a closed configuration by the cartridge 400.

The lower frame 310 is positioned about the upper frame 320 to allow the cartridge 400 to be placed in the cartridge engagement mechanism 300. In some embodiments, the upper frame 320 is secured on the cartridge engagement mechanism 300 to still allow the plurality of arms 360 and the handle 350 to move between a first and a second position.

FIGS. 7G-7I illustrates an embodiment of the cartridge engagement mechanism 300 engaging with the cartridge 400. Cartridge engagement mechanism 300 can be configured to open and close the bottle 410 of the cartridge 400. When the bottle 410 is opened, the cartridge engagement mechanism 300 can allow suction of fluid from the bottle 410 into the rest of the system for disinfection/sterilization. FIG. 7G illustrates the cartridge engagement mechanism 300 when the spout 440 in the closure 430 is in a closed position. FIG. 7H illustrates the cartridge engagement mechanism 300 when the spout 440 in the closure 430 is in an opened position. The cartridge engagement mechanism 300 can include a lower frame 310 with a spout lifter 330 that is configured to engage the spout 440 of the cartridge 400.

The cartridge engagement mechanism 300 can include a handle 350 and a plurality of arms 360 that can move spout lifter 330 in the cartridge engagement mechanism 300 between a closed and an opened position. The cartridge engagement mechanism 300 can include a flow seal 340 with a flow fitting 342 that can allow be fluidly connected to the spout 440 such that fluid can flow out of the cartridge 400. As shown in FIG. 7G, the spout lifter 330 can be secured to the first end 444 of the spout 440. The spout lifter 330 can engage with the portion of the first end 444 that extends past the receiving portion 432. The handle 350 and the plurality of arms 360 can be in a first raised position while the spout 440 is in the closed position. As shown in FIG. 7G, a gap exists between the spout 440 and the flow seal 340.

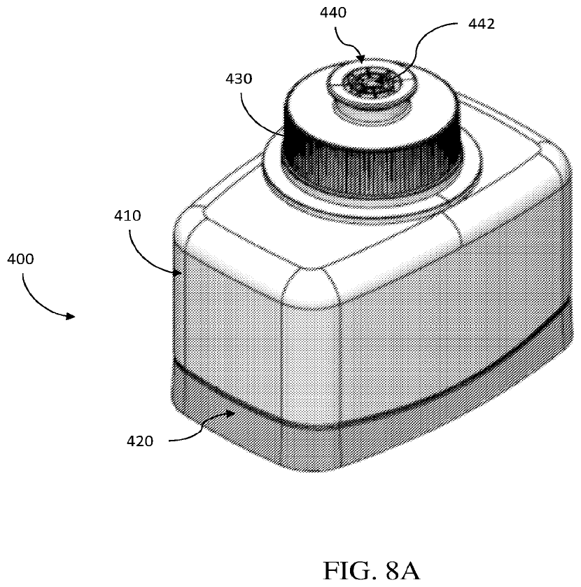

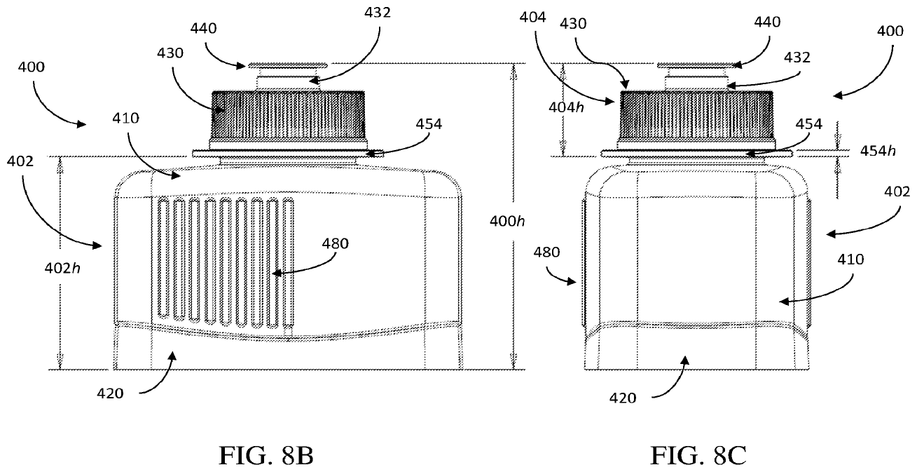

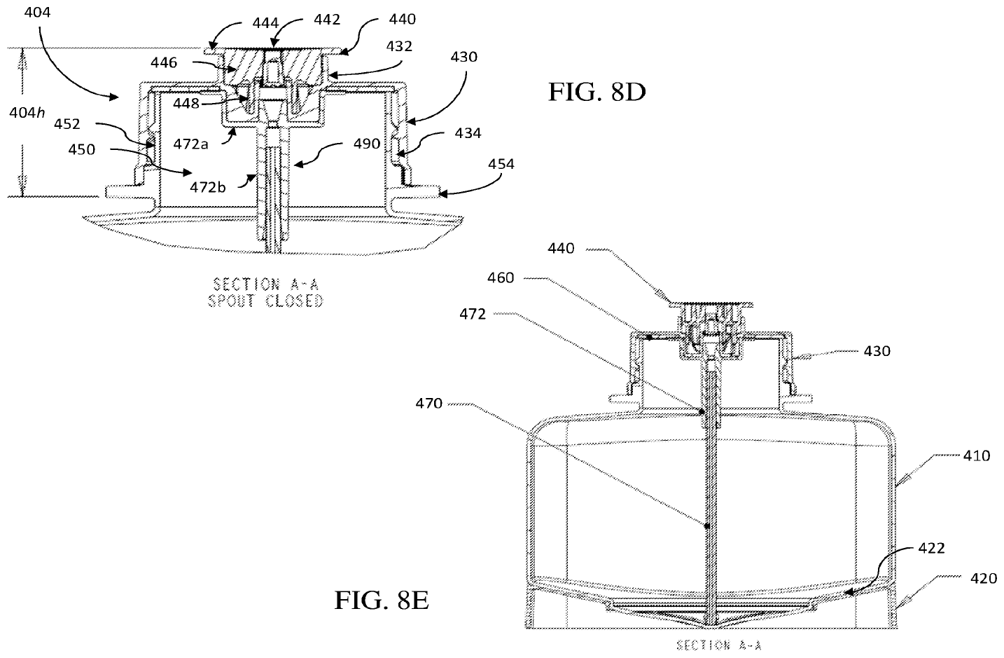

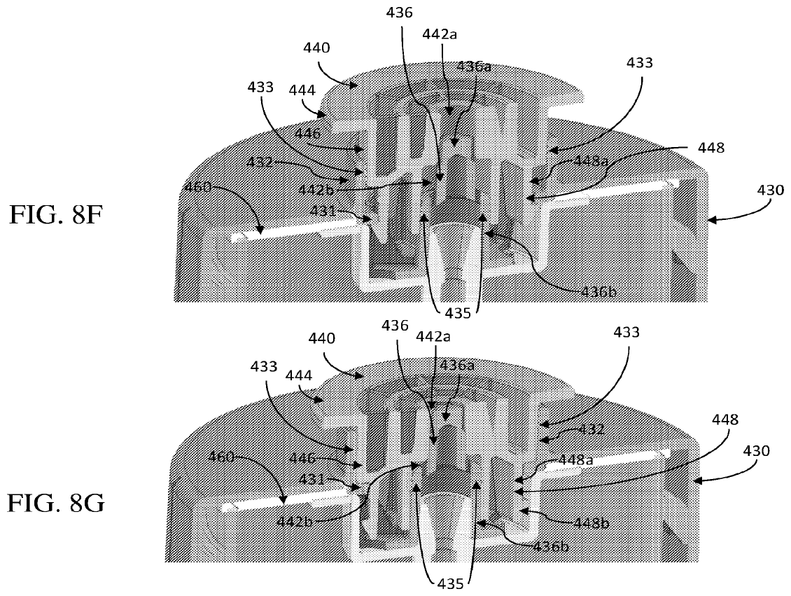

To move the spout 440 into an opened position, the handle 350 can be actuated and moved into a second lowered position as illustrated in FIG. 7H. A camming surface on the arms 360 can drive the spout lifter 330 upward. The spout lifter 330 unseats the spout 440 from the closure 430 and seals it against the flow seal 340. As shown in FIG. 7H, as the handle 350 is actuated, the spout lifter 330 moves upwardly to lift the spout 440 out of the receiving portion 432. As shown below in FIG. 8G, lifting the spout 440 out of the receiving portion 432, shifts the proximal end 436a of the conical seat 436 into the distal end 442b of the channel of the opening 442. The proximal end 436a can be cone shaped to allow fluid to flow around the proximal end 436a and out of the opening 442. In some embodiments, when the spout 440 is moved out of the receiving portion 432 and into an open position, the spout 440 is moved adjacent to a bottom surface of the flow seal 340 to seal the spout 440 against the flow seal 340. This can allow the spout 440 to be fluidly connected to the flow seal 340 to allow fluid to flow out of the cartridge 400 and out of the cartridge engagement mechanism 300 through the flow fitting 342.

### Cartridge

FIGS. 8A-8E illustrate embodiments of the cartridge 400. In some embodiments, the cartridge 400 can be replaceable by the user. In some examples, the cartridge 400 contains the disinfectant/sterilant that is used by the system for reducing microorganisms on a surface to disinfect, sterilize, and/or sanitize an item placed in the chamber 100. In some embodiments, the cartridge 400 can contain a hydrogen peroxide solution. In particular, the cartridge 400 contains a 50% hydrogen peroxide solution. In some embodiments, the cartridge 400 can hold a volume between system 600 mL and 660 mL. In some examples, the cartridge 400 can hold a volume of disinfectant/sterilant that is 600 mL, 605 mL, 610 mL, 615 mL, 620 mL, 625 mL, 630 mL, 635 mL, 640 mL, 645 mL, 650 mL, 660 mL between 600 mL and 605 mL, between 605 mL and 610 mL, between 610 and 615 mL, between 615 mL and 620 mL, between 620 mL and 625 mL, between 625 mL and 630 mL, between 630 mL and 635 mL, between 635 mL and 640 mL, between 640 mL and 645 mL, between 645 mL and 650 mL, between 650 mL and 655 mL, and between 655 mL and 660 mL.

FIGS. 8A-8E illustrate an embodiment of the cartridge 400 in more detail. The cartridge 400 can include a body 402 and a closure 430 that engages with a proximal end of the body 402 to allow the disinfectant/sterilant in the body of the cartridge 400 to be dispensed into the system of the device 10. In some embodiments, the cartridge 400 has a height 400h of between 4.0-5.0 inches. In some embodiments, the height 400h is 4.0 inches, 4.1 inches, 4.2 inches, 4.3 inches, 4.4 inches, 4.5 inches, 4.6 inches, 4.7 inches, 4.8 inches, 4.9 inches, 5.0 inches or between about 4.0-4.1 inches, between about 4.1-4.2 inches, between about 4.2-4.3 inches, between about 4.3-4.4 inches, between about 4.4-4.5 inches, between about 4.5-4.6 inches, between about 4.6-4.7 inches, between about 4.7-4.8 inches, between about 4.8-4.9 inches, or between 4.9-5.0 inches. In some embodiments, the body 402 has a height 402h of between 3.0-4.0 inches. In some embodiments, the height 402h is 3.0 inches, 3.05 inches, 3.10 inches, 3.15 inches, 3.20 inches, 3.25 inches, 3.30 inches, 3.35 inches, 3.40 inches, 3.45 inches, 3.50 inches, 3.55 inches, 3.60 inches, 3.65 inches, 3.70 inches, 3.75 inches, 3.80 inches, 3.85 inches, 3.90 inches, 3.95 inches, 4.0 inches or between 3.0-3.10 inches, between 3.10-3.20 inches, between 3.20-3.30 inches, between 3.30-3.40 inches, between 3.40-3.50 inches, between 3.50-3.60 inches, between 3.60-3.70 inches, between 3.70-3.80 inches, between 3.80-3.90 inches, and between 3.90-4.0 inches.

In some examples, the body 402 of the cartridge 400 can include a bottle 410 and a bottle stand 420. As illustrated in the cross-sectional view of the cartridge 400 in FIG. 8E, the bottle stand 420 can include an opening 422 on the proximal end of the bottle stand 420 that is configured to receive and stabilize a distal end of the bottle 410. In some embodiments, the opening 422 has a cross-section that forms a taper and the distal end of the bottle 410 forms a corresponding taper. In some examples, the bottle 410 and bottle stand 420 are attached. In some embodiments, the bottle 410 and the bottle stand 420 are separate components that can engage and be secured with each other.

The bottle 410 can be configured to store and provide a volume of disinfectant/sterilant for the system for reducing microorganisms on a surface of the items placed in the chamber 100. In some embodiments, the outer surface of the bottle 410 can include a ribbed feature 480. The ribbed feature 480 can allow the bottle 410 to be more easily gripped by the user. In some embodiments, the ribbed feature 480 can be located on at least one outside surface of the bottle 410. In some examples, the ribbed feature 480 can be located on opposite sides on the outside surface of the bottle 410. The bottle 410 can include a neck 450 on a proximal end of the bottle 410. The neck 450 can form an opening to the bottle 410 and can have a smaller diameter than the diameter of the bottle 410. In some examples, the neck 450 can include a transfer collar 454 that is disposed about the outer surface of the neck 450. The neck 450 can be configured to allow the cartridge 400 to be retained within the chamber 100 of the device 10. In some embodiments, the transfer collar 454 can have a shoulder thickness 454h of between 0-0.5 inches. In some examples, the shoulder thickness 454h is 0 inches, 0.05 inches, 0.10 inches, 0.15 inches, 0.20 inches, 0.25 inches, 0.30 inches, 0.35 inches, 0.40 inches, 0.45 inches, 0.50 inches or between 0-0.05 inches, between 0.05-0.10 inches, between 0.10-0.15 inches, between 0.15-0.20 inches, between 0.20-0.25 inches, between 0.25-0.30 inches, between 0.30-0.35 inches, between 0.35-0.40 inches, between 0.40-0.45 inches, or between 0.45-0.50 inches.

The cartridge 400 can include a closure 430 that is configured to engage with the neck 450 of the bottle 410. The closure 430 can include a threading 434 that is configured to threadingly engage with a threading 452 on the neck 450 of the bottle 410. The exterior surface of the closure 430 can include a plurality of ridges that allow the closure 430 to be more easily gripped by the user. The closure 430 can include a receiving portion 432 on a proximal end of the closure 430. The receiving portion 432 can be centered on a top surface of the closure 430 and have a diameter that is smaller than the closure 430. In some embodiments, the receiving portion 432 can be configured to receive a spout 440. The spout 440 can include an opening 442 that extends through the center of the spout 440. The spout 440 can have a first end 444 and a second end 446. The first end 444 can have a greater diameter than the second end 446. The larger diameter of the first end 444 forms a lip that extends beyond the circumference of the opening 442. As will be discussed in more detail below, the first end 444 of the spout 440 can be engaged such that the spout 440 moves in a proximal direction within the receiving portion 432 of the closure 430 into a first position. In this first position, the opening 442 of the spout 440 can be unsealed to allow the flow of disinfectant/sterilant out of the cartridge 400. In some examples, the first end 444 of the spout 440 can be engaged such that the spout 440 moves in a distal direction within the receiving portion 432 of the closure 430 into a second position. In some embodiments, when in this second position, the opening 442 of the spout 440 can be sealed to prevent the flow of disinfectant/sterilant out of the cartridge 400.

FIGS. 8D-8E illustrate a cross-sectional view of the cartridge 400. FIG. 8E illustrates cross-section A-A which is a lateral cross-section through a center of the cartridge 400. FIG. 8D illustrates an enlarged view of the proximal end of cross-section A-A. FIG. 8D illustrates the spout 440 in the second closed position, while FIG. 8E illustrates spout 440 in the first opened position. The cartridge 400 can include an assembly 490 that secures the components of the cartridge 400 that allow disinfectant/sterilant to be dispensed from the cartridge 400. In some examples, the cartridge 400 includes a filter disk 460 that is circular and secured between an inner top surface of the closure 430 and a top of the opening of the neck 450 of the bottle 410.

In some embodiments, the assembly 490 includes a sump tube adapter 472 and a sump tubing 470. The sump tube adapter 472 can be configured to retain and position the spout 440 and the sump tubing 470 within the cartridge 400. As shown in FIGS. 8D-8E, the spout 440 can include a first end 472a and a second end 472b. The first end 472a can have a greater diameter than the second end 472b. The first end 472a can be configured to receive a securement portion 448 of the spout 440. The securement portion 448 can be located distal to the second end 446 of the spout 440. In some embodiments, the securement portion 448 limits the proximal movement of the spout 440 out of the opening 442 and retains the spout 440 within the closure 430. In some examples, the second end 472b of the sump tube adapter 472 is configured to retain the sump tubing 470. As shown in FIG. 8E, in some embodiments, the sump tubing 470 can extend to the distal-most tip of the bottle 410. The sump tubing 470 can be fluidly connected to the opening 442 of the spout 440. This allow disinfectant/sterilant to be drawn out of the cartridge 400, through the sump tubing 470, and out of the cartridge 400 through the opening 442 of the spout 440. In some examples, the extension of sump tubing 470 to the base of the tapered portion of the bottle 410 maximizes the volume of disinfectant/sterilant in the cartridge 400 dispensed. In some embodiments, the proximal end of the sump tubing 470 is secured within the second end 472b of the assembly 490.

FIGS. 8F-8G illustrate an enlarged cross-sectional view of the cartridge spout. FIG. 8F illustrates an embodiment of the spout 440 of the cartridge 400 when the spout 440 is in a first opened configuration. FIG. 8G illustrates an embodiment of the spout 440 of the cartridge 400 when the spout 440 is in a second closed configuration. As shown in FIGS. 8F-8G, the spout 440 is positioned within the receiving portion 432 of the closure 430. The spout 440 can be lifted out of the receiving portion 432 to allow fluid flow out of the cartridge 400. As discussed above, the spout 440 can include a first end 444 and a second end 446. The first end 444 forms a lip that has a greater diameter than the receiving portion 432 of the closure 430 to limit how far down the spout 440 can move within the receiving portion 432. The second end 446 can include a first portion that has a diameter that can move within the receiving portion 432. The spout 440 can also include a securement portion 448 that extends distally from the second end 446. The securement portion 448 can be configured to engage a lip 431 that extends from an inner surface of the receiving portion 432 to limit the movement of the spout 440 within the receiving portion 432 of the closure 430. In some embodiments, the securement portion 448 includes a primary snap hook 448a and a secondary snap hook 448b. As shown in FIG. 8G, the primary snap hook 448a can engage with the lip 431 to secure the spout 440 within the receiving portion 432 to keep the spout 440 in the second closed configuration to keep the cartridge 400 closed. In some embodiments, primary snap hook 448a of the spout 440 can provide enough retention force to discourage opening the cartridge 400 by any other method than with the cartridge engagement mechanism 300 (discussed above). As shown in FIG. 8F, the primary snap hook 448a can engage with the lip 431 to secure the spout 440 within the receiving portion 432 to position the spout 440 in a first opened position and prevent the spout 440 from extending open too far. In some embodiments, the closure 430 can include a filter disk 460 that is positioned on an underside of the closure 430. The filter disk 460 can serve as a cap vent liner and can be made of an ePTFE material (or other suitable material, including other polymeric materials). The filter disk 460 can allow gases to escape but seal in the liquid. This can be important because hydrogen peroxide decomposes into water and oxygen gas. In some embodiments, the oxygen gas must be vented to prevent buildup of pressure within the cartridge 400.

As discussed above, the spout 440 can include an opening 442 forming a channel that extends through the spout 440. As shown in FIG. 8F, the opening 442 can include a proximal end 442a and a distal end 442b. In some examples, the proximal end 442a has a smaller diameter than the distal end 442b. As shown in FIG. 8F, when the spout 440 is in the second closed position, the opening 442 of the spout 440 can be sealed on the conical seat 436. The conical seat 436 of the closure 430 can include a proximal end 436a and a distal end 436b. When the spout 440 is in the second closed position, the proximal end 436a is secured within the proximal end 442a of the opening 442 and the distal end 436b is secured within the distal end 442b to prevent fluid flow out of the closure 430 of the cartridge 400. As will be discussed in more detail below, the proximal end 436a of the conical seat 436 has a diameter that is less than the internal diameter of the distal end 436b of the conical seat 436. This can allow fluid to flow through the distal end 436b and around the proximal end 436a out of the opening 442 when the spout 440 is in a first opened position.

In some embodiments, the internal surface of the receiving portion 432 can include a plurality of sealing ribs 433 to engage with an outer surface of the second end 446 of the spout 440. The outer surface of the distal end 436b can include a plurality of sealing ribs 435 to engage an inner surface of the distal end 442b. The sealing ribs 433 and the sealing ribs 435 can allow the closure 430 and the spout 440 to provide sealing surfaces.

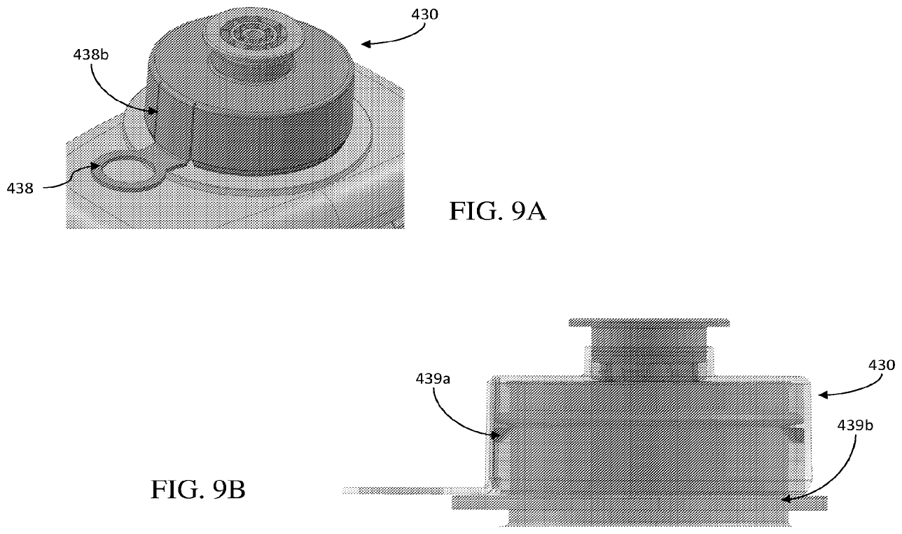

The closure 430 of the cartridge 400 can be configured to allow a user to remove the closure 430 from the bottle 410. As illustrated in FIG. 9A, the closure 430 can include a pull ring 438 and a plurality of thin areas 438a. The thin areas 438a can tear when the pull ring 438 is pulled or twisted with sufficient force. As shown in FIG. 9B, the closure 430 can include a plurality of engagement hooks 439a that secure the closure 430 to the bottle 410. When the pull ring 438 is pulled, the pull ring 438 can also release one of two of the engagement hooks 439a. In some embodiments, the closure 430 comprises a material such as HDPE or LDPE. Once the pull ring 438 is pulled, the closure 430 can be removed from the cartridge 400. To properly dispose of the cartridge 400, the cartridge 400 may need to be rinsed out to dilute any remaining peroxide residue in the bottle 410 prior to disposal.

In some embodiments, the pulling of the pull ring 438 and removing of the closure 430 can render the cartridge 400 unable to be reused in the disclosed system. In some embodiments, the engagement hooks 439a engage at a steep angle to the bottle 410 to prevent the closure 430 to be pried off the bottle 410 and increase the tamper-resistance of the cartridge 400. This can prevent improper refilling of the bottle 410. In some embodiments, the closure 430 can include a weak edge 439b that is positioned around the base of the closure 430. In some embodiments, the base of the closure 430 can be close enough to the bottle 410 to prevent the insertion of tools used to pry the closure 430 off the bottle 410 of the cartridge 400. In some examples, the weak edge 439b can deflect and deform easily if a prying tool is inserted, which can reduce the force a prying tool may apply on lifting the closure 430 from the bottle 410.

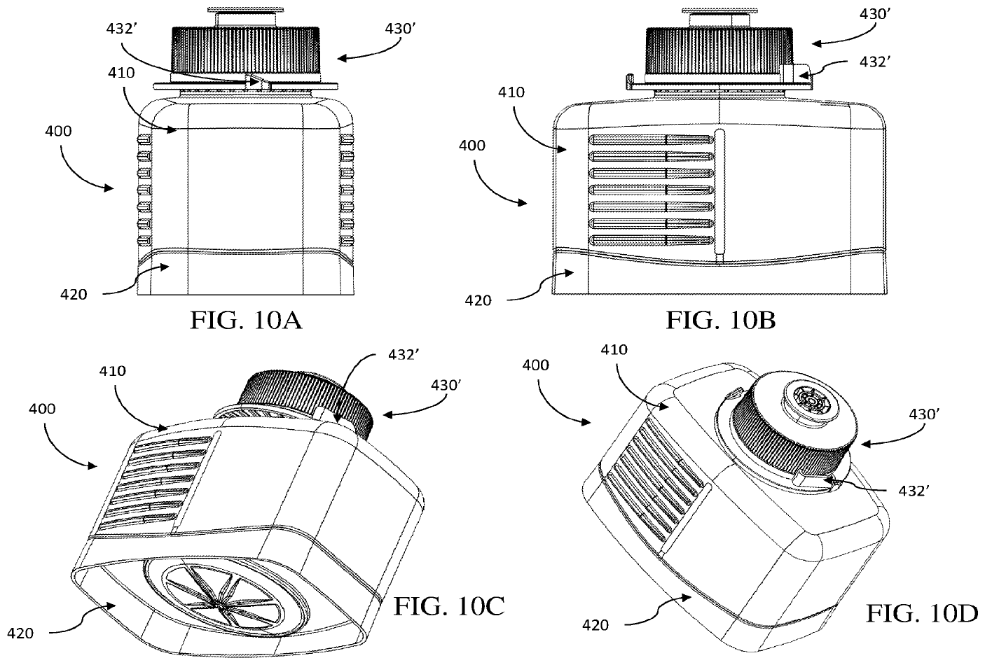

FIGS. 10A-10D illustrates another embodiment of the closure 430′ of the cartridge 400. In some embodiments, the closure 430′ includes a receiving portion 432′ such that the closure 430′ can only be removed using multiple actions. In some embodiments, the receiving portion 432′ must first be depressed before the closure 430′ can be unscrewed. This can provide an additional securement mechanism to prevent the closure 430′ of the cartridge 400 from unintentionally opened.

### Device Fluid Flow

### Housing Fluid Flow

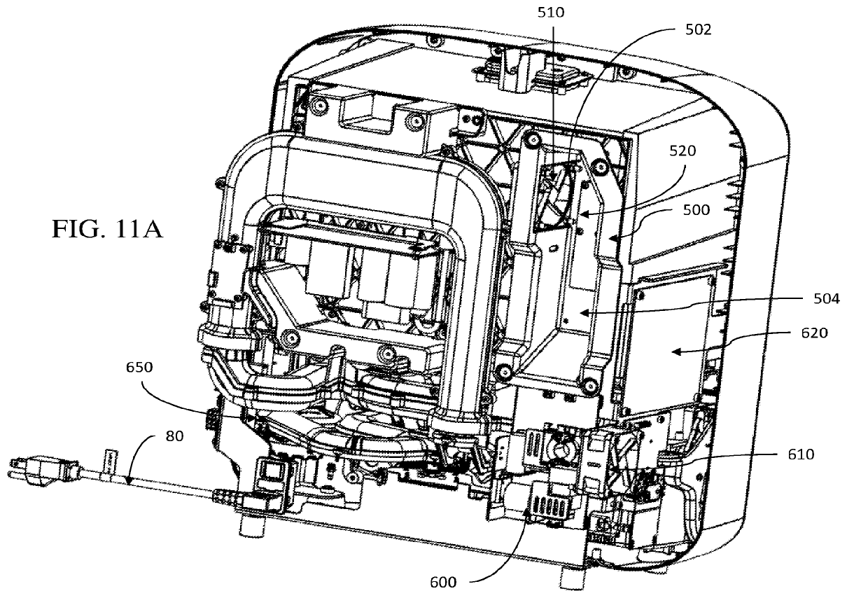

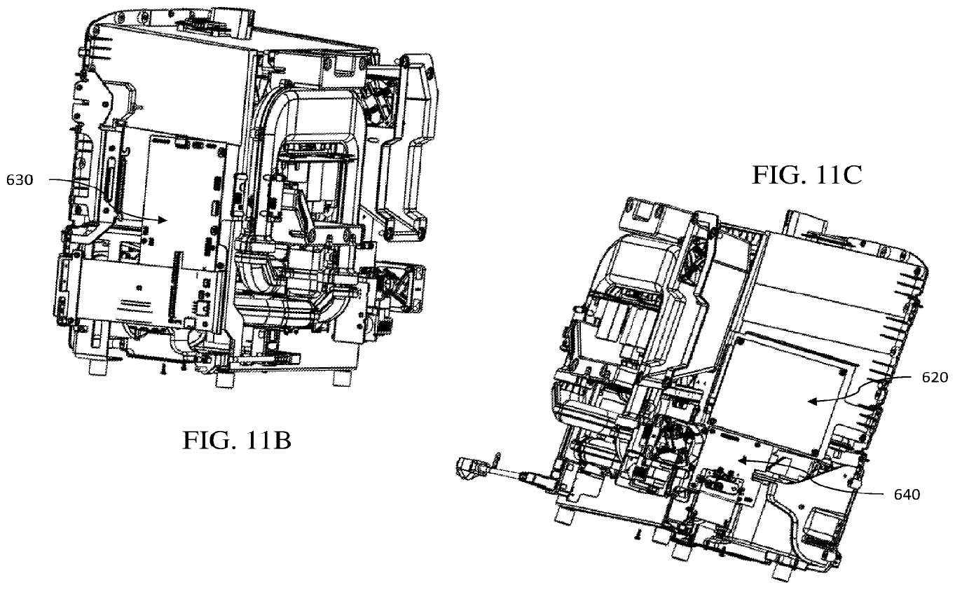

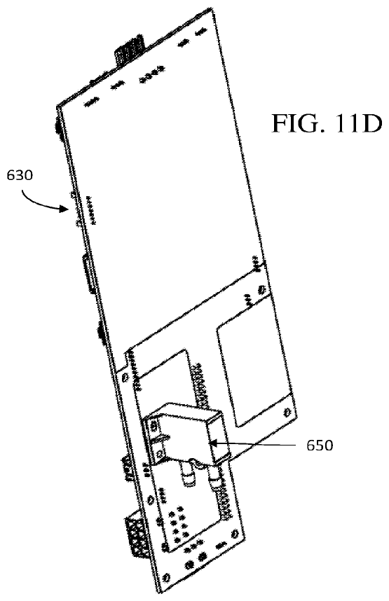

FIGS. 11A-11C illustrates a plurality of rear perspective views of the device 10 with the rear panel 24 removed. As shown, the housing 20 can include a fan baffle 500 with an opening 502 that secures a fan 510. In some embodiments, an ambient ozone sensor 520 is positioned on the interior 504 within the fan baffle 500. As shown in FIG. 11C, the fan baffle 500 is positioned adjacent to the vent 64 of the exhaust 60 on the rear panel 24. As will be discussed in more detail below, once the disinfectant and/or sterilant is exhausted from the system through the exhaust filter 240, the airflow is blown into the interior of the housing 20. In some embodiments, the fan 510 draws the airflow within the housing through the opening 502, into the fan baffle 500, and out of the exhaust 60 of the rear panel 24. The fan 510 can move air flow past the ambient ozone sensor 520 and out of the housing 20. The movement of the fluid flow 90 is illustrated in FIG. 11C. In some embodiments, the fan baffle 500 is configured to limit noise exiting the housing 20. In some examples, the ambient ozone sensor 520 is positioned adjacent to the vent 64 of the rear panel 24 such that all airflow leaving the housing 20 contacts the ambient ozone sensor 520. This can be done with or without the fan baffle 500.

As mentioned above, the airflow with the housing 20 is collected into the fan baffle 500. If the ambient ozone sensor 520 detects that levels are higher than the preset threshold, the ambient ozone sensor 520 will detect a system failure and shut everything down. In some embodiments, the ambient ozone sensor 520 is sensitive and able to detect ozone in much lower parts per million. In some examples, the threshold detected by the ambient ozone sensor 520 is much lower than the level detected in the system pathway. In some embodiments, the threshold of ozone detected by the ambient ozone sensor 520 is less than 0.10 PPM, less than 0.09 PPM, less than 0.08 PPM, less than 0.07 PPM, less than 0.06 PPM, less than 0.05 PPM, less than 0.04 PPM, less than 0.03 PPM, less than 0.02 PPM, less than 0.01 PPM, between 0.09 PPM-0.10 PPM, between 0.08 PPM-0.09 PPM, between 0.07 PPM-0.08 PPM, between 0.06 PPM-0.07 PPM, between 0.05 PPM-0.06 PPM, between 0.04 PPM-0.05 PPM, between 0.03 PPM-0.04 PPM, between 0.02 PPM-0.03 PPM, between 0.01 PPM-0.02 PPM, and any value in between those ranges listed, including endpoints. In some embodiments, the ambient ozone sensor 520 is configured to ensure operator safety based on OSHA standards. In some examples, the ambient ozone sensor 520 is also configured to monitor any leaks and dysfunctions in the system of the device 10.

In some embodiments, when the airflow is exhausted from the exhaust filter 240, the airflow is diluted for a preset time and mixes the moist air with additional air drawn into the system and housing 20 of the device 10 through the inlet filter 230. In some examples, the air is exhausted for less than 1 minute, less than 2 minutes, less than 3 minutes, less than 4 minutes, less than 5 minutes, between 0-1 minutes, between 1-2 minutes, between 2-3 minutes, between 3-4 minutes, between 4-5 minutes, and any value in between those ranges listed, including endpoints. In some embodiments, there can be approximately 2 mL of moisture in the air flow flowing out of the system of the device 10. In some examples, this moisture is blended with air drawn in from the inlet filter 230 for the preset time. This can prevent condensation (e.g., humidity) from forming on the surface of the circuitry as the airflow moves through the housing 20 of the device 10 and out of the vent 64 in the rear panel 24.

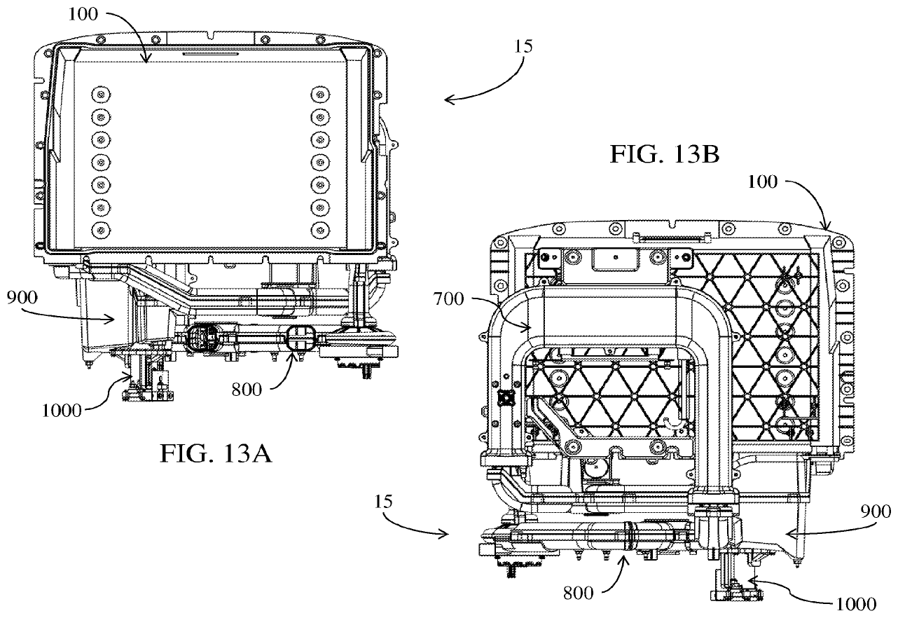

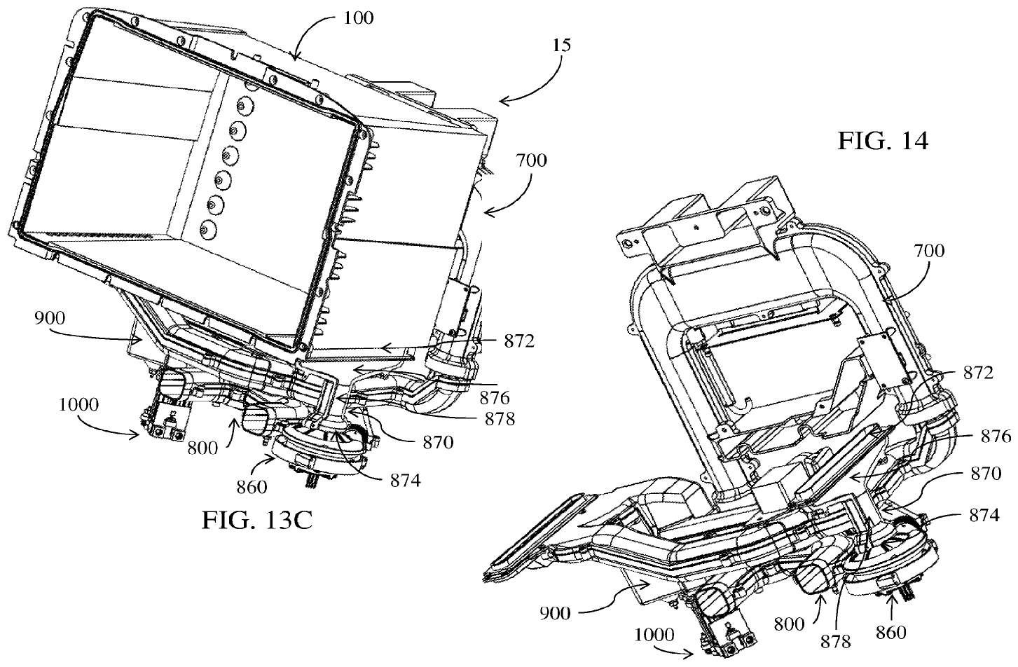

In some embodiments, the system 15 operates at ambient temperature. In some examples, the airflow through the chamber 100 is maintained at an ambient temperature. In some examples, the chamber 100 can include a heating component to allow heat distribution to slightly elevate the temperature of the walls. In some embodiments, this will only slightly increase the interior of the device to approximately 100 degrees F. Fahrenheit (F), 102 degrees F., 104 degrees F., 106 degrees F., 108 degrees F., 110 degrees F., 112 degrees F., 114 degrees F., 116 degrees F., 118 degrees F., 120 degrees F., 122 degrees F., 124 degrees F., 126 degrees F., 128 degrees F., 130 degrees F., between 100-102 degrees F., between 102-104 degrees F., between 104-106 degrees F., between 106-108 degrees F., between 108-110 degrees F., between 110-112 degrees F., between 112-114 degrees F., between 114-116 degrees F., between 116-118 degrees F., between 118-120 degrees F., between 120-122 degrees F., between 122-124 degrees F., between 124-126 degrees F., between 126-128 degrees F., between 128-130 degrees F., and any value in between those ranges listed, including endpoints. In some examples, the additional heating elements can be configured to control the humidity and condensation inside the airflow of the system. The airflow of the system 15 will be described in more detail below in FIGS. 13A-13C and 14.

In some embodiments, the system 15 operates at or above a predetermined startup temperature, or equilibration temperature. In some embodiments, the predetermined startup temperature is a temperature wherein one or more components, including the chamber, are equilibrated within a set temperature range. In some examples, the airflow through the chamber 100 is maintained at the predetermined startup temperature. In some examples, the chamber 100 can include a heating component to allow heat distribution to elevate the temperature of the walls. In practice, elevating the temperature of the walls allows for enhanced reproducibility and equilibration of the entire system between runs. In some embodiments, the predetermined startup temperature will increase the interior of the device to approximately about between 40 degrees centigrade and 50 degrees centigrade (about between 104 degrees F. to about between 122 degrees F.). In some embodiments, the predetermined startup temperature will increase the interior of the device to approximately about between 25 degrees centigrade and 55 degrees centigrade, or any value in between. In some embodiments, the walls of the device have an equilibrated temperature of approximately 100 degrees F. Fahrenheit (F), 102 degrees F., 104 degrees F., 106 degrees F., 108 degrees F., 110 degrees F., 112 degrees F., 114 degrees F., 116 degrees F., 118 degrees F., 120 degrees F., 122 degrees F., 124 degrees F., 126 degrees F., 128 degrees F., 130 degrees F., between 100-102 degrees F., between 102-104 degrees F., between 104-106 degrees F., between 106-108 degrees F., between 108-110 degrees F., between 110-112 degrees F., between 112-114 degrees F., between 114-116 degrees F., between 116-118 degrees F., between 118-120 degrees F., between 120-122 degrees F., between 122-124 degrees F., between 124-126 degrees F., between 126-128 degrees F., between 128-130 degrees F., and any value in between those ranges listed, including endpoints. In some examples, the additional heating elements can be configured to control the humidity and condensation inside the airflow of the system. In some embodiments, components of the system, including the chamber 100, can comprise a material suitable for quickly increasing or decreasing heat, such as glass, ceramic, plastic, or metal and metal covered with Teflon or other protective layer. In some embodiments, one or more heating elements can be located at one or more locations throughout the system. In some embodiments, the system further comprises insulation or insulating materials as outer cladding, in order to specifically modulate and direct heat flow to optimize for example, temperature equilibration or temperature based purge cycles (i.e. temperature conditioning steps).

### Additional Components and Sensors

In some embodiments, the device 10 includes an ozone power supply PCB 620. In some embodiments, the ozone power supply PCB 620 can take wall voltage (e.g., 24V) and convert it to a high voltage power supply (e.g., 170V DC). In some examples, the high voltage power supply (e.g., 170V DC) is a high enough voltage for the ozone generator 740 (discussed in more detail below) to use to make several kilovolts and also to regulate the voltage supplied to the ozone generator 740. This can be important as ozone generation is dependent on supply voltage.

In some examples, the device 10 includes a power supply 600 with a power supply fan 610. In some embodiments, the power supply 600 can convert municipal wall power of either 120V or 220V and either 50 hz AC or 60 hz AC and converts it to 24V for use by the components within the system 15.

In some embodiments, the device 10 includes a main driver PCB 630. In some embodiments, the blower 860 can include its own power modulator. In some embodiments, the main driver PCB 630 includes a leak rate sensor 650. In some embodiments, the leak rate sensor 650 detects how much air is being driven out of the machine. In some examples, this is roughly equivalent to the amount of air coming into the machine from leakage. As shown in FIG. 11D, the leak rate sensor 650 can include a plurality of barbed fittings (e.g., two barbed fittings) through which air leakage can travel. In some embodiments, expansion of the air flow from temperature increase and the evaporation of the liquid disinfectant and/or sterilant (e.g., peroxide and water) can also flow through the leak rate sensor 650.

In some examples, the device 10 includes a piezo PCA 640. In some embodiments, the piezo PCA is configured to control a plurality of pumps fluidly connected to the nebulizer 1000. In some embodiments, the piezo PCA 640 can control the piezocrystal 1060 in the nebulizer 1000. In some examples, the plurality of pumps fluidly connected to the nebulizer 1000 can be any precision pump. In some embodiments, the plurality of pumps fluidly connected to the nebulizer 100 can be any piezo based pump. In some embodiments, the sterilant is delivered by one or more piezo pumps.

### Overview Fluid Flow

FIGS. 13A-13C illustrates an embodiment of the system for disinfection and/or sterilization 15 for disinfecting and/or sterilizing an item placed within the chamber 100. As discussed above, airflow travels through the system 15 and carries disinfectant/sterilant (e.g., ozone, peroxide) to the chamber 100 before it exits the system 15 through the outlet filter 240 and into the housing 20. FIG. 14 illustrates the system 15 with the chamber 100 removed. FIG. 14 is included to provide a better visualization of the interconnectivity of the system 15.

As will be discussed in more detail below, the system 15 can include a chamber 100, an ozone generation system 700, an evaporator 900, and a nebulizer 1000. In some embodiments, the system 15 can include ducting 800 that is configured to control whether air flows through the inlet filter 230 and/or the outlet filter 240.

### Ozone Generation System

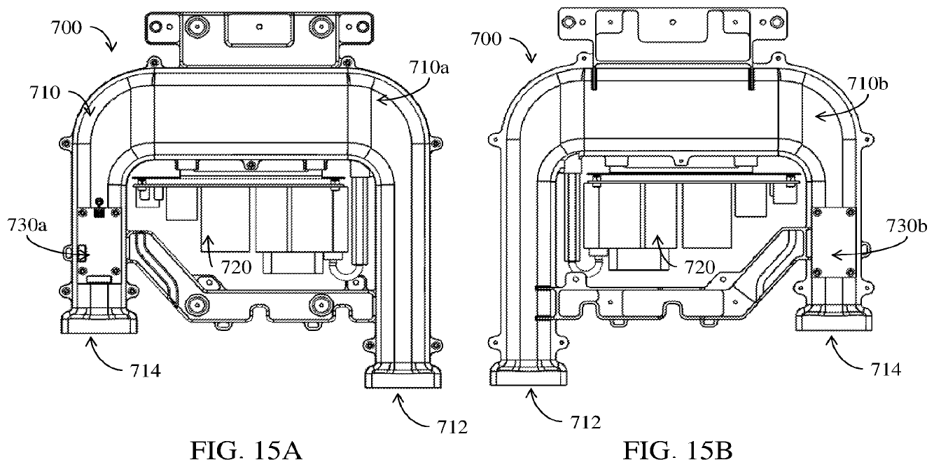

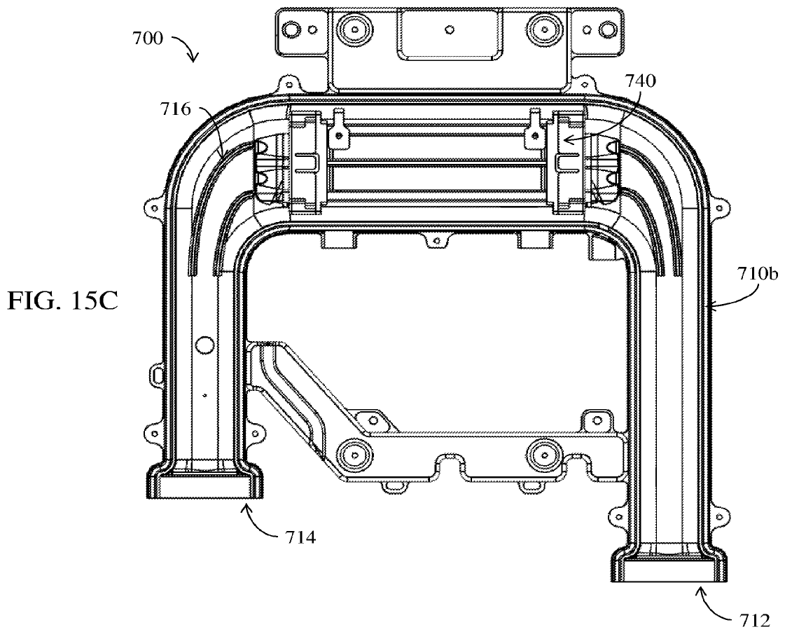

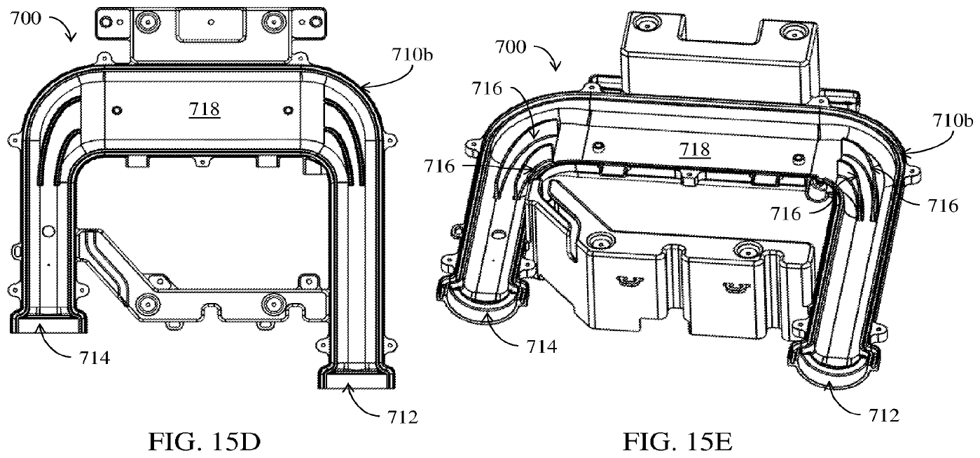

FIGS. 15A-15B illustrates an embodiment of an ozone generation system 700 in the device 10. FIG. 15C illustrates the ozone generation system 700 with the ozone duct cap 710a of the ozone duct 710 removed to show the ozone generator 740 positioned in the ozone duct 710.

As shown, the ozone generation system 700 includes the ozone duct 710 that includes an ozone duct cap 710a and an ozone duct base 710b. The ozone generation system 700 includes the ozone generator 740 positioned between the ozone duct cap 710a and the ozone duct base 710b. The ozone duct 710 includes an inlet 712 and an outlet 714. In some embodiments, the inlet 712 of the ozone duct 710 receives airflow from the outlet of the ducting 800. In some examples, the outlet 714 directs airflow to the inlet of the evaporator 900. In some embodiments, the ozone duct cap 710a and the ozone duct base 710b includes a plurality of fins 716. In some examples, the fins 716 can serve as guide vanes to reduce a pressure drop of a fluid flow through a bend of the ozone duct 710. In some examples, the bends and curves of the ozone duct 710 of the ozone generation system 700 are large and smooth in order to reduce airflow restrictions. This can help to reduce the blower power required which, in turn, reduces noise.

In some embodiments, the ozone generation system 700 can include a power supply 720 to the ozone generator 740. As will be discussed in more detail below, the power supply 720 can be configured to ensure that the ozone generator 740 receives a constant supply of voltage.

In some examples, the ozone generation system 700 can include an ozone sensor 730. As shown in FIGS. 15A-15B, a first portion of the ozone sensor 730a can be positioned on the ozone duct cap 710a and a second portion of the ozone sensor 730b is positioned on the ozone duct base 710b. In some embodiments, the ozone sensor 730 is configured to detect the amount of ozone in the airflow through the ozone generation system 700 and is configured to adjust the duty cycle of the ozone generator 740 to keep the concentration of ozone in the airflow within a narrow range. In some examples, the ozone sensor 730 can be configured to determine the density of the air in order to know the proper concentration of ppm of ozone. This can allow the device to operate in various geographies where the density of air may vary.

FIGS. 15D-15E illustrates the ozone duct base 710b of the ozone generation system 700 with the ozone generator 740 removed. As shown, the ozone generator 740 can be positioned on the body 718 of the ozone generation system 700 between the bends of the ozone duct 710. In some embodiments, the ozone generator 740 is positioned between the fins 716 such that airflow is guided along parallel paths through the ozone generator 740.

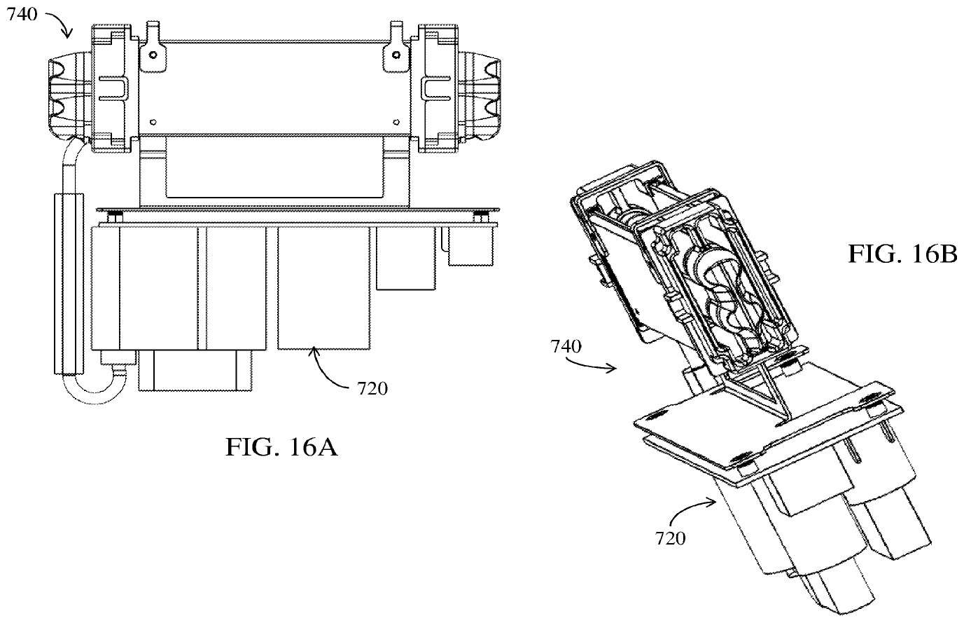

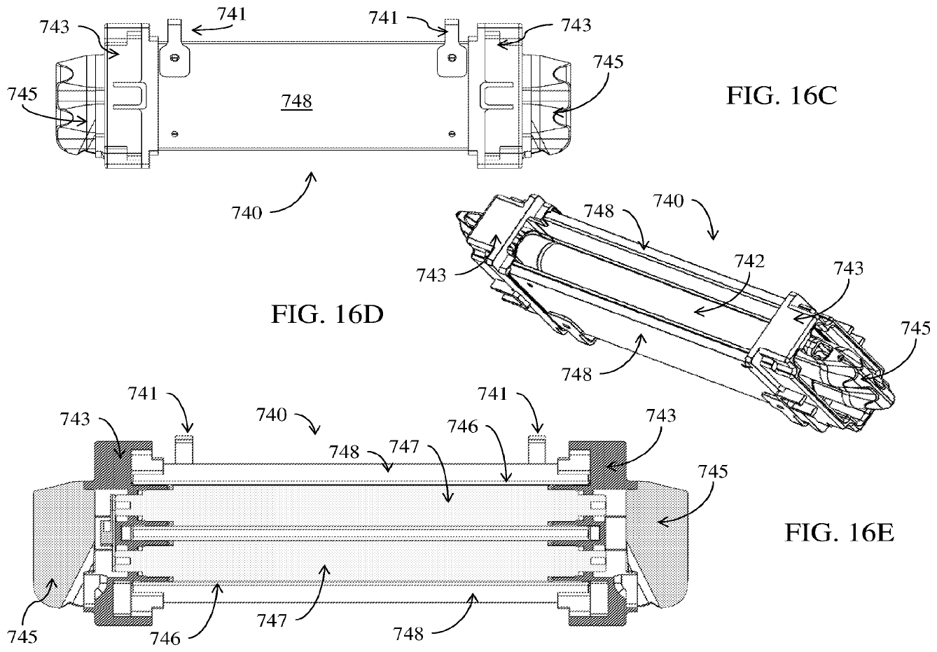

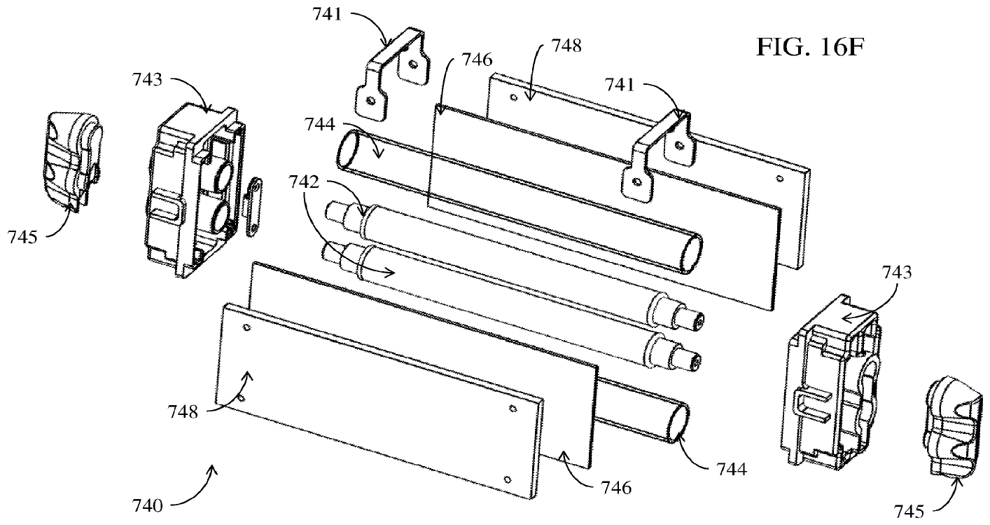

FIGS. 16A-16B illustrates an embodiment of the ozone generator 740 attached to the power supply 720. As ozone generation can be dependent on voltage, the power supply 720 is configured to ensure that input voltage is constant and regulated regardless of the power provided to the device 10. In some embodiments, the power supply 720 is configured to convert any power supply (e.g., rear panel 24 V DC supply) and step it up to outlet openings 170 V. This ensures that, if the power provided becomes inconsistent, the system will generate an error code to indicate that the ozone levels required cannot be generated given the power supply provided.

FIGS. 16C-16D illustrate front and a top perspective views of the ozone generator 740. FIG. 16E illustrate a cross-sectional view of the ozone generator 740 and FIG. 16F provides an exploded view of the ozone generator 740 to show the components of the ozone generator 740. In some embodiments, the ozone generator 740 includes a pair of bars 747. In some examples, each of the bars 747 includes an electrode centers 742 with a glass tube 744 disposed over the electrode center 742. In some embodiments, the electrode center 742 comprises aluminum. In some examples, the pair of bars 747 are secured on either side with a butt cap 743 and a cap electrode 745. In some embodiments, the pair of bars 747 are secured between a pair of dielectric ground plates 746. In some examples, the pair of dielectric ground plates 746 are secured between a pair of electrode ground plates 748 that are secured through a pair of ground jumpers 741.

In some embodiments, the ozone generator 740 provides for an electrical discharge between the pair of bars 747 with the adjacent pair of dielectric ground plates 746. In some examples the duty cycle is preset. For examples, the duty cycle can be approximately 50%. In some embodiments the ozone generator 740 calculates a percentage of the next three (3) seconds the ozone generator 740 will be on for and turns on for that percentage of it. In some examples, the ozone generator 740 can be turned on for 100% or 0% of the time. This can provide pulse-width modulating. In some embodiments, the duty cycle can be 10%, 15%, 20%, 25%, 30%, 35%, 40%, 45%, 50%, 55%, 60%, 65%, 70%, 75%, 80%, 85%, 90%, 95%, 100%, between 10-15%, between 15-20%, between 20-25%, between 25-30%, between 30-35%, between 35-40%, between 40-45%, between 45-50%, between 50-55%, between 55-60%, between 60-65%, between 65-70%, between 70-75%, between 75-80%, between 80-85%, between 85-90%, between 90-95%, between 95-100%, and any value in between those ranges listed, including endpoints. In some embodiments, the operating frequencies can be very high. The operating frequency can be faster than 1/30 seconds or 1/1 minute to prevent large ozone concentration variations. In some examples, the ozone generator 740 is on for a variable portion of the preset time. This can change depending on the information provided by the ozone sensor 730 that indicates whether sufficient ozone is being provided to the airflow. If ozone concentration is insufficient, the ozone generator 740 will be turned on for a longer period of time (e.g., higher duty cycle); if ozone concentration is sufficient, the ozone generator 740 will be turned on for a shorter period of time. In some embodiments, the duty cycle is more than 50% (e.g., three (3) seconds) to allow for the build-up and subsequent discharge of the ozone generator 740. In some examples, the ozone generator 740 is controlled by the input from a UV LED sensor.

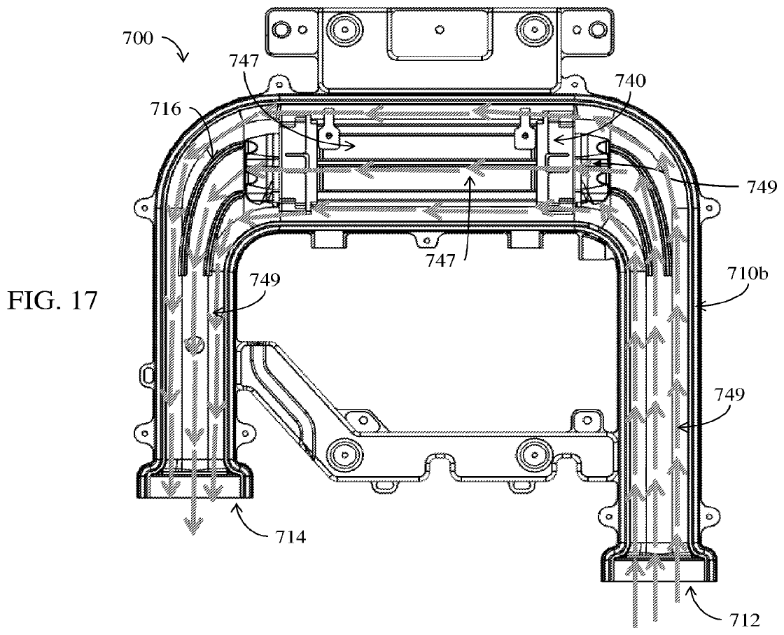

FIG. 17 illustrates a schematic of the airflow through the ozone duct 710 of the ozone generation system 700 and past the ozone generator 740. Fluid flow 749 through the ozone duct 710 is shown by the arrows flowing from the inlet 712 of the ozone duct 710 to the outlet 714 of the ozone duct 710. As shown in FIG. 17, the airflow can flow parallel to the bars 747 and across much of the general area. This can help to improve the efficiency of the ozone generator 740. As ozone reacts with itself to reform oxygen, the design illustrated in ozone generator 740 limits the destruction of ozone by ensuring constant airflow and by increasing the amount of air moving past the ozone generator 740.

### Blower Ducting

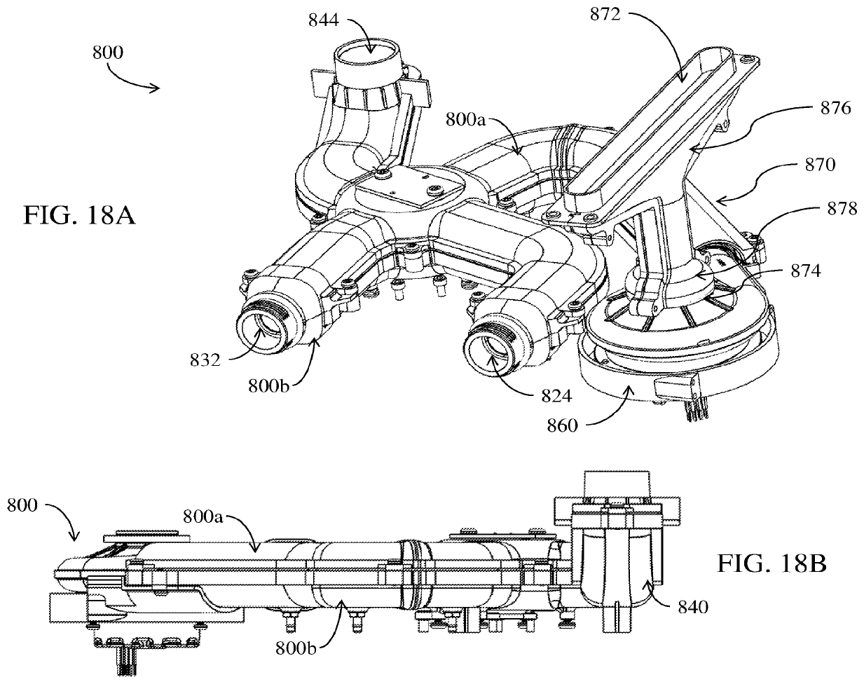

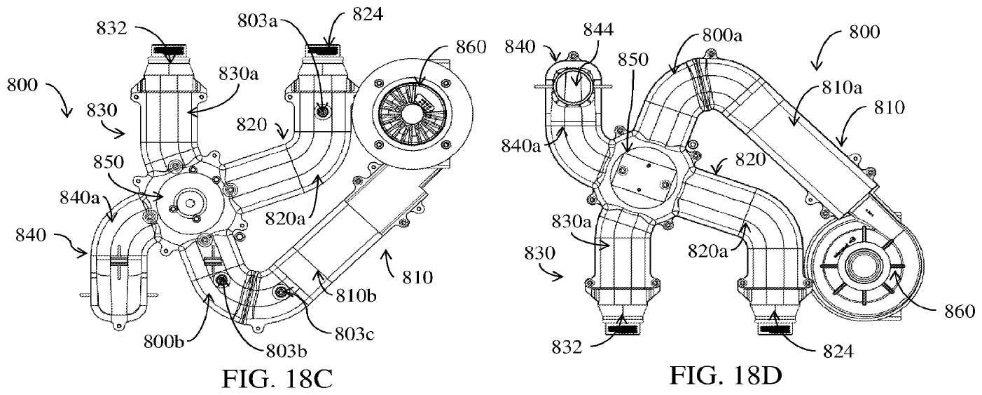

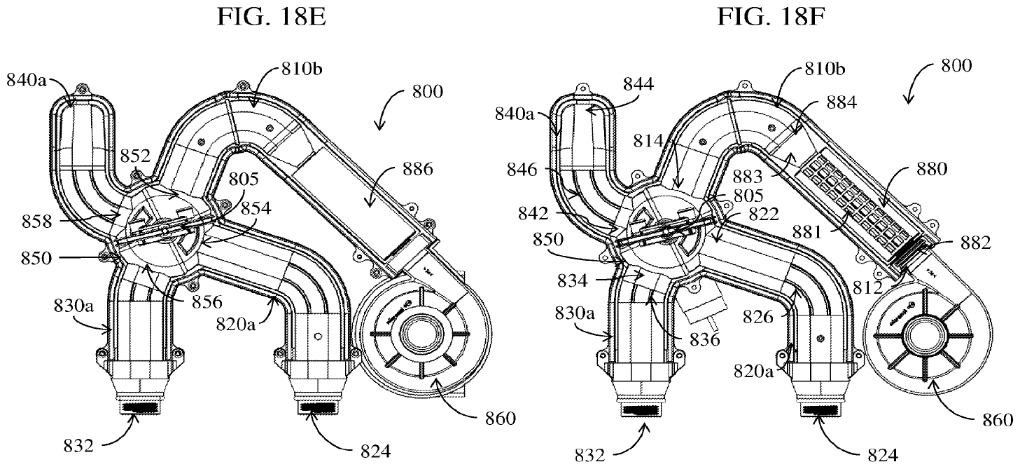

FIGS. 18A-18B provides a perspective and a side view of the ducting 800. FIGS. 18C-18D illustrates a top view of the ducting 800 and a bottom view of the ducting 800. FIGS. 18E and 18F illustrates a top view of the ducting 800 with the top portion 810a removed to show the muffler 880 with and without the muffler foam 886.

In some embodiments, the ducting 800 is configured to receive fluid flow from the blower 860. The ducting 800 can received fluid flow from the chamber 100 through the adaptor 870. In some embodiments, the adaptor 870 includes a first portion 876 and a second portion 878 that extends from an inlet 872 to an outlet 874. As shown in FIG. 18A, the first portion 876 is tapered and reduces in diameter from a wider inlet 872 to a narrower outlet 874. In some embodiments, the diameter of the inlet 872 is greater than the diameter of the outlet 874. As will be discussed in more detail below, in some examples, the length of the inlet 872 corresponds with the plurality of outlet openings 170 that receives fluid flow exiting the chamber 100. In some examples, airflow moves out of the outlet 874 of the adaptor 870 and into the blower 860. In some embodiments, the airflow can move out of the blower 860 and into the inlet 812 of the ducting 800.

FIGS. 18C-18D illustrates the ducting 800 that includes a top portion 800a and a bottom portion 800b. In some embodiments, the ducting 800 includes a first portion 810, a second portion 820, a third portion 830, and a fourth portion 840. In some examples, the first portion 810, the second portion 820, the third portion 830, and the fourth portion 840 are fluidly connected at the central hub 850. As shown in FIG. 18E, the central hub 850 can include an opening 852, an opening 854, and opening 856, and an opening 858. In some embodiments, the central hub 850 is configured to secure a valve 805 such that the valve 805 can rotate between a first position and a second position.

In some embodiments, the first portion 810 can include a top portion 810a and a bottom portion 810b. The first portion 810 can extend between an inlet 812 and an outlet 814. In some embodiments, the first portion 810 is configured to direct airflow from the blower 860 and into the opening 852 of the central hub 850.

In some examples, the second portion 820 can include a top portion 820a and a bottom portion 820b. The second portion 820 can extend between an inlet 822 and an outlet 824. In some embodiments, the second portion 820 is configured to direct airflow out of the opening 854 of the central hub 850 and out of the outlet 824. In some examples, airflow moves out of the outlet 824, through the inlet 242 of the filter 240 and out of the outlet 244 of the filter 240.

In some embodiments, the third portion 830 can include a top portion 830a and a bottom portion 830b. The third portion 830 can extend between the inlet 832 and the outlet 834. In some embodiments, the third portion 830 is configured to direct airflow from the inlet filter 230 into the inlet 832 and into the opening 856 of the central hub 850. In some examples, airflow is sucked through the inlet 232 of the filter 230, through the outlet 234 of the filter 230, and into the opening 856 of the central hub 850.

In some examples, the fourth portion 840 can include a top portion 840a and a bottom portion 840b. The fourth portion 840 can extend between the inlet 842 and the outlet 844. In some embodiments, the outlet 844 is positioned in the top portion 840a. In some examples, the fourth portion 840 is configured to direct airflow from the opening 858 of the central hub 850, through the inlet 842, and out of the outlet 844 of the fourth portion 840. In some embodiments, the fourth portion 840 is configured to direct airflow out of the ducting 800 and into the inlet 712 of the ozone generation system 700.

In some embodiments, the ducting 800 can include a plurality of fittings 803a, 803b 803c along the path of the ducting 800. In some examples, any of the plurality of fittings 803a, 803b 803c can be fluidly connected to the prongs of the leak rate sensor 650. The ducting 800 can include large and smooth airflow passages (e.g., along the bends) that are configured to reduce the noise the system makes. In some embodiments, the large widths of the ducting 800 can provide for low restriction. In some examples, the ducting 800 can have a duct flow area greater than 100 mm², greater than 200 mm², greater than 300 mm², greater than 400 mm², greater than 500 mm², greater than 600 mm², greater than 700 mm², greater than 800 mm², greater than 900 mm², greater than 1000 mm², greater than 1100 mm², greater than 1200 mm², greater than 1300 mm², greater than 1400 mm², less than 1500 mm², between 1400-1500 mm², between 1300-1400 mm², between 1200-1300 mm², between 1100-1200 mm², between 1000-1100 mm², between 900-1000 mm², between 800-900 mm², between 700-800 mm², between 600-700 mm², between 500-600 mm², between 400-500 mm², between 300-400 mm², between 200-00 mm², between 100-200 mm², and any value in between those ranges listed, including endpoints. Low restriction along the length of the ducting 800 ensures that there is a low pressure drop along the length of the ducting 800. In some examples, a low pressure drop requires low blower power which creates lower noise.

As mentioned above, FIGS. 18E-18F provide a view of the ducting 800 with the top portion 800a removed. In some embodiments, each of the first portion 810, second portion 820, third portion 830, and fourth portion 840 includes a plurality of fins. As shown, the second portion 820 can include a plurality fins 826, the third portion 830 can include a plurality of fins 836, and the fourth portion 840 can include a plurality of fins 846. In some examples, the plurality of fins 826, 836, 846, can serve as guide vanes to reduce a pressure drop of fluid flow through the bends of the associated lengths of the ducting 800.

In some embodiments, the ducting 800 can include a muffler 880 to reduce the noise generated by the blower 860. In some embodiments, as shown in FIG. 18E, the muffler 880 can include muffler foam 886. In some examples, the blower 860 comprises a fibrous insulation material. In some examples, the muffler 880 can include a first portion 881 and a second portion 883. The muffler 880 can extend between the inlet 882 at a first end of the first portion 881 and the outlet 884 at a second end of the second portion 883. In some embodiments, the first portion 881 can include a plurality of openings to reduce the noise of airflow moving through the ducting 800. In some embodiments, the diameter of the second portion 883 increases from a first end to the outlet 884 of the muffler 880. In some examples, the muffler 880 can be positioned in the bottom portion 810b of the first portion 810 adjacent to the blower 860. In some embodiments, the inlet 882 of the muffler 880 and the inlet 812 of the first portion 810 are configured to engage with an outlet of the blower 860.

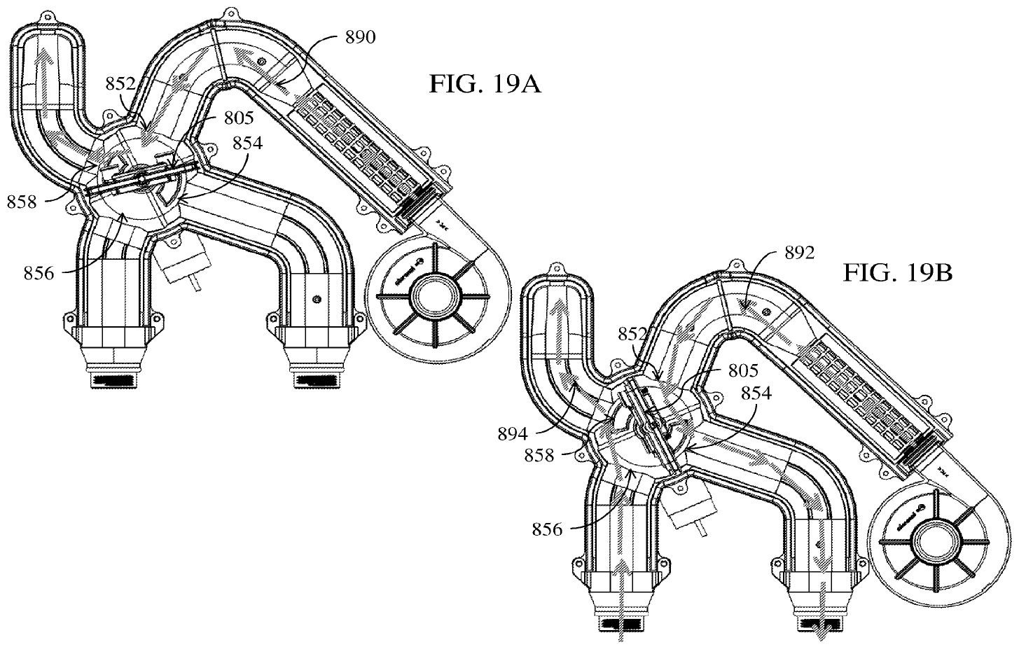

FIGS. 19A-19B illustrates a top view of the ducting 800 with a schematic of the fluid flow through the ducting 800 when the valve 805 is in a first position (e.g., FIG. 19A) and when the valve 805 is positioned in a second position (e.g., FIG. 19B). In some embodiments, the valve 805 is configured to rotate approximately 90 degrees between the first position and the second position.

As shown, in FIG. 19A, the valve 805 is positioned between the opening 852 and the opening 854 at a first end and between the opening 856 and the opening 858 at a second end. When in this first position, the valve 805 can block off the pathways to the filters 230, 240 (i.e., airflow to second portion 820 and third portion 830). The movement of the fluid flow 890 is indicated in arrows in FIG. 19A

As shown in FIG. 19B, the valve 805 is positioned between the opening 852 and the opening 858 at a first end and between the opening 854 and the opening 856 at a second end. In some embodiments, when in this second position, the valve 805 is configured to allow airflow from the blower 860 to the outlet filter 240 (i.e., from the first portion 810 to the second portion 820). The movement of the fluid flow 892 is indicated in arrows in FIG. 19B. In some examples, the second position also allows the valve 805 to allow airflow from the inlet filter 230 to the inlet 712 of the ozone generation system 700 (i.e., from the third portion 830 to the fourth portion 840). The movement of the fluid flow 894 is indicated in arrows in FIG. 19B.

### Evaporator and Nebulizer

### Overview

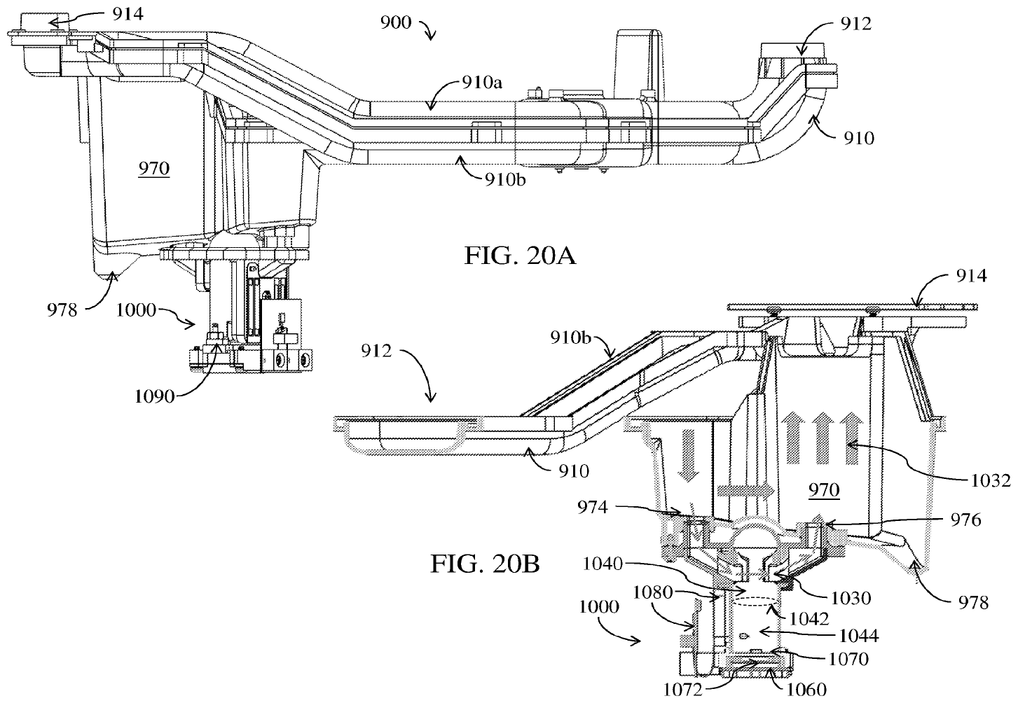

FIGS. 20A-20B illustrates an embodiment of an evaporator 900 fluidly connected with a nebulizer 1000. FIG. 20A illustrates a front view of the evaporator 900 and the nebulizer 1000. In some examples, the nebulizer 1000 is configured to create the disinfectant/sterilant (e.g., hydrogen peroxide) into a mist. The evaporator 900 can then transfer the disinfectant/sterilant mist into the airflow where it is delivered to the chamber 100.

In some embodiments, the evaporator 900 includes a ducting 910. In some examples, the ducting 910 includes a cap 910a and a base 910b. The ducting 910 can receive airflow leaving the outlet 714 of the ozone generation system 700 at the inlet 912 of the evaporator 900. In some examples, the ducting 910 can conduct airflow to the outlet 914 where air, with or without disinfectant/sterilant is moved into the chamber 100 through the inlet openings 160. FIG. 20B illustrates a cross-sectional view of the evaporator 900 fluidly connected with the nebulizer 1000. As will be discussed in more detail below, airflow can be blown through the ducting 910 of the evaporator 900 to either flow through the nebulizer 1000 or to bypass the nebulizer 1000 entirely.

### Evaporator

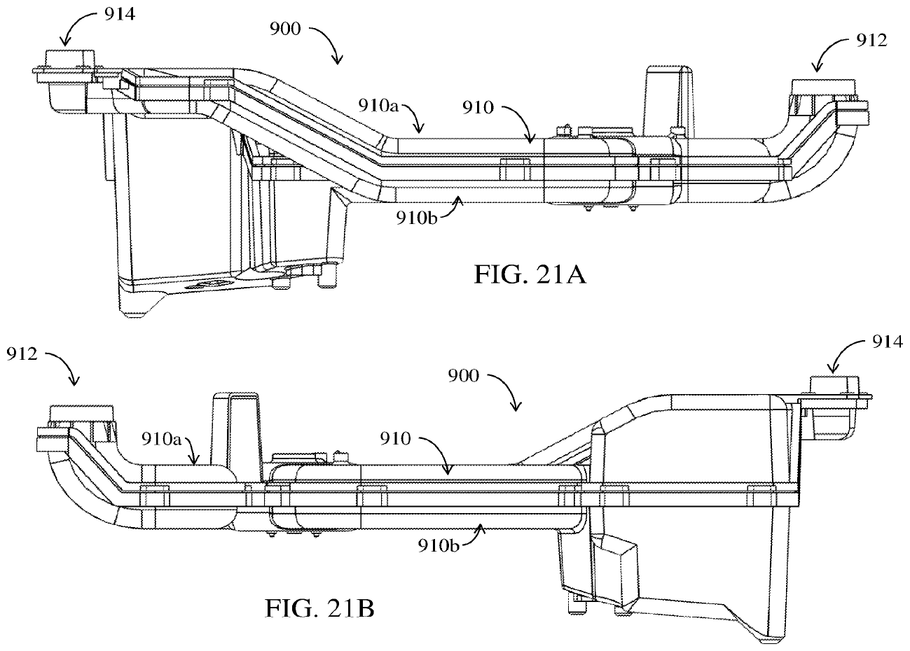

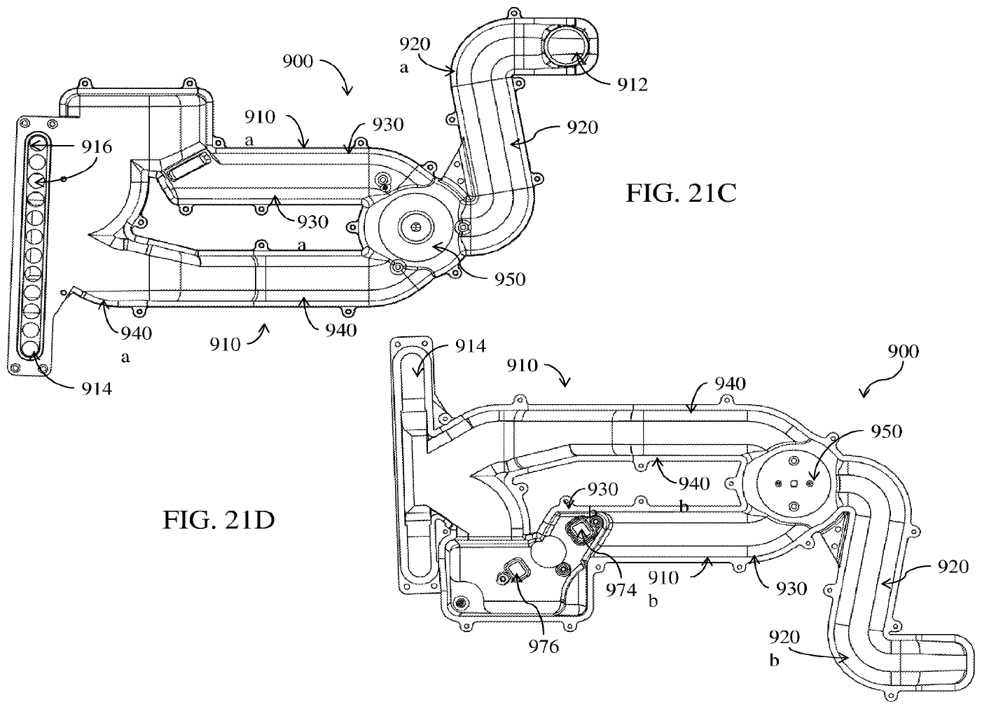

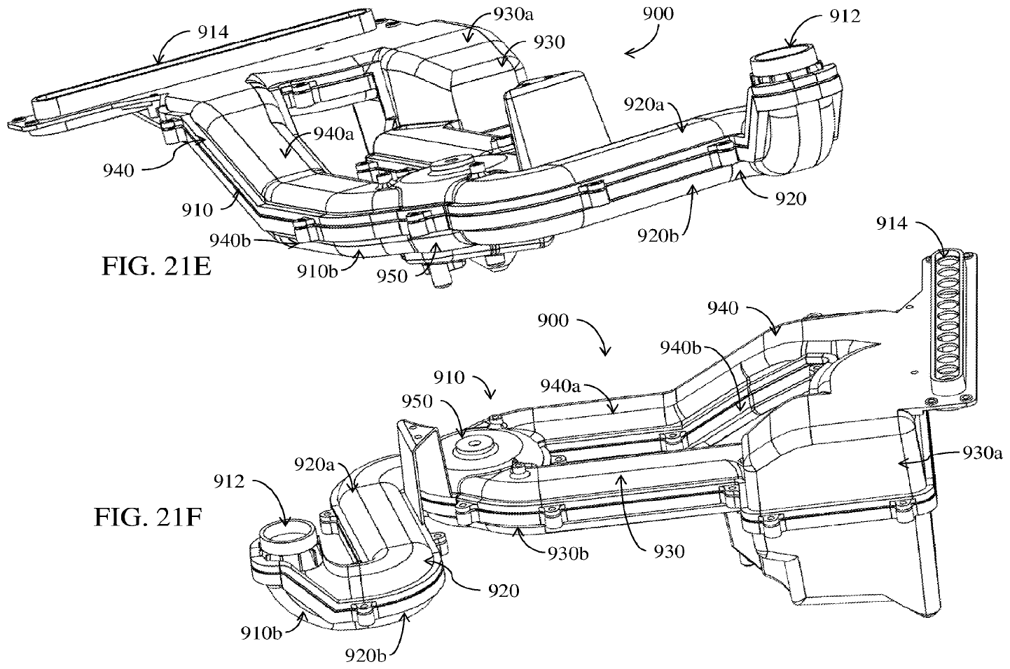

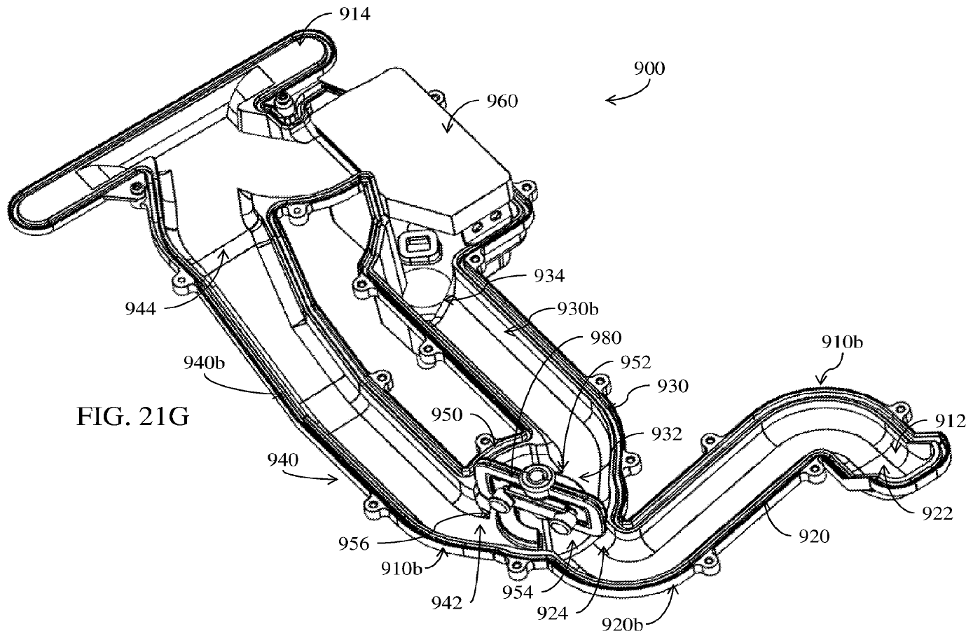

FIGS. 21A-21F illustrate a plurality of view of the evaporator 900. FIGS. 21A-21B illustrate a front and rear view of the evaporator 900, FIGS. 21C-21D illustrate a top and rear view of the evaporator 900, and FIGS. 21E and 21F illustrate perspective views of the evaporator 900.

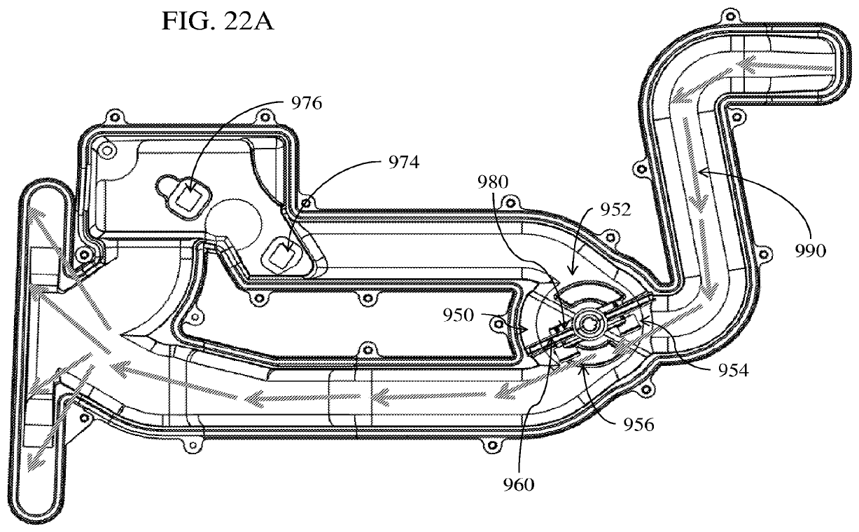

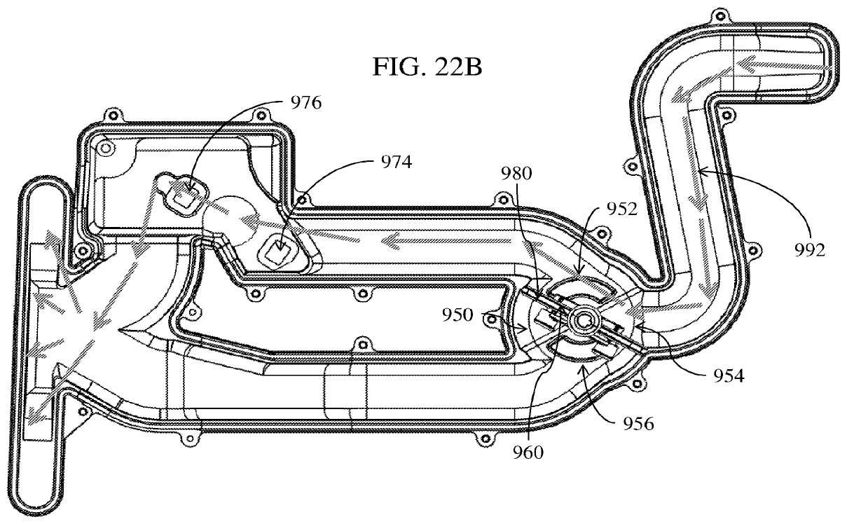

In some embodiments, the ducting 910 of the evaporator 900 includes a cap 910a and a base 910b. In some examples, the ducting 910 includes a first portion 920, a second portion 930, and a third portion 940. In some embodiments, the first portion 920, the second portion 930, and the third portion 940 are fluidly connected at a central hub 950. As shown in FIGS. 21G and 22A-22B, the central hub 950 can include an opening 952, an opening 954, and an opening 956. In some examples, the central hub 950 can secure a valve 980 such that the valve 980 can rotate between a first position and a second position.

In some examples, the first portion 920 can include a top portion 920a and a bottom portion 920b. The first portion 920 can extend between an inlet 912 and an outlet 924. In some embodiments, the outlet 924 is positioned in the top portion 920a and is configured to direct airflow from the outlet 714 of the ozone generation system 700 and into the opening 954 of the central hub 950.

In some embodiments, the second portion 930 can include a top portion 930a and a bottom portion 930b. The second portion 930 can extend between an inlet 932 and an outlet 934. In some embodiments, the second portion 930 can direct airflow out of the opening 952 of the central hub 950 and into the reservoir 970. As shown in FIG. 20B, a portion of the airflow is configured to move through the reservoir 970 along the fluid flow path 1032 and another portion of the airflow is configured to move through the nebulizer 1000. In some embodiments, the portion of airflow is configured to move out of the outlet 974 of the reservoir 970 and into an inlet 1010 of the nebulizer 1000. The airflow can move along the fluid flow path 1030 and out of the outlet 1020 of the nebulizer 1000 and back into the inlet 976 of the reservoir 970. The airflow along fluid flow path 1030 can mix with the airflow along fluid flow path 1032 to deliver disinfectant/sterilant out of the outlet 914 and into the inlet openings 160 of the chamber 100. In some embodiments, the outlet 914 has a width that corresponds with the plurality of inlet openings 160 to deliver airflow into the chamber 100.

In some embodiments, in the reservoir 970, the cross-section of the airflow can become larger. This can slow down the airflow and provide the droplets from the nebulizer 1000 additional time to evaporate. In some examples, the high surface area of mist droplets can ensure the machine runs at saturation levels of peroxide vapor through the whole machine.

In some examples, the third portion 940 can include a first portion 940a and a second portion 940b. The third portion 940 can extend between an inlet 942 and an outlet 944. In some embodiments, the third portion 940 can direct airflow out of the opening 956 of the central hub 950 and to the outlet 914. As mentioned above, in some embodiments, the outlet 914 is configured to deliver airflow into the inlet openings 160 of the chamber 100. In some examples, the outlet 914 has a width that corresponds with the plurality of the inlet openings 160 to deliver airflow into the chamber 100.

In some embodiments, the evaporator 900 includes a reservoir 970. The reservoir 970 can include an angled portion 978 that can serve as a collection point for excess disinfectant/sterilant. As will be discussed in more detail below, any collected disinfectant/sterilant can be pumped from the angled portion 978 of the reservoir 970 back into the nebulizer 1000 to allow the nebulizer 1000 to reuse the excess disinfectant/sterilant. In some embodiments, the reservoir 970 includes a body 972 with an outlet 974 and an inlet 976 in the base of the body 972. In some examples, the outlet 974 of the body 972 is configured to be fluidly connected to an inlet 1010 of the nebulizer 1000. In some embodiments, the inlet 976 of the body 972 is configured to be fluidly connected to the outlet 1020 of the nebulizer 1000.

FIG. 21G illustrates an embodiment of the evaporator 900 with the cap 910a of the ducting 910 removed to show the pad 960. In some examples, the pad 960 in the evaporator 900 can be positioned above the reservoir 970. In some embodiments, the pad 960 can stop most of the moisture (e.g., droplets) such that fluid flow moves from the reservoir 970 and out of the outlet 974 and into the chamber 100. In some examples, the evaporator pad 960 can capture droplets from the nebulizer 1000, whereas gas-phase disinfectant/sterilant (e.g., hydrogen peroxide) can pass right through the pad 960. In some embodiments, the pad 960 prevents excessive mist delivery of the hydrogen peroxide solution to the system. Excess mist circulating in the system, including in chamber 100, would lead to the formation of liquid pools in the system from excess condensate, which would be difficult to remove.

FIGS. 22A-22B illustrate a schematic of the fluid flow through the evaporator 900 with the cap 910a of the ducting 910 removed when the valve 980 is in two different positions. In some embodiments, the valve 980 is configured to rotate between approximately 60 degrees between the first position and the second position.

FIG. 22A illustrates the fluid flow 990 through the evaporator 900 when the valve 980 is in a first position. In some embodiments, a first end of the valve 980 is positioned adjacent to the inlet 932 of the second portion 930 and a second end of the valve 980 is positioned adjacent to a first side of the outlet 924 of the first portion 920. As shown the valve 980 allows the fluid flow 990 to move through the ducting 910 of the evaporator 900 to bypass the reservoir 970 and the nebulizer 1000. In some embodiments, the air flow is routed to bypass the reservoir 970 such that the air flows out of the outlet 914 and into the chamber 100 with no disinfectant/sterilant (e.g., hydrogen peroxide) added to it by the nebulizer 1000.

FIG. 22B illustrates the fluid flow 992 through the evaporator 900 when the valve 980 is in a second position. In some embodiments, a first end of the valve 980 is positioned adjacent to the inlet 942 of the third portion 940 and a second end of the valve 980 is positioned adjacent to a second side of the outlet 924 of the first portion 920. As shown, the valve 980 allows fluid flow 992 to move into the reservoir 970 and through the nebulizer 1000. In some embodiments, in the second position, the valve 980 routes the airflow into the reservoir 970 to deliver airflow out of the outlet 914 and into the chamber 100 that includes disinfectant/sterilant (e.g., hydrogen peroxide) in it.

### Nebulizer

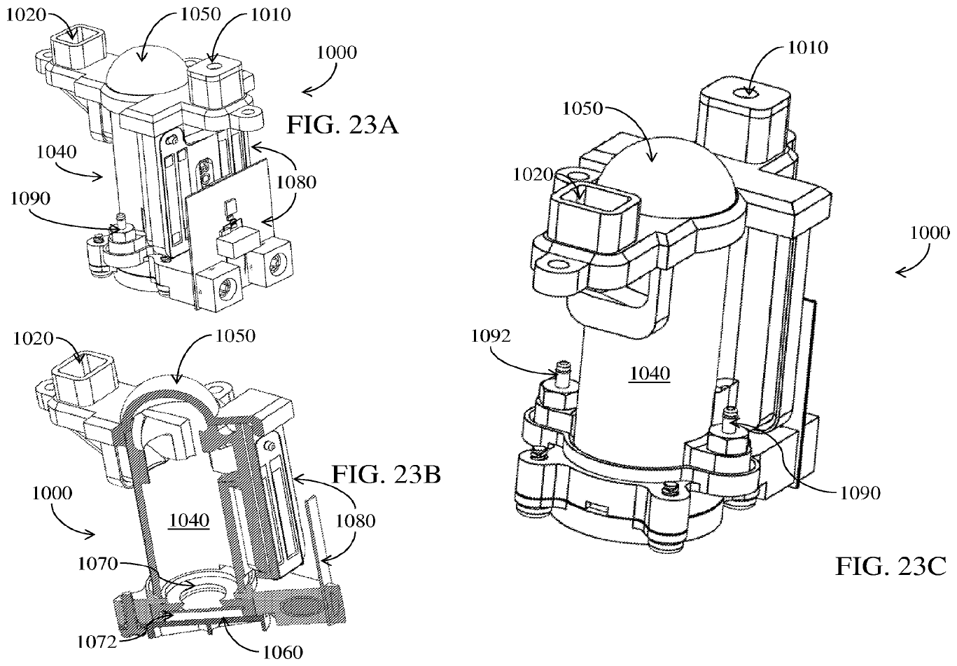

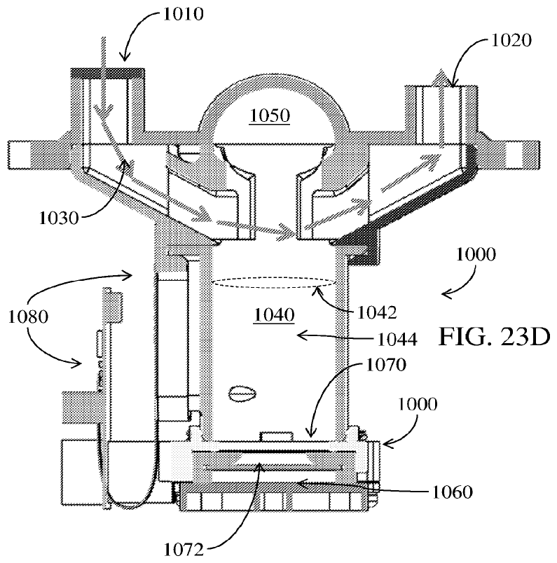

FIGS. 23A-23C illustrates an embodiment of the nebulizer 1000. In some embodiments, the nebulizer 1000 includes a chamber 1040 and a cap 1050 on the top of the chamber 1040. In some embodiments, the cap 1050 is dome shaped and has a curved inner surface. In some examples, the cap 1050 has a geometry (e.g., a dome) that recycles droplets of disinfectant/sterilant from the nebulization pool 1044 so that large droplets don't get stuck. In some embodiments, the domed cap 1050 catches large droplets and lets them run back down into the nebulization pool 1044. In some examples, the chamber 1040 includes a base surface 1070 comprising a piezocrystal 1060 and a seal 1072 positioned adjacent to the base surface 1070. The seal 1072 can be positioned in the base of the chamber 1040 to support the piezocrystal 1060 and prevent liquid from leaking out of the base surface 1070 of the nebulization pool 1044. In some embodiments, the nebulizer 1000 includes an inlet 1010 and an outlet 1020. As shown in FIGS. 20B and 23D, airflow is configured to flow into the inlet 1010, past a portion of the chamber 1040, and out of the outlet 1020. In some embodiments, the inlet 1010 is configured to receive airflow from the outlet 974 of the reservoir 970. In some examples, the outlet 1020 is configured to direct airflow out of the nebulizer 1000 and into the inlet 976 of the reservoir 970.

FIG. 23D illustrates a cross-sectional view of the nebulizer 1000 and the fluidly connected ozone generation system 700. In some embodiments, the chamber 1040 includes a nebulization pool 1044 that is maintained approximately at the liquid level 1042. In some embodiments, the nebulizer 1000 creates a mist that moves upwards and out into the reservoir 970 of the evaporator 900. In some embodiments, the piezocrystal 1060 vibrates in a megahertz range to generate a mist off the surface of the nebulization pool 1044 comprising peroxide. As shown in FIG. 23D, the fluid flow path 1030 flows across the chamber 1040 to catch the mist and flows into the reservoir 970 of the evaporator 900 where the fluid flow path 1030 mixes with the bulk airflow (e.g., fluid flow path 1032) in the evaporator 900. In some embodiments, the nebulization pool 1044 of the nebulizer 1000 is filled with a pool of peroxide up to a certain depth. As will be discussed in more detail below, the nebulizer 1000 can include a level sensor 1080 that can measure the liquid level 1042 of peroxide within the chamber 1040.

As mentioned above, the FIG. 20B illustrates an embodiment of the nebulizer 1000 fluidly attached to the evaporator 900. The fluid flow path 1032 indicates where most of the airflow entering the reservoir 970 of the evaporator 900 travels. The fluid flow path 1030 indicates where a portion of the airflow entering the reservoir 970 can travel through the nebulizer 1000. In some embodiments, a small percentage of air entering the reservoir 970 can travel along the fluid flow path 1030 through the nebulizer 1000. In some embodiments, the amount of airflow travelling along the fluid flow path 1030 can account for less than 0.5% of the airflow, less than 1.0% of the airflow, less than 1.5% of the airflow, less than 2.0% of the airflow, less than 2.5% of the airflow, less than 3.0% of the airflow, less than 3.5% of the airflow, less than 4.0% of the airflow, less than 4.5% of the airflow, less than 5.0% of the airflow, between 0.5% and 1.0% of the airflow, between 1.0% and 1.5% of the airflow, between 1.5% and 2.0% of the airflow, between 2.0% and 2.5% of the airflow, between 2.5% and 3.0% of the airflow, between 3.0% and 3.5% of the airflow, between 3.5% and 4.0% of the airflow, between 4.0% and 4.5% of the airflow, between 4.5% and 5.0% of the airflow, and any value in between those ranges listed, including endpoints. In some embodiments, the small amount of airflow travelling along the fluid flow path 1030 collects mist in the nebulizer 1000 and then mixes again with the main airflow along the fluid flow path 1032.

In some examples, the nebulizer 1000 generates mist using the piezocrystal 1060 positioned at the base surface 1070 of the chamber 1040 under the nebulization pool 1044 of disinfectant/sterilant. In some embodiments, the piezocrystal 1060 is configured to vibrate in the MHz range. In some examples, the piezocrystal 1060 is configured to vibrate at approximately 1.0 MHz, approximately 1.2 MHz, approximately 1.4 MHz, approximately 1.6 MHz, approximately 1.8 MHz, approximately 2.0 MHz, approximately 2.2 MHz, approximately 2.4 MHz, approximately 2.6 MHz, approximately 2.8 MHz, approximately 3.0 MHz, approximately 3.2 MHz, approximately 3.4 MHz, approximately 3.6 MHz, approximately 3.8 MHz, approximately 4.0 MHz, between 1.0-1.2 MHz, between 1.2-1.4 MHz, between 1.4-1.6 MHz, between 1.6-1.8 MHz, between 1.8-2.0 MHz, between 2.0-2.2 MHz, between 2.2-2.4 MHz, between 2.4-2.6 MHz, between 2.6-2.8 MHz, between 2.8-3.0 MHz, between 3.0-3.2 MHz, between 3.2-3.4 MHz, between 3.4-3.6 MHz, between 3.6-4.0 MHz, and any value in between those ranges listed, including endpoints.

In some embodiments, the vibration caused by the piezocrystal 1060 can create a mist from the liquid in the nebulization pool 1044. In some examples, mist and large droplets fly off the surface of the liquid in the nebulization pool 1044. In some embodiments, large droplets fly exclusively upward where they can hit the underside of the cap 1050. As discussed above, the curved surface of the cap 1050 allow the droplets to fall back down into the nebulization pool 1044. In some embodiments, the mist generated by the piezocrystal 1060 can be carried by airflow through the nebulizer 1000 along the fluid flow path 1030. In some embodiments, the mist particles have a size of approximately 0.1 μm, approximately 0.2 μm, approximately 0.3 μm, approximately 0.4 μm, approximately 0.5 μm, approximately 0.6 μm, approximately 0.7 μm, approximately 0.8 μm, approximately 0.9 μm, approximately 1.0 μm, approximately 1.1 μm, approximately 1.2 μm, approximately 1.3 μm, approximately 1.4 μm, approximately 1.5 μm, approximately 1.6 μm, approximately 1.7 μm, approximately 1.8 μm, approximately 1.9 μm, approximately 2.0 μm, approximately 2.1 μm, approximately 2.2 μm, approximately 2.3 μm, approximately 2.4 μm, approximately 2.5 μm, approximately 2.6 μm, approximately 2.7 μm, approximately 2.8 μm, approximately 2.9 μm, approximately 3.0 μm, approximately 3.1 μm, approximately 3.2 μm, approximately 3.3 μm, approximately 3.4 μm, approximately 3.5 μm, approximately 3.6 μm, approximately 3.7 μm, approximately 3.8 μm, approximately 3.9 μm, approximately 4.0 μm, approximately 4.1 μm, approximately 4.2 μm, approximately 4.3 μm, approximately 4.4 μm, approximately 4.5 μm, approximately 4.6 μm, approximately 4.7 μm, approximately 4.8 μm, approximately 4.9 μm, approximately 5.0 μm, approximately 5.1 μm, approximately 5.1 μm, approximately 5.2 μm, approximately 5.3 μm, approximately 5.4 μm, approximately 5.5 μm, approximately 5.6 μm, approximately 5.7 μm, approximately 5.8 μm, approximately 5.9 μm, approximately 6.0 μm, approximately 6.5 μm, approximately 7.0 μm, approximately 7.5 μm, approximately 8.0 μm, approximately 8.5 μm, approximately 9.0 μm, approximately 9.5 μm, approximately 10.0 μm, approximately 10.5 μm, approximately 11.0 μm, approximately 11.5 μm, approximately 12.0 μm, approximately 12.5 μm, approximately 13.0 μm, approximately 13.5 μm, approximately 14.0 μm, approximately 14.5 μm, approximately 15.0 μm, approximately 15.5 μm, approximately 16.0 μm, approximately 16.5 μm, approximately 17.0 μm, approximately 17.5 μm, approximately 18.0 μm, approximately 18.5 μm, approximately 19.0 μm, approximately 19.5 μm, between 0.0-0.5 μm, between 0.5-1.0 μm, between 1.0-1.5 μm, between 1.5-2.0 μm, between 2.0-2.5 μm, between 2.5-3.0 μm, between 3.0-3.5 μm, between 3.5-4.0 μm, between 4.0-4.5 μm, between 4.5-5.0 μm, between 5.0-5.5 μm, between 5.5-6.0 μm, between 6.0-7.0 μm, between 7.0-8.0 μm, between 8.0-9.0 μm, between 9.0-10.0 μm, between 10.0-11.0 μm, between 11.0-12.0 μm, between 12.0-13.0 μm, between 13.0-14.0 μm, between 14.0-15.0 μm, between 15.0-16.0 μm, between 16.0-17.0 μm, between 17.0-18.0 μm, between 18.0-19.0 μm, between 19.0-20.0 μm, between 0.0-2.5 μm, between 2.5-5.0 μm, between 0.5-5.0 μm, between 5.0-10.0 μm, between 10.0-15.0 μm, between 15.0-20.0 μm, and any value in between those ranges listed, including endpoints.

In some embodiments, the nebulizer 1000 includes a level sensor 1080 to detect the liquid level 1042 in the nebulization pool 1044 and validate that we have nebulized the proper amount. In some examples, the level sensor 1080 is a capacitive sensor. In some embodiments, the system 15 is configured to stop nebulizing to check the liquid level 1042 in the nebulization pool 1044, before beginning nebulizing again to ensure the proper amount of liquid is nebulized by volume.

In some examples, the nebulization pool 1044 is filled by first drawing out any remaining liquid through the collection point pump fitting 1092 from the angled collection point portion 978 before the nebulization pool 1044 is topped off to the proper liquid level 1042 from the cartridge 400 through the cartridge pump fitting 1090. In some embodiments, the piezocrystal 1060 makes mist until the liquid level 1042 has dropped using a preset scheme.

In some embodiments, a “nebulization cone” forms on the surface of the liquid of the nebulization pool 1044. The liquid surface can form a shape that is wide at the bottom and narrow at the top (e.g., a horn, a trumpet, a bell shape). In some embodiments, both large droplets and mist can come off this surface of the nebulization pool 1044. The control of the nebulization cone can be important for good performance. If the cone collapses, mist generation can slow dramatically and/or stop. In some embodiments, cone collapses can be caused when there is too much airflow across it, blowing it over, or having too many droplets falling back down and splashing the surface of the cone collapse. In some embodiments, the cap 1050 is configured to direct large droplets back down along the surfaces of the chamber 1040 instead of falling down onto the surface of the nebulization pool 1044. In some examples, this can prevent the cone from being disturbed.

The nebulization cone can be important for nebulizer control. In some embodiments, the nebulization cone takes a large amount of liquid to form (e.g., 1 mL) which can cause the liquid level 1042 to drop. As the liquid level 1042 can continue to drop as mist is carried away, the piezocrystal 1060 can preemptively stop vibrating when the liquid level 1042 dips below a preset threshold level. However, this can cause the cone to collapse and cause the liquid level 1042 to rise. In some embodiments, the liquid level 1042 can rise at the end of nebulization more than it fell at the start of nebulization. This can be caused because a larger cone can form between a lower liquid level 1042 and the cap 1050. The previously described challenges make controlling the amount of mist generated accurately difficult. In some embodiments, the system 15 includes two algorithms to control the mist generation amounts. In some embodiments, an algorithm is provided to predict the difference between level drop due to cone formation and level rise due to cone collapse. The system 15 can start nebulizing, measure the starting liquid level 1042 immediately after the nebulization cone forms, and stop nebulizing when the liquid level 1042 drops to a targeted amount plus the predicted amount. In some embodiments, an algorithm is provided to measure the liquid level 1042 prior to nebulizing, nebulize for an amount of time less than the required time to nebulize the target amount, stop nebulizing, and checking the liquid level 1042 within the chamber 1040.

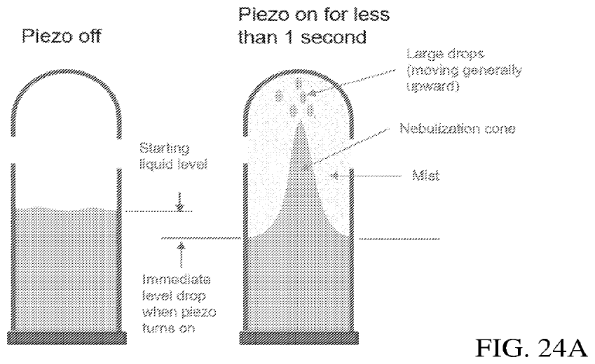

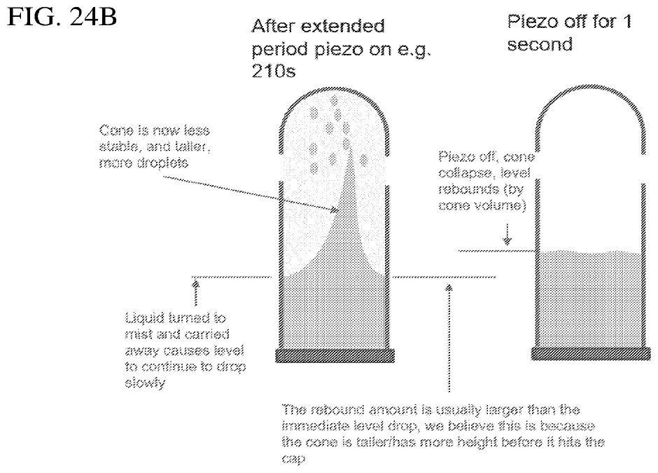

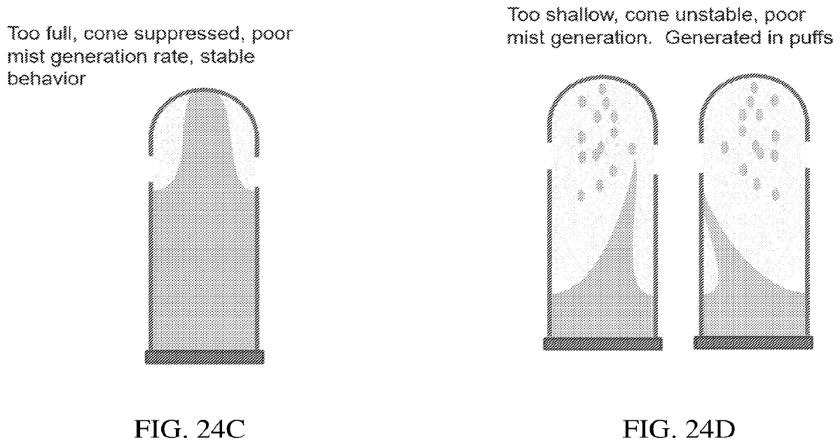

FIGS. 24A-24B illustrates cavitation behavior within the nebulizer 1000. FIG. 24A illustrates a schematic of the liquid level of the nebulization pool within the chamber and the cap of the nebulizer 1000 when the piezocrystal 1060 is turned off and when the piezocrystal 1060 is turned on for less than a second. As shown, when the piezocrystal 1060 is turned on briefly for less than a second, large droplets move upwards towards the dome and a nebulization cone forms along with mist. With the upward movement of the liquid to form the nebulization cone, the overall liquid level of the nebulization pool drops. FIG. 24B illustrates a schematic of the liquid level of the nebulization pool within the chamber and the cap of the nebulizer 1000 when the piezocrystal 1060 has been turned on for an extended period of time (e.g., 210 seconds) and after the piezocrystal 1060 has been turned off for 1 second. As shown, when the piezocrystal 1060 is turned on for an extended period time, the nebulization cone formed is less stable than the nebulization cone shown in FIG. 24A when the piezocrystal 1060 was initially turned on. After an extended period of time, the nebulization cone can be taller and form more droplets. As shown, as the liquid is being turned into mist and carried out of the nebulizer 1000, the liquid level within the chamber continues to drop as the piezocrystal 1060 is operated. Once the piezocrystal 1060 is turned off, with the collapse of the nebulization cone, the liquid level rebounds (e.g., by the cone volume) within the chamber. In some embodiments, the rebound amount is larger than the immediate level drop because the nebulization cone is taller/has more height before it hits the cap.

FIGS. 24C-24D illustrates the effect that the distance between the cap and the liquid level has on the nebulization cone. In some examples, if the cap 1050 is positioned too high above the liquid level, droplets falling back down onto the nebulization pool 1044 can generate excessive energy that disturbs the nebulization cone. This can be seen in the schematic of FIG. 24D. As shown, the liquid level is too shallow, the cone becomes unstable and there is poor mist generation. In some embodiments, this results in puffs being generated. In some embodiments, if the cap 1050 is too close to the liquid level, it can suppress the rate of mist generation. In some examples, if the liquid level 1042 above the piezocrystal 1060 is too low, mist production can also be slowed. This can be seen in the schematic of FIG. 24C. As shown, the chamber of the nebulizer 1000 is too full which can result in a suppressed nebulization, and stable behavior that results in a poor mist generation rate.

In some embodiments, the nebulizer 1000 includes a cartridge pump fitting 1090 and a collection point pump fitting 1092. In some embodiments, the cartridge pump fitting 1090 is positioned on a first side of the nebulizer 1000 and the collection point pump fitting 1092 is positioned on a second side of the nebulizer 1000. In some examples, the cartridge pump fitting 1090 is fluidly connected to the cartridge 400. In some embodiments, the collection point pump fitting 1092 is fluidly connected to the angled collection point portion 978 of the reservoir 970. In some embodiments, the pool of sterilant/disinfectant within the chamber 1040 of the nebulizer 1000 (i.e., nebulization pool 1044) can refill the nebulization pool 1044 by first drawing from the angled collection point portion 978 of the reservoir 970. In some examples, the chamber 1040 can refill the nebulization pool 1044 with drawing additional disinfectant/sterilant from the cartridge 400 through the cartridge pump fitting 1090. This can increase the efficiency of the evaporator 900 and provide for reuse of the disinfectant/sterilant.

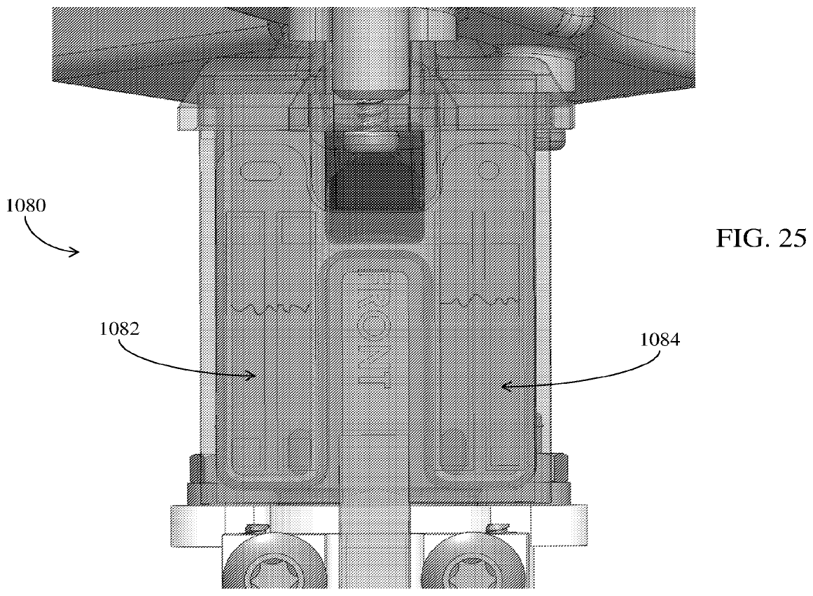

FIG. 25 illustrates an embodiment of the level sensor 1080. As mentioned above, the level sensor 1080 can be a capacitive type. In some embodiments, the level sensor 1080 can include three (3) capacitors. In some examples, one of the capacitors always sees liquid, one of the capacitors always sees air, and one of the capacitors is partially covered. In some embodiments, the level sensor 1080 includes two reference capacitors 1082 and a sense capacitor 1084. In some examples, the two reference capacitors 1082 and the sense capacitor 1084 are connected by ports to the bottom of the nebulization pool 1044. In some examples, the air above the liquid level 1042 and the air above the sense capacitor are connected to balance pressures.

In some embodiments, the nebulizer fluidly connected with the evaporator 900 can have any structure or be of any type that provides mist generation/nebulization. In some examples, the nebulizer can be a mesh nebulizer. The mesh nebulizer can include a metal plate with openings of 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, between 2-5 μm, between 5-10 μm, between 10-15 μm, between 15-20 μm, between 2-10 μm, between 10-20 μm, between 2-20 μm, and any value in between those ranges listed, including endpoints. The mesh nebulizer can also include a plate that is vibrated by a piezo crystal at a frequency of 50 MHz, 55 MHz, 60 MHz, 65 MHz, 70 MHz, 75 MHz, 80 MHz, 85 MHz, 90 MHz, 95 MHz, 100 MHz, 105 MHz, 110 MHz, 115 MHz, 120 MHz, 125 MHz, 130 MHz, 135 MHz, 140 MHz, 145 MHz, 150 MHz, 155 MHz, 160 MHz, 165 MHz, 170 MHz, 175 MHz, 180 MHz, 185 MHz, 190 MHz, 200 MHz, between 50-75 MHz, between 75-100 MHz, between 100-125 MHz, between 125-150 MHz, between 150-175 MHz, between 175-200 MHz, and any value in between those ranges listed, including endpoints. The mesh nebulizer can include a liquid disinfectant/sterilant on one side of the nebulizer and generate mist on the other.

In some embodiments, the nebulizer fluidly connected with the 900 can be a jet nebulizer. The jet nebulizer can use a high-speed jet of air to create a mist out of a liquid disinfectant/sterilant.

System for Disinfection, Sterilization, and/or Sanitization

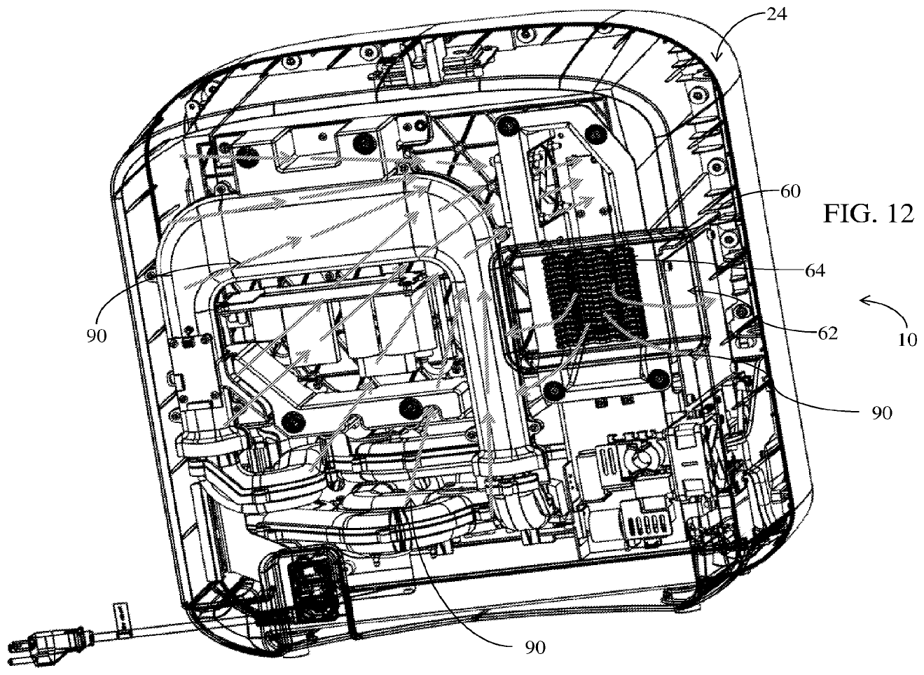

As an overview, in some embodiments, air is configured to flow through the inlet 232 of the filter 230 and into the inlet 832 of the ducting 800. In some examples, airflow can then move through the outlet 844 of the ducting 800 and into the inlet 712 of the ozone generation system 700. In some embodiments, air flows past the ozone generator 740 and out of the outlet 714 of the ozone generation system 700. In some examples, the airflow can travel into the inlet 912 and enter the evaporator 900. In some embodiments, depending on the position of the valve 980, airflow can travel either through the nebulizer 1000 or bypass the nebulizer 1000 entirely. In some examples, airflow can enter the outlet 914 of the evaporator 900 and travel through the inlet openings 160 of the chamber 100. In some embodiments, once airflow has entered the chamber 100, it can flow through the openings 124 of the inlet plate 120, across the chamber to disinfect and/or sterilize an item placed in the chamber 100, and back out of the openings 134 of the outlet plate 130. In some examples, airflow can exit the chamber 100 through the outlet openings 170 and into the inlet 872 of the adaptor 870. In some embodiments, the airflow can move from the inlet 872 of the adaptor 870 and out of the outlet 874 of the adaptor 870 and into the blower 860. In some examples, airflow can move into the inlet 882 of the ducting 800 through the blower 860 and travel through the muffler 880 of the ducting 800. In some embodiments, depending on the position of the valve 805, airflow can bypass the outlet 824 of the ducting 800 and continue to the ozone generation system 700. Alternatively, in some examples, depending on the position of the valve 805, airflow can exit out of the outlet 824 and through the inlet 242 of the outlet filter 240 and out of the outlet 244 filter 240 and into the housing 20 of the device 10. As illustrated in FIG. 12, airflow is then blown across the ambient ozone sensor 520 before it exits through the vent 64 of the exhaust 60. As previously discussed, if the amount of ozone detected at the ambient ozone sensor 520 is above a threshold, the system 15 of the device 10 will shut down to prevent unsafe levels of ozone from exiting the device 10 and into the surrounding environment. In some embodiments, airflow is configured to flow through the inlet of the nebulizer 1000, past portion of the nebulizer chamber and through the evaporator pad 960 and out of the evaporator 900.

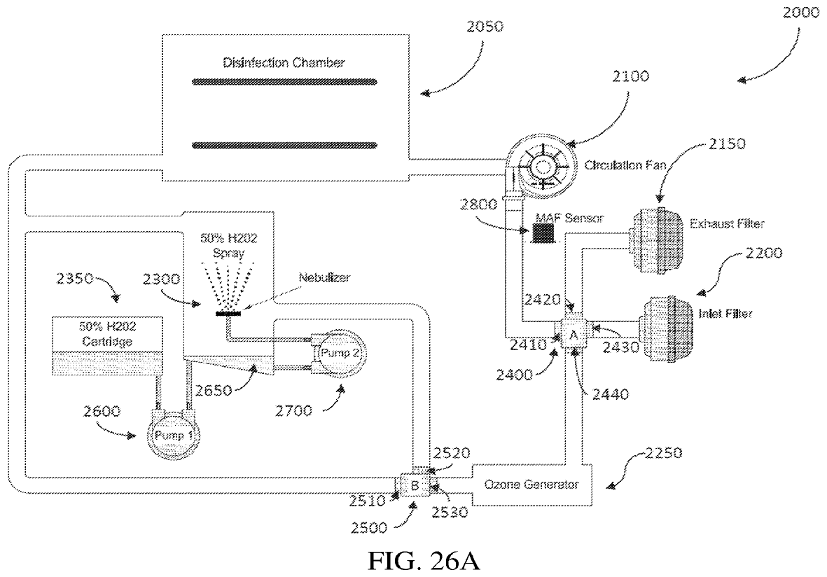

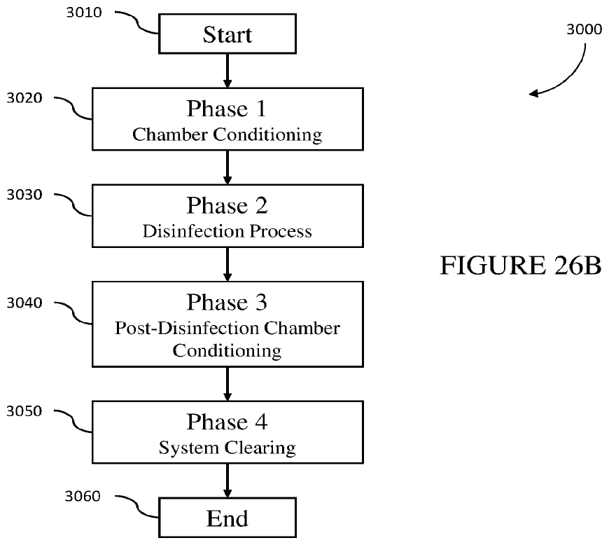

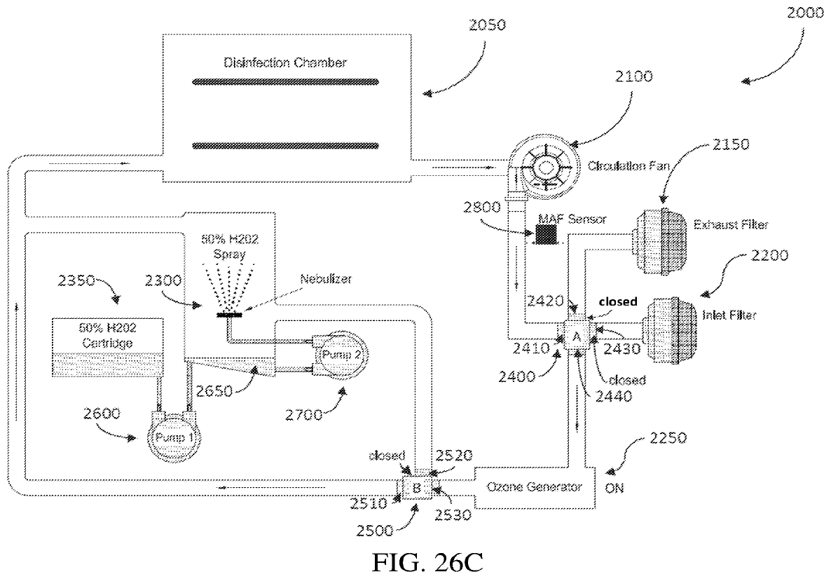

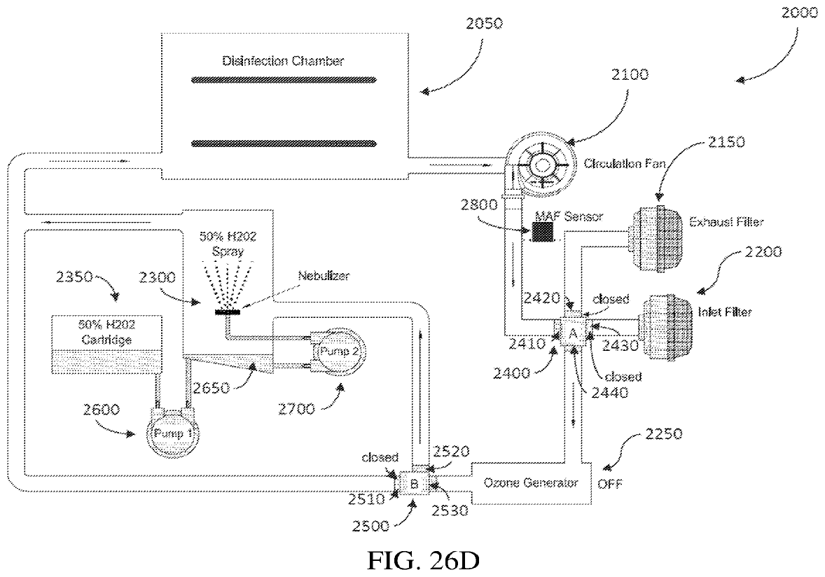

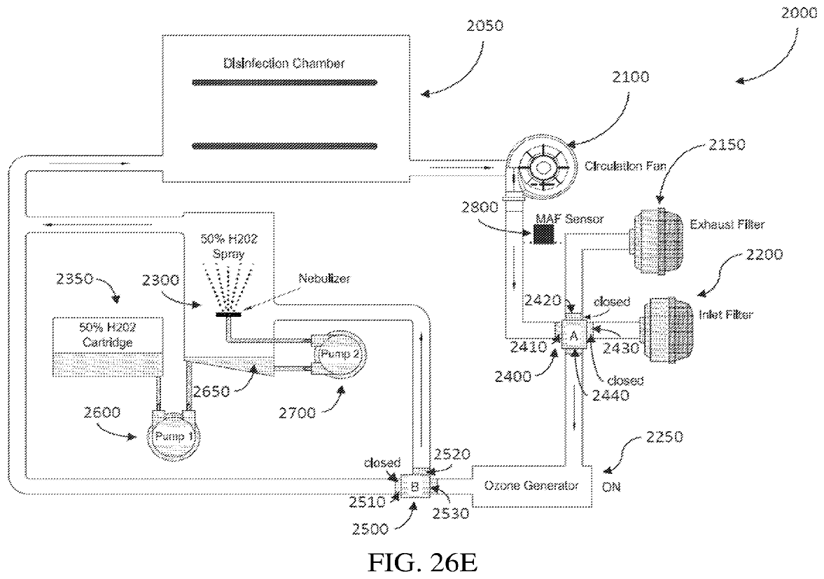

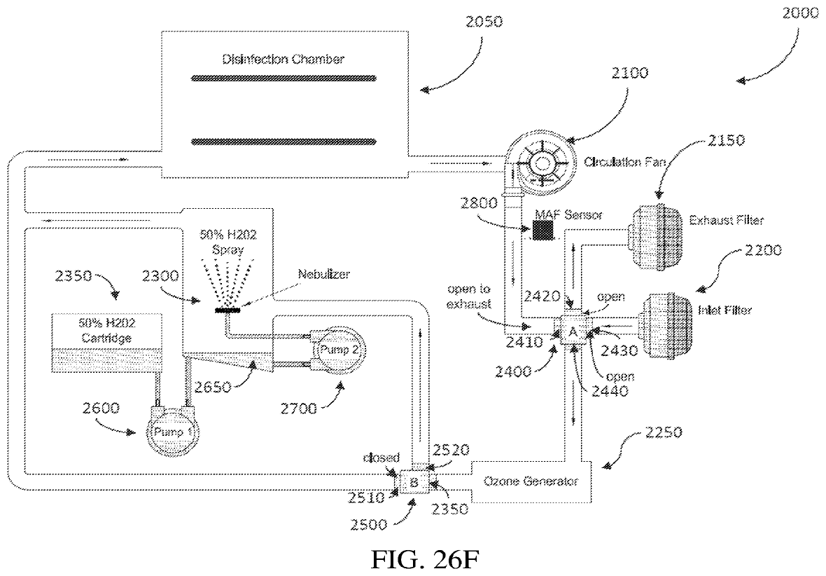

FIG. 26 illustrates a schematic diagram of an embodiment of a system 2000 for reducing microorganisms on a surface. In some examples, the system 2000 of FIG. 26 correlates with the system 15 of the device 10. In some embodiments, the system 2000 can be a fully automated and integrated system which can include a chamber 2050 (e.g., the chamber 100 of the device 10), an ozone generator 2250 (e.g., the ozone generation system 700 of the device 10), a nebulizer 2300 (e.g., the nebulizer 1000 of the device 10), at least one pump (i.e., pump 2600 or pump 2700), a circulating fan 2100 (e.g., the blower 860 of the device 10), a plurality of valves (i.e., valve 2400 and valve 2500), an inlet and inlet filter 2200 (e.g., the inlet filter 230), and an exhaust with an exhaust filter 2150 (e.g., the outlet filter 240). In some embodiments, the system 2000 can include a sensor 2800. In some embodiments, the sensor 2800 is a flow sensor that is configured to measure leaks in the system 15.

In some embodiments, the at least one pump 2600, 2700 can be a peristaltic pump or other precision pump. In some examples, the sensor 2800 can be a mass airflow sensor. The sensor 2800 can measure the flow rate through the system 2000 to ensure consistent flow rate through the system 2000 regardless of the path of fluid flow. In some embodiments, the sensor 2800 can detect leak rate. For examples, the fluid flow through the nebulizer 2300, the pump 2600, and the pump 2700 can experience a different level of resistance than when the fluid flow bypasses the nebulizer 2300 and the two pump 2600, 2700.

As will be discussed in more detail below, the system 2000 can be designed to draw a precise volume between about 1.5 mL and 2.5 mL (e.g., 1.9 mL, 2.0 mL, 2.1 mL, 2.2 mL, 2.3 mL, 2.4 mL, or 2.5 mL) of liquid disinfectant (i.e., 50% H₂O₂) from the cartridge by the at least one pump 2600, 2700, nebulize the liquid into a spray (i.e. mist) using the nebulizer 1000, and transport the spray via forced air into a continuous closed loop flow through the chamber 100. In some embodiments, the system 2000 can be designed to draw a volume of 1.6 mL, 1.7 mL, 1.8 mL, 1.9 mL, 2.0 mL, 2.1 mL, 2.2 mL, 2.3 mL, 2.4 mL, 2.5 mL, between about 1.6 mL-1.7 mL, between about 1.7 mL-1.8 mL, between about 1.8 mL-1.9 mL, between about 1.9 mL-2.0 mL, between about 2.0 mL-2.1 mL, between about 2.1 mL-2.2 mL, between about 2.2 mL-2.3 mL, between about 2.3 mL-2.4 mL, between about 2.4 mL-2.5 mL and any value in between those ranges listed, including endpoints. The disinfectant spray can be contact the surfaces of the items placed in the chamber 100 to inactivate the pathogens during the disinfectant cycle. In some embodiments, the system 2000 can be designed to draw a precise volume between about 1.0 mL and 2.5 mL (e.g., 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, or 2.5 mL) of liquid disinfectant (i.e., 50% H₂O₂) from the cartridge by the at least one pump 2600, 2700, nebulize the liquid into a spray using the nebulizer 1000, and transport the spray via forced air into a continuous closed loop flow through the chamber 100. In some embodiments, the system 2000 can be designed to draw a volume of 1.0 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, between about 0.9 mL-1.1 mL, between about 1.0 mL-1.2 mL, between about 1.8 mL-1.9 mL, between about 1.9 mL-2.0 mL, between about 2.0 mL-2.1 mL, between about 2.1 mL-2.2 mL, between about 2.2 mL-2.3 mL, between about 2.3 mL-2.4 mL, between about 2.4 mL-2.5 mL and any value in between those ranges listed, including endpoints. In some embodiments, the disinfectant spray (i.e. mist) produced at nebulizer 1000 reaches the evaporator pad 960, wherein the spray is converted to vapor. The disinfectant spray and the vapor that is produced in the evaporator pad 960 can be in contact with the surfaces of the items placed in the chamber 100 to inactivate the pathogens during the disinfectant cycle. In some embodiments, the evaporator pad 960 prevents excessive mist delivery of the hydrogen peroxide solution to the system. In some embodiments, the system 2000 can be designed to draw a precise volume such that when nebulized, renders the internal humidity within the chamber to about 100%. In some embodiments, the system 2000 can be designed to draw a precise volume such that when nebulized, renders the internal humidity within the chamber to about above 80%, above 90%, above 95%, or any value in between the aforementioned. In some embodiments, the system 2000 can be designed to draw a precise volume such that when nebulized, renders the internal humidity within the chamber to between about 20%, 25%, 30%, 35%, 40%, 45%, 50%, 55%, 60%, 65%, 70%, 75%, 80%, 85%, 90%, 95%, 100%, or any value in between.

The system 2000 can include an ozone generator 2250. In some embodiments, the ozone generator 2250 can produce ozone. The ozone produced can be used in two ways in the system 2000. In some embodiments, the ozone produced by the ozone generator 2250 can precondition the chamber 100. In some examples, the ozone produced by the ozone generator 2250 can neutralize the residual H₂O₂ after the item placed in the chamber 100 has been disinfected.

In some embodiments, the system 2000 includes a reservoir 2650 (e.g., the reservoir 970) between the pump 2600 and the pump 2700. The pump 2600 can be preprogrammed to pump out a predetermined amount of disinfectant/sterilant until the cartridge 2350 (e.g., the cartridge 400) is empty. Because excess disinfectant/sterilant (e.g., H₂O₂) is removed after each disinfection/sterilization cycle, the pump 2700 can be responsible for delivering the proper amount of disinfectant/sterilant (e.g., H₂O₂) to the nebulizer 2300. Any excess amount of disinfectant/sterilant (e.g., H₂O₂) can be stored in the reservoir 2650. In some embodiments, the reservoir 2650 has a predetermined amount of disinfectant/sterilant (e.g., H₂O₂) that is stored; if the amount of disinfectant/sterilant (e.g., H₂O₂) falls below the predetermined amount, pump 2600 will pump out disinfectant/sterilant to fill the reservoir 2650 to the proper amount. In some examples, the volume stored in the reservoir 2650 is the amount of disinfectant/sterilant that is delivered to the nebulizer 2300. The pump 2600 can be programmed to pump whatever is in the reservoir 2650. However, if there is any disinfectant/sterilant unused during disinfection/sterilization, the disinfectant/sterilant will be stored in the reservoir 2650. When the amount of disinfectant/sterilant of the reservoir 2650 falls below the predetermined volume, the pump 2600 will pump disinfectant/sterilant out of the cartridge 2350 to fill the reservoir 2650 to the predetermined volume.

FIG. 26A illustrates a schematic diagram of an embodiment of a system 2000 for reducing microorganisms on a surface. As illustrated, the system 2000 can be a fully automated and integrated system which can include a chamber 2050, an ozone generator 2250, a nebulizer 2300, at least one pump (i.e., pump 2600 or pump 2700), a circulating fan 2100, a plurality of valves (i.e., valve 2400, and valve 2500), an inlet and inlet filter 2200, and an exhaust filter 2150. In some embodiments, the system 2000 can include a sensor 2800.

In some embodiments, the at least one pump 2600, 2700 can be a peristaltic pump or other precision pump.

The sensor 2800 can be a mass airflow sensor. The sensor 2800 can measure the flow rate through the system 2000 to ensure consistent flow rate through the system 2000 regardless of the path of fluid flow. For example, fluid flow through the nebulizer 2300, the pump 2600, and the pump 2700 can experience a different level of resistance than when the fluid flow bypasses the nebulizer 2300 and the two pumps 2600, 2700. The sensor 2800 can measure the flow rate and adjust the power delivered to the circulating fan 2100 accordingly.

As will be discussed in more detail below, the system 2000 can be designed to draw a precise volume between about 1.5 mL and 2.5 mL (e.g., 1.9 mL, 2.0 mL, 2.1 mL, 2.2 mL, 2.3 mL, 2.4 mL, or 2.5 mL) of liquid disinfectant (i.e., 50% H₂O₂) from the cartridge by the at least one pump 2600, 2700, nebulize the liquid into a spray using the nebulizer 2300, and transport the spray via forced air into a continuous closed loop flow through the disinfection chamber 2050. In some embodiments, the system 600 can be designed to draw a volume of 1.6 mL, 1.7 mL, 1.8 mL, 1.9 mL, 2.0 mL, 2.1 mL, 2.2 mL, 2.3 mL, 2.4 mL, 2.5 mL, between about 1.6 mL-1.7 mL, between about 1.7 mL-1.8 mL, between about 1.8 mL-1.9 mL, between about 1.9 mL-2.0 mL, between about 2.0 mL-2.1 mL, between about 2.1 mL-2.2 mL, between about 2.2 mL-2.3 mL, between about 2.3 mL-2.4 mL, between about 2.4 mL-2.5 mL and any value in between those ranges listed, including endpoints. The disinfectant spray can be contact the surfaces of the items placed in the disinfection chamber 2050 to inactivate the pathogens during the disinfectant cycle.

The system 2000 can include an ozone generator 2250. In some embodiments, the ozone generator 2250 can produce ozone. The ozone produced can be used in to ways in the system 2000. In some embodiments, the ozone produced by the ozone generator 2250 can precondition the disinfection chamber 2050. In some examples, the ozone produced by the ozone generator 2250 can neutralize the residual H₂O₂ after the item placed in the disinfection chamber 2050 has been disinfected.

As shown in FIGS. 26C-26F, the system 2000 includes a reservoir 2650 between the pump 2600 and the pump 2700. The pump 2600 can be preprogrammed to pump out a predetermined amount of disinfectant/sterilant until the cartridge 2350 is empty. Because excess disinfectant/sterilant (e.g., H₂O₂) is removed after each disinfection/sterilization cycle, the pump 2700 can be responsible for delivering the proper amount of disinfectant/sterilant (e.g., H₂O₂) to the nebulizer 2300. Any excess amount of disinfectant/sterilant (e.g., H₂O₂) can be stored in the reservoir 2650. In some embodiments, the reservoir 2650 has a predetermined amount of disinfectant/sterilant (e.g., H₂O₂) that is stored; if the amount of disinfectant/sterilant (e.g., H₂O₂) falls below the predetermined amount, pump 2600 will pump out disinfectant/sterilant to fill the reservoir 2650 to the proper amount. In some examples, the volume stored in the reservoir 2650 is the amount of disinfectant/sterilant that is delivered to the nebulizer 2300. The pump 2700 can be programmed to pump whatever is in the reservoir 2650. However, if there is any disinfectant/sterilant unused during disinfection/sterilization, the disinfectant/sterilant will be stored in the reservoir 2650. When the amount of disinfectant/sterilant of the reservoir 2650 falls below the predetermined volume, the pump 660 will pump disinfectant/sterilant out of the cartridge 2350 to fill the reservoir 2650 to the predetermined volume.

As provided in the table below, the system 2000 can be configured to disinfect an item. In some embodiments, the system 2000 operates at ambient temperature and ambient pressure conditions in a continuous closed loop flow through the cycle. In some embodiments, the system 2000 can operate at a temperature of 20° C., 21° C., 22° C., 23° C., 24° C., 25° C. In some embodiments, the system 2000 can operate at a temperature between about 20° C. and 21° C., between about 21° C. and 22° C., between about 22° C. and 23° C., between about 23° C. and 24° C., and between about 24° C. and 25° C. As discussed above, the disclosed system 2000 can operate to disinfect without the use of a heater.

In some examples, the system 2000 can operate with a relative humidity of 20%, 25%, 30%, 35%, 40%, 45%, 50%, 55%, and 60%. In some embodiments, the relative humidity is an ambient relative humidity. In some embodiments, the system 2000 can operate with a relative humidity of between about 20% and 25%, between about 25% and 30%, between about 30% and 35%, between about 35% and 40%, between about 40% and 45%, between about 45% and 50%, between about 50% and 55%, between about 55% and 60%. As discussed above, embodiments of the system 2000 do not include a dehumidifier remove moisture from the system 2000. The below table provides a summary of an embodiment of the range of external operating conditions of the system 2000:

| Operating Conditions | Minimum | Maximum |
|---|---|---|
| Temperature | 20° C. | 25° C. |
| Relative Humidity | 20% | 60% |

In some embodiments, the system 2000 is isolated environmentally, including temperature and relative humidity using insulation and physical barriers. In some embodiments, the system 2000 is isolated environmentally, such that the internal operating conditions are at equilibrated at a preset range of temperature and humidity values. In some embodiments, the internal temperature of the walls of chamber 100 are kept at 45+/−5 centigrade. In some embodiments, the internal temperature of the walls of chamber 100 are kept at, for example, 30, 35, 40, 45, 50, 55, or 60 degrees centigrade, or any value in between. In some embodiments, the system 2000 is isolated so that ambient operating conditions are lower than from 20° C. In some embodiments, the system 2000 is isolated so that ambient operating conditions are higher than from 25° C. In some embodiments, the system 2000 is isolated so that ambient operating conditions are lower than from 20% relative ambient humidity. In some embodiments, the system 2000 is isolated so that ambient operating conditions are higher than from 60% relative ambient humidity.

FIG. 26B illustrates a flowchart of a non-limiting method for disinfection 3000. As illustrated in the flowchart, the method 3000 can start at step 3010, proceed to Phase 1 at step 3020, proceed to Phase 2 at step 3030, proceed to Phase 3 at step 3040, proceed to Phase 4 at step 3050, and end at step 3060. Each of these phases will be discussed in more detail below. In some embodiments, the process time for the method 3000 is 10 minutes and includes four distinct phases. In some examples, the contact time with the disinfectant (i.e., H₂O₂) is approximately 4.5 minutes of the 10-minute process. A summary of the time for each phase of an embodiment of method 700 is provided below:

| | | Phase | Elapsed |
|---|---|---|---|
| | | Duration | Time |
| Phase | Description | (min) | (min) |
| Phase 1 | Chamber Conditioning | 2.5 | 0-2.5 |
| Phase 2 | Disinfection Process (contact time) | 4.5 | 2.5-7.0 |
| Phase 3 | Post-Disinfection Chamber | 2.0 | 7.0-9.0 |
| | Conditioning | | |
| Phase 4 | System Clearing | 1.0 | 9.0-10.0 |

In some embodiments, the Phase 1 Chamber Conditioning phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5-1.0 minutes, between about 1.0-1.5 minutes, between about 1.5-2.0 minutes, between about 2.0-2.5 minutes, between about 2.5-3.0 minutes, between about 3.0-3.5 minutes, between about 3.5-4.0 minutes, between about 4.0-4.5 minutes, between about 4.5-5.0 minutes and any value in between those ranges listed, including endpoints. In some embodiments, the Phase 2 Disinfection Process phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5-1.0 minutes, between about 1.0-1.5 minutes, between about 1.5-2.0 minutes, between about 2.0-2.5 minutes, between about 2.5-3.0 minutes, between about 3.0-3.5 minutes, between about 3.5-4.0 minutes, between about 4.0-4.5 minutes, between about 4.5-5.0 minutes and any value in between those ranges listed, including endpoints. In some embodiments, the Phase 3 Post-Disinfection Chamber Conditioning phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5-1.0 minutes, between about 1.0-1.5 minutes, between about 1.5-2.0 minutes, between about 2.0-2.5 minutes, between about 2.5-3.0 minutes, between about 3.0-3.5 minutes, between about 3.5-4.0 minutes, between about 4.0-4.5 minutes, between about 4.5-5.0 minutes and any value in between those ranges listed, including endpoints. In some examples, the Phase 4 System Clearing can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5-1.0 minutes, between about 1.0-1.5 minutes, between about 1.5-2.0 minutes, between about 2.0-2.5 minutes, between about 2.5-3.0 minutes, between about 3.0-3.5 minutes, between about 3.5-4.0 minutes, between about 4.0-4.5 minutes, between about 4.5-5.0 minutes and any value in between those ranges listed, including endpoints.

In some embodiments, the method 3000 can start at step 3010. Prior to inserting the item(s) to be disinfected in the disinfection chamber 2050, the user must first clean and dry the items to be placed in the disinfection chamber 2050. Once the item is clean and dry, the user can place the disinfection chamber 2050.

The method 3000 can include step 3020—Phase 1 Chamber Conditioning—wherein the disinfection chamber 2050 is conditioned. In some embodiments, the chamber conditioning step of step 3020 can last for approximately 2.5 minutes. FIG. 26C provides an illustration of the method 3000 during the chamber conditioning.

As the name suggests, during the chamber conditioning of Phase 1, the ozone generator 2250 conditions the chamber for H₂O₂ disinfection by converting H₂O to OH radicals, thereby reducing residual moisture. In some embodiments, during the chamber conditioning phase of step 3020, ozone is supplied by the ozone generator 2250 to the disinfection chamber 2050 through a closed loop flow. Phase 1 Chamber Conditioning can optimize the disinfection chamber 2050 for disinfection. In some examples, the disinfection chamber 2050 is optimized for H₂O₂ disinfection.

A non-limiting example of the status of the components of the system 2000 during Phase 1 Chamber Conditioning of step 3020 is provided below:

| Element | Status |
|---|---|
| circulating fan 2100 | ON |
| opening 2410 of valve 2400 | OPEN (to internal circulation) |
| opening 2420 of valve 2400 | OPEN (to exhaust filter) |
| opening 2430 of valve 2400 | CLOSED (to inlet filter) |
| opening 2440 of valve 2400 | OPEN (to internal circulation) |
| opening 2510 of valve 2500 | OPEN (to internal circulation) |
| opening 2520 of valve 2500 | CLOSED |
| opening 2530 of valve 2500 | OPEN (to internal circulation) |
| pump 2600 (Pump 1) | ON (fills the nebulizer reservoir) |
| pump 2700 (Pump 2) | OFF |
| ozone generator 2250 | ON - first 1.5 min of 2.5 min phase |
| | OFF - last 1.0 min of 2.5 min phase |

As illustrated in FIG. 26C, the circulating fan 2100 is turned on to circulate air through the system 2000. The opening 2410 and opening 2440 of the valve 2400 and opening 2510 and opening 2530 of the valve 2500 can be opened to allow internal circulation of ozone. In some examples, the pump 2600 is “ON” which allows the nebulizer reservoir to fill. During Phase 1, the pump 2700 can be turned “OFF” as the chamber conditioning does not use any H₂O₂.

In some examples, the ozone generator 2250 is turned on for part of the chamber conditioning phase of step 3020. The ozone generator 2250 can be turned on for the first part of the chamber conditioning phase. For example, this can be the first 1.5 minutes of the 2.5 minute chamber conditioning phase. This can allow ozone to be supplied to the disinfection chamber 2050 from the ozone generator 2250 for a duration of time. As shown in FIG. 26C, opening 2410 and opening 2440 of the valve 2400 and opening 2510 and opening 2530 of the valve 2500 are opened to allow continuous circulation of ozone through the disinfection chamber 2050 in a closed loop flow. In particular, air flow occurs from the ozone generator 2250 to the disinfection chamber 2050 and from the disinfection chamber 2050 to the ozone generator 2250. The circulation of ozone during this phase conditions the chamber for H₂O₂ disinfection by converting H₂O molecules to OH radicals (disinfecting molecules) and thereby reducing residual moisture.

The ozone generator 2250 can be turned off for a second part of the chamber conditioning phase. In some embodiments, the ozone generator 2250 is turned off for the last 1.0 minute of the 2.5 minute chamber conditioning phase. During the second part of the chamber conditioning phase (i.e., the last 1.0 minute of the 2.5 minute phase), when the ozone generator 2250 is turned off, the ozone level will decay over time as it interacts with surfaces within the system 2000. In some embodiments, the sensor 2800 can achieve equilibrium with the outside pressure through the exhaust filter 2150. As shown, the opening 2420 of the valve 2400 can remain unsealed to ensure that no vacuum is created within the system 2000.

Method 3000 can include step 3030—Phase 2 Disinfection Process—wherein an item placed in the disinfection chamber 2050 is disinfected. In some examples, the Phase 2 Disinfection Process of step 3030 can last for approximately 4.5 minutes. FIG. 26D provides an illustration of the system 2000 during the Phase 2 Disinfection Process.

During the Phase 2 Disinfection Process, the disinfectant is introduced into the disinfection chamber 2050. In some embodiments, the disinfectant is a 50% hydrogen peroxide solution. The disinfectant can be introduced into the 2050 through the nebulizer 2300. The nebulizer 2300 can convert the disinfectant (i.e., the 50% hydrogen peroxide solution) from a liquid into a micro-spray that allows the disinfectant to move in the closed loop flow. In some embodiments, the micro-spray is the active ingredient used in the disinfection process.

A non-limiting example of the status of the components of the system 2000 during the Disinfection Process is provided below.

| Element | Status |
|---|---|
| circulating fan 2100 | ON |
| opening 2410 of valve 2400 | OPEN (to internal circulation) |
| opening 2420 of valve 2400 | CLOSED (to exhaust filter) |
| opening 2430 of valve 2400 | CLOSED (to inlet filter) |
| opening 2440 of valve 2400 | OPEN (to internal circulation) |
| opening 2510 of valve 2500 | CLOSED (to the chamber) |
| opening 2520 of valve 2500 | OPEN (to nebulizer and internal circulation) |
| opening 2530 of valve 2500 | OPEN (to nebulizer and internal circulation) |
| pump 2600 (Pump 1) | OFF |
| pump 2700 & nebulizer | ON (delivers 2.1 mL and nebulizes for 3.5 |
| 2300 (Pump 2 & Nebulizer) | min.) |
| ozone generator 2250 | OFF |

As illustrated in FIG. 26D, the circulating fan 2100 is turned on to circulate air through the system 2000. In some examples, the opening 2410 and opening 2440 of the valve 2400 are opened while the opening 2420 and opening 2430 are closed. In some embodiments, the opening 2520 and opening 2530 of the valve 2500 are opened while the opening 2510 is closed. The pump 2600 is turned “OFF” to prevent the disinfectant (e.g., 50% H₂O₂ solution) from filling the nebulizer 2300. In some examples, the pump 2700 and the nebulizer 2300 are turned “ON” to deliver disinfectant through the nebulizer 2300.

In some embodiments, 50% H₂O₂ is the active ingredient in the disinfection process of nebulizer 2300. During the disinfection process, the pump 2700 is a peristaltic pump that is fluidly connected to the nebulizer 2300. In some embodiments, the nebulizer 2300 is an 8-micron nebulizer mesh. The pump 2700 can be configured to deliver approximately 2.1 mL of 50% H₂O₂ disinfectant to the nebulizer 630 for the first 3.5 minutes of the contact time. In some embodiments, the system 600 can be designed to deliver a volume of 1.6 mL, 1.7 mL, 1.8 mL, 1.9 mL, 2.0 mL, 2.1 mL, 2.2 mL, 2.3 mL, 2.4 mL, 2.5 mL, between about 1.6 mL-1.7 mL, between about 1.7 mL-1.8 mL, between about 1.8 mL-1.9 mL, between about 1.9 mL-2.0 mL, between about 2.0 mL-2.1 mL, between about 2.1 mL-2.2 mL, between about 2.2 mL-2.3 mL, between about 2.3 mL-2.4 mL, between about 2.4 mL-2.5 mL and any value in between those ranges listed, including endpoints. In some embodiments, the pump 2700 is programed to deliver 0.01 mL of 50% H₂O₂ per second for 3.5 minutes (210 seconds) to the nebulizer 2300. The H₂O₂ solution can then be nebulized into a spray and be continuously circulated through the disinfection chamber 2050 in a closed flow loop. As shown in FIG. 26D, the openings of each of the valve 2400 and the valve 2500 are positioned to allow continuous circulation of disinfectant (i.e., 50% H₂O₂ disinfectant) through the disinfection chamber 2050 in a closed loop flow. In particular, air flow occurs from the nebulizer 2300 to the disinfection chamber 2050 and from the disinfection chamber 2050 disinfection chamber 2050, past the turned off ozone generator 2250, to the nebulizer 2300. In some examples, during the last minute of the disinfection processing phase, the nebulizer 2300 is turned off and the remaining H₂O₂ spray continues to circulate through the system 2000.

In some examples, during the disinfection process of step 3030, the residual ozone from Phase 1 of step 3020 decreases as H₂O₂ is introduced into the disinfection chamber 2050. Although it is known that ozone can be configured to neutralize H₂O₂, the volume of H₂O₂ introduced into the disinfection chamber 2050 during the disinfection process is sufficient to overcome those neutralizing effects. In some embodiments, the sensor 2800 can achieve equilibrium with the outside pressure through the exhaust filter 2150. As shown, the opening 2420 of the valve 2400 can remain unsealed to ensure that no vacuum is created within the system 2000.

The method 3000 can include step 3040—Phase 3 Post-Disinfection Chamber Conditioning—wherein the system 2000 clears the disinfection chamber 2050 of residual disinfectant. In some embodiments, the post-disinfection chamber conditioning of step 3040 can last for approximately 2.0 minutes. FIG. 26E illustrates system 2000 during the post-disinfection chamber conditioning.

During the post-disinfection chamber conditioning of Phase 3, ozone can be continuously supplied to the disinfection chamber 2050 through a closed loop flow. In some embodiments, the residual H₂O₂ micro-spray in the system 600 is neutralized.

A non-limiting example of the status of the components of the system 2000 during the Post-Disinfection Chamber Conditioning are provided below:

| Element | Status |
|---|---|
| circulating fan 2100 | ON |
| opening 2410 of valve 2400 | OPEN (to internal circulation) |
| opening 2420 of valve 2400 | CLOSED (to exhaust filter) |
| opening 2430 of valve 2400 | CLOSED (to inlet filter) |
| opening 2440 of valve 2400 | OPEN (to internal circulation) |
| opening 2510 of valve 2500 | CLOSED (to the chamber) |
| opening 2520 of valve 2500 | OPEN (to nebulizer and internal circulation) |
| opening 2530 of valve 2500 | OPEN (to nebulizer and internal circulation) |
| pump 2600 (Pump 1) | OFF |
| pump 2700 (Pump 2) | OFF |
| ozone generator 2250 | ON - 2 min |

As illustrated in FIG. 26E, the circulating fan 2100 is turned on to circulate air through the system 2000. In some examples, the valve 2400 is opened to internal circulation within the system 2000 but closed to the exhaust filter 2150 and the inlet filter 2200. In some embodiments, the valve 2500 is closed to the disinfection chamber 2050 but opened to the nebulizer 2300 and internal circulation within the system 2000. FIG. 26E illustrates that the circulation of air during post-disinfection chamber conditioning allows for ozone to circulate through the nebulizer 2300 and the disinfection chamber 2050. As noted above, this can allow for the ozone to neutralize any remaining H₂O₂. In some examples, the valve 2500 can be closed to the nebulizer 2300 but opened to the disinfection chamber 2050 within the system 2000.

During the Post-Disinfection Chamber Conditioning phase of 3040, ozone from the ozone generator 2250 is reintroduced into the disinfection chamber 2050 for 2 minutes and continuously circulated through the system 2000 in a closed loop flow. In some embodiments, as discussed previously, the residual H₂O₂ is neutralized by the ozone. After 2 minutes, the ozone generator 2250 is turned off. In some embodiments, the sensor 2800 can achieve equilibrium with the outside pressure through the exhaust filter 2150. As shown, the opening 2420 of the valve 2400 can remain unsealed to ensure that no vacuum is created within the system 2000.

The method 3000 can include step 3050—Phase 4 System Clearing—wherein fresh air is introduced into the system 2000 through the inlet filter 2200 to flush and purge the disinfection chamber 2050. The air can then exit the disinfection chamber 2050 and is exhausted through the exhaust filter 2150. In some embodiments, the inlet filter 2200 can be a HEPA filter. In some examples, the exhaust filter 2150 can include a HEPA filter and a carbon filter. In some embodiments the HEPA filters only allow things less than 0.3 μm particle size through the filter. The filtering of the inlet filter 2200 and the exhaust filter 2150 can ensure that only clean air leaves the system 2000 at the end of the method 3000. This final phase of the method 3000 can prepare the system 2000 for its subsequent use.

In some embodiments, the System Clearing phase of step 3050 can last for approximately 1.0 minute. FIG. 26F provides an illustration of the system 2000 during the Phase 4 System Clearing.

A non-limiting example of the status of the components of the system 2000 during System Clearing are provided below:

| Element | Status |
|---|---|
| circulating fan 2100 | ON |
| opening 2410 of valve 2400 | OPEN (to exhaust filter only) |
| opening 2420 of valve 2400 | OPEN (to exhaust filter) |
| opening 2430 of valve 2400 | OPEN (to internal circulation) |
| opening 2440 of valve 2400 | OPEN (to internal circulation) |
| opening 2510 of valve 2500 | CLOSED (to the chamber) |
| opening 2520 of valve 2500 | OPEN (to nebulizer and internal circulation) |
| opening 2530 of valve 2500 | OPEN (to nebulizer and internal circulation) |
| pump 2600 (Pump 1) | OFF |
| pump 2700 (Pump 2) | OFF |
| ozone generator 2250 | OFF |

As illustrated in FIG. 26F, the circulating fan 2100 is turned on to circulate air through the system 2000. In some examples, the openings of the valve 2400 are opened to allow air flow from the internal circulation out of the exhaust filter and for air flow into the internal circulation from the internal filter. The opening 2520 and opening 2530 of the valve 2500 are opened to allow airflow through the nebulizer chamber and internal circulation while opening 2510 is closed to the disinfection chamber 2050. As shown in FIG. 26F, the valve 2400 and valve 2500 provides for the circulation of air during system clearing to allow for fresh and filtered air to be pulled through the inlet filter 2200 and to circulate through the nebulizer 2300 and the disinfection chamber 2050. The air is then exhausted and filtered out of the exhaust filter 2150 to ensure that no ozone or H₂O₂ leaves the system. This system clearing phase ensures that the user is not exposed to harmful chemicals.

In some embodiments, the method includes an elevated temperature during the purge phase. In some embodiments, to elevate the temperature during the purge phase can include activating a heating element to increase the temperature of the walls. In some embodiments, the heating element can comprise ductile heating wires. In some embodiments, the ductile heating wires are embedded into the walls of the system. In some embodiments, the heating element can heat a carrier, like air, and a fan to circulate the air throughout the system. In some embodiments, the temperature conditioning increases the temperature of the walls sufficient to remove, for example, hydrogen peroxide.

### Implementation Mechanisms

According to some embodiments, the methods described herein can be implemented by one or more special-purpose computing devices. The special-purpose computing devices may be hard-wired to perform the techniques or may include digital electronic devices such as one or more application-specific integrated circuits (ASICs) or field programmable gate arrays (FPGAs) that are persistently programmed to perform the techniques or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, server computer systems, portable computer systems, handheld devices, networking devices or any other device or combination of devices that incorporate hard-wired and/or program logic to implement the techniques.

Computing device(s) are generally controlled and coordinated by operating system software, such as iOS, Android, Chrome OS, Windows XP, Windows Vista, Windows 7, Windows 8, Windows Server, Windows CE, UNIX, Linux, SunOS, Solaris, iOS, Blackberry OS, VxWorks, or other compatible operating systems. In other embodiments, the computing device may be controlled by a proprietary operating system. Conventional operating systems control and schedule computer processes for execution, perform memory management, provide file system, networking, I/O services, and provide a user interface functionality, such as a graphical user interface (“GUI”), among other things.

In some embodiments, the computer system includes a bus or other communication mechanism for communicating information, and a hardware processor, or multiple processors, coupled with the bus for processing information. Hardware processor(s) may be, for example, one or more general purpose microprocessors.

In some embodiments, the computer system may also include a main memory, such as a random-access memory (RAM), cache and/or other dynamic storage devices, coupled to a bus for storing information and instructions to be executed by a processor. Main memory also may be used for storing temporary variables or other intermediate information during execution of instructions to be executed by the processor. Such instructions, when stored in storage media accessible to the processor, render the computer system into a special-purpose machine that is customized to perform the operations specified in the instructions.

In some embodiments, the computer system further includes a read only memory (ROM), or other static storage device coupled to bus for storing static information and instructions for the processor. A storage device, such as a magnetic disk, optical disk, or USB thumb drive (Flash drive), etc., may be provided and coupled to the bus for storing information and instructions.

In some embodiments, the computer system may be coupled via a bus to a display, such as a cathode ray tube (CRT) or LCD display (or touch screen), for displaying information to a computer user. An input device, including alphanumeric and other keys, is coupled to the bus for communicating information and command selections to the processor. Another type of user input device is cursor control, such as a mouse, a trackball, or cursor direction keys for communicating direction information and command selections to the processor and for controlling cursor movement on display. This input device typically has two degrees F. of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

In some embodiments, the computing system may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression, or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules but may be represented in hardware or firmware. Generally, the modules described herein refer to logical modules that may be combined with other modules or divided into sub-modules despite their physical organization or storage

In some embodiments, a computer system may implement the methods described herein using customized hard-wired logic, one or more ASICs or FPGAs, firmware and/or program logic which in combination with the computer system causes or programs the computer system to be a special-purpose machine. According to one embodiment, the methods herein are performed by the computer system in response to hardware processor(s) executing one or more sequences of one or more instructions contained in main memory. Such instructions may be read into main memory from another storage medium, such as a storage device. Execution of the sequences of instructions contained in main memory causes processor(s) to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, or other types of storage devices. Volatile media includes dynamic memory, such as a main memory. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between nontransitory media. For example, transmission media includes coaxial cables, copper wire and fiber optics, including the wires that comprise a bus. Transmission media can also take the form of acoustic or light waves, such as those generated during radio-wave and infra-red data communications.

Various forms of media may be involved in carrying one or more sequences of one or more instructions to a processor for execution. For example, the instructions may initially be carried on a magnetic disk or solid-state drive of a remote computer. The remote computer can load the instructions into its dynamic memory and send the instructions over a telephone line using a modem or other network interface, such as a WAN or LAN interface. A modem local to a computer system can receive the data on the telephone line and use an infra-red transmitter to convert the data to an infra-red signal. An infra-red detector can receive the data carried in the infra-red signal and appropriate circuitry can place the data on a bus. The bus carries the data to the main memory, from which the processor retrieves and executes the instructions. The instructions received by the main memory may retrieve and execute the instructions. The instructions received by the main memory may optionally be stored on a storage device either before or after execution by the processor.

In some embodiments, the computer system may also include a communication interface coupled to a bus. The communication interface may provide a two-way data communication coupling to a network link that is connected to a local network. For example, a communication interface may be an integrated services digital network (ISDN) card, cable modem, satellite modem, or a modem to provide a data communication connection to a corresponding type of telephone line. As another example, a communication interface may be a local area network (LAN) card to provide a data communication connection to a compatible LAN (or WAN component to communicate with a WAN). Wireless links may also be implemented. In any such implementation, a communication interface sends and receives electrical, electromagnetic, or optical signals that carry digital data streams representing various types of information.

A network link may typically provide data communication through one or more networks to other data devices. For example, a network link may provide a connection through a local network to a host computer or to data equipment operated by an Internet Service Provider (ISP). The ISP in turn provides data communication services through the worldwide packet data communication network now commonly referred to as the “Internet.” The local network and Internet both use electrical, electromagnetic, or optical signals that carry digital data streams. The signals through the various networks and the signals on the network link and through a communication interface, which carry the digital data to and from the computer system, are example forms of transmission media.

In some embodiments, the computer system can send messages and receive data, including program code, through the network(s), the network link, and the communication interface. In the Internet example, a server might transmit a requested code for an application program through the Internet, ISP, local network, and communication interface.

The received code may be executed by a processor as it is received, and/or stored in a storage device, or other non-volatile storage for later execution.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or sub combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

1. A system for reducing the viability of microorganisms on a surface, comprising:
a housing comprising a front panel and a rear panel;
a filter holder positioned within the front panel, the filter holder comprising a first opening and a second opening for respectively receiving an inlet filter and an exhaust filter;
a chamber configured to receive an item to be disinfected, sterilized, or sanitized;
a cartridge engagement mechanism configured to receive a removable cartridge containing a volume of disinfectant/sterilant;
an ozone generation system comprising:
a duct comprising an inlet and an outlet, and
an ozone generator positioned along a length of the duct;
an evaporator comprising an inlet, an outlet, and a reservoir;
a nebulizer comprising: an inlet, an outlet, a chamber, and a cap, wherein the nebulizer, and wherein the nebulizer is fluidly connected to the reservoir of the evaporator;
a ducting comprising:
a blower,
a first pathway configured to receive an airflow from the blower,
a second pathway configured to deliver the airflow to the exhaust filter,
a third pathway configured to receive the airflow from the inlet filter, and
a fourth pathway configured to deliver the airflow to the ozone generation system;
a vent positioned on the rear panel of the housing, wherein airflow exits the housing through the vent; and
an ambient ozone sensor configured to detect a presence of ozone at a preset threshold value within the airflow, wherein airflow exiting the housing through the vent is configured to first flow past the ambient ozone sensor.

2. The system of claim 1, wherein the system is configured to stop operating if the ambient ozone sensor detects the presence of ozone above the preset threshold value.

3-10. (canceled)

11. The system of claim 1, wherein the front panel comprises a first door configured to seal the chamber.

12-16. (canceled)

17. The system of claim 1, wherein the inlet filter comprises an actuator that can be actuated if the inlet filter is not properly positioned or malfunctioning, wherein the exhaust filter comprises an actuator that can be actuated if the outlet filter is not properly positioned of malfunctioning, and wherein the system generates an error message to indicate to a user that at least one of the inlet filter and the exhaust filter is malfunctioning or improperly placed.

18. The system of claim 1, wherein the ozone generator further comprises a pair of bars comprising a pair of electrode centers extending through a pair of glass tubes, wherein each of the pair of electrode centers comprise aluminum.

19. (canceled)

20. The system of claim 1, wherein the ozone generation system further comprises a power supply, wherein the power supply is configured to provide a constant supply of voltage to the ozone generator.

21. (canceled)

22. (canceled)

23. The system of claim 1, wherein the duct of the ozone generation system comprises a plurality of fins that are configured to serve as guide vanes to direct airflow and to reduce pressure drops along a length of the duct of the ozone generation system.

24. The system of claim 1 wherein the ozone generation system further comprises an ozone sensor wherein the ozone sensor is configured to detect the amount of ozone in the airflow through the ozone generation system and is configured to adjust a duty cycle of the ozone generator to keep the concentration of ozone in the airflow within a preset range.

25. The system of claim 24, wherein the ozone sensor is configured to determine the density of air and to adjust a duty cycle of the ozone generator.

26-31. (canceled)

32. The system of claim 1, further comprising:
a reservoir pump configured to pump disinfectant/sterilant from the collection point of the reservoir to the chamber of the nebulizer; and
a cartridge pump configured to pump disinfectant/sterilant from the cartridge to the chamber.

33. The system of claim 1, wherein the cap comprises a curved surface that allows excess disinfectant/sterilant to flow back to the chamber of the nebulizer.

34. The system of claim 1, wherein the nebulizer further comprises a pizeocrystal, the piezocrystal configured to vibrate at a predetermined range to generate a mist of disinfectant/sterilant.

35-50. (canceled)

51. A system for reducing the viability of microorganisms on a surface, comprising:
a housing comprising a front panel and a rear panel;
an inlet filter;
an exhaust filter;
a chamber comprising:
a base,
an inlet panel on a first side of the chamber;
and outlet panel on a second side of the chamber;
a plurality of inlet openings on a first side of the base, and
a plurality of outlet openings on a second side of the base configured to receive an item to be disinfected, sterilized, or sterilized;
a cartridge containing a volume of disinfectant/sterilant;
an ozone generation system;
an evaporator comprising an inlet, an outlet, and a reservoir;
a nebulizer configured to convert hydrogen peroxide into a vapor, and wherein the nebulizer is fluidly connected to the reservoir of the evaporator;
a vent positioned on the rear panel of the housing, wherein airflow exits the housing through the vent; and
an ambient ozone sensor configured to detect a presence of ozone at a preset threshold value within the airflow, wherein airflow exiting the housing through the vent is configured to first flow past the ambient ozone sensor.

52-56. (canceled)

57. The system of claim 51, wherein the outlet panel further comprises a filter positioned behind the outlet panel and a filter support configured to retain the filter to the outlet panel.

58. A system for reducing the viability of microorganisms on a surface, comprising:
a housing comprising a front panel and a rear panel;
an inlet filter;
an exhaust filter;
a chamber configured to receive an item to be disinfected, sterilized, or sanitized;
a cartridge containing a volume of disinfectant/sterilant;
an ozone generation system comprising:
a duct comprising an inlet and an outlet, and
an ozone generator positioned along the length of the duct, the ozone generator comprising:
a pair of bars comprising a pair of electrode centers extending through a pair of glass tubes, wherein each of the pair of electrode centers comprise aluminum;
a power supply configured to provide a constant supply of voltage to the ozone generator,
an evaporator comprising an inlet, an outlet, and a reservoir;
a nebulizer configured to convert hydrogen peroxide into a vapor, and wherein the nebulizer is fluidly connected to the reservoir of the evaporator;
a blower,
a vent positioned on the rear panel of the housing, wherein airflow exits the housing through the vent; and
an ambient ozone sensor configured to detect a presence of ozone at a preset threshold value within the airflow, wherein airflow exiting the housing through the vent is configured to first flow past the ambient ozone sensor.

59. The system of claim 58, wherein the pair of bars extend parallel to the length of the duct of the ozone generation system.

60. (canceled)

61. (canceled)

62. The system of claim 58, wherein the duct of the ozone generation system comprises a plurality of fins that are configured to serve as guide vanes to direct airflow and to reduce pressure drops along a length of the duct of the ozone generation system.

63. The system of claim 58 wherein the ozone generation system further comprises an ozone sensor wherein the ozone sensor is configured to detect the amount of ozone in the airflow through the ozone generation system and is configured to adjust a duty cycle of the ozone generator to keep the concentration of ozone in the airflow within a preset range.

64. (canceled)

65. A system for reducing the viability of microorganisms on a surface, comprising:
a housing comprising a front panel and a rear panel;
an inlet filter;
an exhaust filter;
a chamber configured to receive an item to be disinfected, sterilized, or sanitized;
a cartridge containing a volume of disinfectant/sterilant;
an ozone generation system;
an evaporator comprising:
an inlet,
an outlet,
a reservoir comprising a collection point forming the lowest point of the reservoir and configured to retain excess disinfectant/sterilant,
a ducting comprising a central hub, a first pathway configured to deliver airflow from the inlet to the central hub, a second pathway configured to deliver airflow from the central hub, into the reservoir, and out of the outlet, a third pathway configured to deliver airflow from the central hub to the outlet, and
a valve positioned within the central hub, wherein the valve is configured to move between a first position and a second position;
a nebulizer configured to convert hydrogen peroxide into a vapor, and wherein the nebulizer is fluidly connected to the reservoir of the evaporator;
a blower;
a vent positioned on the rear panel of the housing, wherein airflow exits the housing through the vent; and
an ambient ozone sensor configured to detect a presence of ozone at a preset threshold value within the airflow, wherein airflow exiting the housing through the vent is configured to first flow past the ambient ozone sensor.

66. The system of claim 65, wherein the excess disinfectant/sterilant of the collection point is configured to flow into the nebulizer.

67-129. (canceled)
